(19) United States
(12) Patent Application Publication
(10) Pub. No.: US 2017/0011179 A1
(43) Pub. Date: Jan. 12, 2017

(54) Virtual Waiting Rooms In A Telemedicine System

(71) Applicant: MI Express Care Licensing Company, LLC, Canton, MI (US)

(72) Inventors: Jawad Ali Arshad, West Bloomfield, MI (US); Raheel Imtiaz, Lahore (PK); Waseem Ullah, Ann Arbor, MI (US); Marghub Alam Mirza, Gainesville, VA (US)

(73) Assignee: MI Express Care Licensing Company, LLC, Canton, MI (US)

(21) Appl. No.: 15/205,399

(22) Filed: Jul. 8, 2016

Related U.S. Application Data
(60) Provisional application No. 62/190,701, filed on Jul. 9, 2015.
(60) Provisional application No. 62/190,695, filed on Jul. 9, 2015.
(60) Provisional application No. 62/190,651, filed on Jul. 9, 2015.

(51) Int. Cl. G06F 19/00

(52) U.S. Cl. CPC G06F 19/327; G06F 19/3418; G06F 19/328; G06F 19/325; G06F 19/3406

(57) ABSTRACT

A telemedicine system including a care coordination software platform allows for patient monitoring at home and connects patients to their medical teams via telemedicine using a HIPAA compliant video portal augmented by remote assisted physical examination, performance of diagnostic testing including labs and x-rays, and provision of appropriate treatment and prescriptions. Medical care is provided at the patient's location without the patient having to travel or spend time in waiting rooms, provides treatment based on objective physical examination data and any appropriate diagnostic testing, and provides validation of patient identity. Healthcare providers are made available via online video encounters to communicate with patients. Allied healthcare workers are dispatched to be in physical proximity to the patient to assist in physical examination, and provide diagnostic data. Providers order appropriate treatments and prescriptions based on examination findings and diagnostics. The telemedicine system interfaces with medical sensors and collects data wired or wirelessly.

### REFERENCE TO PRIORITY APPLICATIONS

This application claims priority to U.S. Provisional Application No. 62/190,701, filed Jul. 9, 2015, entitled “Telemedicine And Mobile Health With Human Touch,” U.S. Provisional Application No. 62/190,695, filed Jul. 9, 2015, entitled “Fault Tolerant Identification Check Using Redundant Sensors And Information,” and U.S. Provisional Application No. 62/190,651, filed Jul. 9, 2015, entitled “Advance Radiology Package Containing Pictures Of A Body,” each of which is incorporated herein by reference in its entirety.

### FIELD OF THE DISCLOSURE

The subject matter disclosed herein relates to the field of telemedicine and more particularly relates to a system and method of providing virtual patient waiting rooms for use in a telemedicine system.

### BACKGROUND OF THE INVENTION

The conventional US healthcare system is considered by some as inefficient, inconvenient, and often fails to ensure good outcomes to treatment. Typical “fee-for-service” payment models give hospitals and physicians little financial incentive to reduce utilization of healthcare services, and often pay more for low-quality care that is ineffective than for higher-quality or other care that is more effective and can provide a good outcome to treatment. Recent Medicare reports indicate that as few as 5% of Medicare patients drive 40% of Medicare cost, and then next 15% of patients drive the next 40% of Medicare cost. The traditional face-to-face physician office visit is part of the problem due to several reasons.

One reason is poor care coordination. Patients who are discharged from the hospital or acute nursing facilities have significant confusion about their medications, as they are usually started on new medications, or have dose adjustments to their existing medications and are not provided with adequate information as to the use of these medications. This leads to lack in medication compliance and confusion further exacerbating the complexity of care of these patients. For example, patients taking cardiac medication to control blood pressure may not be informed of recommended changes to their lifestyle such as sitting before standing after laying for an extended period of time to equalize blood pressure and prevent a response which may require further hospitalization and increase costs.

A second reason is lack of timely health metrics. Intermittent office visits by patients which chronic medical conditions does not provide their physicians any visibility into the day-to-day status of their patients. Further, when patients cannot easily see their physicians, and physicians have no way of knowing the day-to-day condition of their patients, non-acute medical problems can quickly exacerbate, leading to an emergency visit and possible admission to the hospital.

A third reason is lack of access. It is often difficult for sick patients who need urgent services to coordinate schedules with their physicians, thus leading to increased utilization of emergency services.

For these and other reasons, global telemedicine (or telehealth) markets are projected to grow at a compound annual growth rate (CAGR) of slightly over 14% from $14 billion in 2014 to $35 billion by 2020. Recently, interest in telehealth has gained momentum due to its many benefits. For example, telehealth systems are especially useful for treating patients located in remote and inaccessible areas, such as many residents of Alaska, who would normally unable to obtain proper medical care in a reasonable amount of time. The benefits of telehealth systems, however, are not limited to remote areas as telehealth systems can also useful for patients with little spare time such as highly-stressed urban professionals who may skip necessary medical care due to workplace time restraints.

Unfortunately, conventional telehealth systems are modeled upon conventional healthcare systems and do not readily provide, to clinicians treating these patients such as physicians and the like, information that may be necessary for the proper treatment of these patients. For at least this reason, conventional telehealth systems are limited to certain types of medical fields. Further, conventional telehealth systems leave much to be desired due to, among other things, difficulty logging in, mistaken identity, delays, identity theft, service interruptions, and general user inconvenience. Although there have been attempts to overcome these and other disadvantages, these attempts have not been successful.

All current telemedicine solutions only allow healthcare providers to communicate with the patients without any human touch, and then base all medical decisions on subjective evaluation and history by the patient without any objective data from the patient or diagnostic tests.

Accordingly, there is a need for a telemedicine system that overcomes these and other disadvantages of conventional telemedicine systems.

### SUMMARY OF THE INVENTION

The present invention is a telemedicine system including a care coordination software platform that allows for patient monitoring at home and connects patients to their medical teams via telemedicine using a Health Insurance Portability and Accountability Act (HIPAA) compliant video portal that is augmented by remote and assisted physical examination, performance of any diagnostic testing including labs and x-rays, and provision of appropriate treatment and prescriptions.

Home monitoring allows medical teams to have visibility into patients' chronic diseases and allows intervention before these chronic diseases lead to complications. The telemedicine system provides easy access to healthcare providers for non-emergency conditions thus decreasing emergency room (ER) utilization and improving continuity of care. The system (1) provides medical care in a setting of patient's choice without requiring the patient to travel or spend time in waiting rooms; (2) provides treatment based on objective physical examination data and any appropriate diagnostic testing; and (3) provides validation of identity of the patient.

Patients with chronic diseases who are at high risk for complications are monitored using hardware that collects bio-data like blood pressure, blood sugar, pulse oximetry, weight, heart rate, etc. This data is analyzed by the software and notifications are generated for the medical team if and when the data points breach any parameters thus allowing the medical team to intervene before an addressable problem becomes an acute condition requiring hospitalization and/or ER visits. Healthcare providers are made available via online video encounters to communicate with patients. Allied healthcare workers are dispatched to be in physical proximity to the patient so they can assist in physical examination, and provide objective data including diagnostics. Providers provide appropriate treatments and prescriptions based on examination findings and diagnostics. A software app implementing the telemedicine system verifies identity of the patient by taking an image of driver's license using OCR technology and comparing it to the picture of the patient at the time of the encounter.

The telemedicine system can interface with proprietary and non-proprietary devices and collects data via wireless technology (e.g., Bluetooth, Wi-Fi, etc.) without manual input by the patient. The system then analyzes the data values automatically against pre-set parameters that are determined by the patient's provider, and are individualized and customizable. The system sends notifications to the care team if the values are outside the set parameters. Now the provider can have a telemedicine encounter with the patients, and send a healthcare worker to the patient's location to obtain more objective data and even assist the provider with an examination by using remote testing equipment. This allows the provider to treat the patient effectively leading to decreased complications.

Advantages of the telemedicine system of the present invention include: (1) monitoring patients at home without requiring data input from patient; (2) generating notifications for the healthcare team without requiring centralized monitoring stations with personnel thus improving cost structure of such monitoring; (3) connecting patient via video portal with the medical team; (4) verifying the identity of the patient; (5) allowing human interaction with the patient by having an allied health worker collect objective data like vital signs, assist the healthcare provider with an examination using equipment like stethoscope, otoscope, etc., and performing diagnostic testing like labs and x-rays; and (6) providing an accurate method of diagnosing medical conditions, and providing appropriate medical treatments including any prescriptions.

There is thus provided in accordance with the invention, a method of providing a plurality of virtual immediate waiting rooms, the method comprising providing on a server computer, a plurality of software queues, each queue associated with one of a plurality of different legal jurisdictions a patient may be located in, generating a graphical user interface that when rendered on a computing device displays at least an option for a patient to request an immediate encounter with a healthcare provider, determining a legal jurisdiction associated with the patent, placing the patient into a queue corresponding to the legal jurisdiction associated therewith, granting access to a queue to those healthcare providers licensed in the legal jurisdiction associated therewith, enabling a healthcare provider to select a patient from one or more queues they have access to and sending a notification thereof to the patient, and establishing an encounter between the healthcare provider and the selected patient.

There is also provided in accordance with the invention, a method of providing a plurality of virtual scheduled waiting rooms, the method comprising providing on a server computer, a plurality of software queues, each queue associated with one of a plurality of healthcare providers, receiving a request from a patient on a computing device to schedule an encounter with a health care provider, generating a graphical user interface that when rendered on a computing device displays an option for a patient to select from among a list of healthcare providers that meet one or more criteria, enabling a patient to select one of the healthcare providers on the list, sending a request to the server for an encounter between the patient and the selected healthcare provider at a desired date and time, creating an appointment for the patient with the selected healthcare provider at the desired data and time, placing the patient in the queue corresponding to the selected healthcare provider a predetermined time prior to the appointment, sending a notification to the patient when the selected healthcare provider is ready for the encounter, and establishing an encounter between the selected healthcare provider and the patient.

There is further provided in accordance with the invention, a method of providing a plurality of virtual scheduled waiting rooms for use on a computing device, the method comprising generating a graphical user interface that when rendered on the computing device displays an option for a patient to schedule an appointment for an encounter with a healthcare provider, receiving a request via the graphical user interface to schedule an encounter with a health care provider, generating a graphical user interface that when rendered on a computing device enables the patient to enter one or more criteria for selecting a healthcare provider, utilizing the criteria entered by the patient, querying a healthcare provider database located on a server computer accessible over the internet, wherein the server computer comprises a plurality of software queues, each queue associated with one of a plurality of healthcare providers, receiving in response from the server computer, a list of healthcare providers that satisfy the one or more criteria, generating a graphical user interface that when rendered on a computing device displays the list of healthcare providers, generating a graphical user interface that when rendered on a computing device enables the patient to select a healthcare provider on the list, sending a request to the server computer for an encounter between the patient and the selected healthcare provider at a desired date and time, and in response, the server computer creating an appointment for the patient with the selected healthcare provider at the desired data and time and placing the patient in the queue corresponding to the selected healthcare provider a predetermined time prior to the appointment, notifying the patient when the selected healthcare provider is ready for the encounter, and establishing an encounter between the selected healthcare provider and the patient.

### BRIEF DESCRIPTION OF THE DRAWINGS

The invention is herein described, by way of example only, with reference to the accompanying drawings, wherein:

FIG. 1 is a block diagram illustrating an example computer processing system or device adapted to implement the telemedicine system of the present invention;

FIG. 2 is a block diagram illustrating an example tablet/mobile computing device suitable for use with the telemedicine system of the present invention;

FIG. 3 is a diagram illustrating an example telemedicine system of the present invention;

FIG. 4 is a diagram illustrating an example encounter between a healthcare provider and a healthcare worker and patient at a remote patient location;

FIG. 5 is a diagram illustrating an example encounter between a radiological healthcare worker and a patient at a remote patient location;

FIG. 6 is a flow diagram illustrating an example identity validation method of the present invention;

FIGS. 7A, 7B, 7C, and 7D are a flow diagram illustrating an example healthcare provider workflow method of the present invention;

FIG. 8 is a diagram illustrating an example mobile device screenshot of a healthcare provider main landing screen;

FIGS. 9A, 9B, 9C, and 9D are a flow diagram illustrating an example healthcare worker workflow method of the present invention;

FIG. 10 is a diagram illustrating an example mobile device screenshot of a healthcare worker main landing screen;

FIGS. 11A, 11B, 11C, 11D, 11E, and 11F are a flow diagram illustrating an example patient workflow method of the present invention;

FIG. 12 is a diagram illustrating an example mobile device screenshot of a patient main landing screen;

FIG. 13 is a diagram illustrating an example immediate waiting room in more detail;

FIG. 14 is a diagram illustrating an example scheduled waiting room in more detail;

FIG. 15 is a diagram illustrating an example mobile device screenshot of patient appointment selection;

FIG. 16 is a diagram illustrating an example mobile device screenshot of patient healthcare provider selection;

FIG. 17 is a diagram illustrating a first example mobile device screenshot of estimated wait time for the encounter with the healthcare provider;

FIG. 18 is a diagram illustrating a first example mobile device screenshot indicating the selected healthcare provider type is not available;

FIG. 19 is a diagram illustrating a second example mobile device screenshot indicating the selected healthcare provider type is not available;

FIG. 20 is a diagram illustrating a second example mobile device screenshot of estimated wait time for the encounter with the healthcare provider;

FIG. 21 is a diagram illustrating a third example mobile device screenshot of estimated wait time for the encounter with the healthcare provider;

FIG. 22 is a diagram illustrating an example mobile device screenshot of a reminder for the encounter with the healthcare provider;

FIG. 23 is a flow diagram illustrating an example healthcare provider selection method;

FIG. 24 is a diagram illustrating an example mobile device screenshot of healthcare provider specialty type selection;

FIG. 25 is a diagram illustrating an example mobile device screenshot of requesting help in choosing a healthcare provider specialty type;

FIG. 26 is a flow diagram illustrating an example healthcare provider specialty type selection method;

FIG. 27A is a diagram illustrating a first example mobile device screenshot showing a human body for conveying location of current patient medical issue;

FIG. 27B is a diagram illustrating a second example mobile device screenshot showing a human body for conveying location of current patient medical issue;

FIG. 27C is a diagram illustrating a third example mobile device screenshot showing a human body for conveying location of current patient medical issue;

FIG. 27D is a diagram illustrating a fourth example mobile device screenshot showing a human body for conveying location of current patient medical issue;

FIG. 27E is a diagram illustrating a fifth example mobile device screenshot showing a human body for conveying location of current patient medical issue;

FIG. 27F is a diagram illustrating a sixth example mobile device screenshot showing a human body for conveying location of current patient medical issue;

FIG. 28A is a diagram illustrating a first example mobile device screenshot showing a human body with a list of possible medical issues based on the patient's selection;

FIG. 28B is a diagram illustrating a second example mobile device screenshot showing a human body with a list of possible medical issues based on the patient's selection;

FIG. 29 is a diagram illustrating an example mobile device screenshot showing recommended healthcare provider specialty types in accordance with the patient's selections;

FIG. 30 is a diagram illustrating an example mobile device screenshot of recommended healthcare provider specialty types in accordance with the patient's selections;

FIG. 31 is a diagram illustrating a screen shot of an example advanced radiology GUI generated in accordance with the present invention;

FIG. 32 is a flow diagram illustrating a method for performing an encounter in accordance with the present invention;

FIG. 33 is a diagram illustrating an example CTM invite message generated in accordance with the present invention;

FIG. 34 is a diagram illustrating an example GUI rendered on an HCP computing device in accordance with the present invention;

FIG. 35 is a diagram illustrating an example GUI rendered on a computing device of the patient in accordance with the present invention;

FIG. 36 is a diagram illustrating an example GUI rendered on the computing device of the HCW in accordance with the present invention; and

FIG. 37 is a diagram illustrating an example GUI rendered on a computing device of the CTM in accordance with the present invention.

### DETAILED DESCRIPTION

In the following detailed description, numerous specific details are set forth in order to provide a thorough understanding of the invention. It will be understood by those skilled in the art, however, that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary, for the understanding and appreciation of the underlying concepts of the present invention and in order not to obfuscate or distract from the teachings of the present invention.

Any reference in the specification to a method should be applied mutatis mutandis to a system capable of executing the method. Any reference in the specification to a system should be applied mutatis mutandis to a method that may be executed by the system.

### DEFINITIONS

The following definitions apply throughout this document.

The terms “telemedicine” and “telehealth” are both defined as the use of telecommunication and information technologies to provide clinical health care at a distance. Throughout this document the term telemedicine is used but is meant to incorporate telehealth as well. Telemedicine involves the distribution of health-related services and information. Distribution is via electronic information and telecommunication technologies. It allows long distance patient/clinician contact and care, advice, reminders, education, intervention, monitoring and remote admissions. As well as provider distance-learning; meetings, supervision, and presentations between practitioners; online information and health data management and healthcare system integration. For example, telemedicine can include two healthcare providers or workers discussing a case over video conference; a robotic surgery occurring through remote access; physical therapy done via digital monitoring instruments, live feed and application combinations; tests being forwarded between facilities for interpretation by a higher specialist; home monitoring through continuous sending of patient health data; client to practitioner online conference; or even videophone interpretation during a consult.

The term “telemedicine system” (or simply “system”) is defined as any system that provides and implements telemedicine or telehealth functionality.

The term “healthcare provider” (HCP) (or simply “provider”) is defined as any medical practitioner licensed to prescribe drugs and includes but is not limited to a medical doctor (MD), physician, doctor of osteopathic medicine (DO) advanced practice registered nurse, advanced practice provider (APP), podiatrist, veterinarian, etc.

The term “healthcare worker” (HCW) (or simply “worker”) is defined as any medical practitioner or medical support staff not licensed to prescribe drugs and includes but is not limited to a registered nurse (RN, BSN), licensed practical nurse (LPN), medic (EMT, MA), certified nursing assistant (CNA), radiology technician, proceduralist, pharmacy technician, phlebotomist, medic, psychologist, physician assistant, etc.

The term “patient” is defined as any person seeking healthcare services. The patient may be ill or injured and in need of treatment or medical assistance.

The term “care team member” (CTM) is defined as any friend, family, spouse, significant other, partner, etc. selected by the patient to help and aid in the care of the patient.

The term “sensor” is defined as any sensor or medicals sensor device such as a stethoscope, otoscope, mobile radiological scanning device, blood pressure monitor, blood oxygen sensors, tactile sensors, temperature sensors, pressure sensors, flow sensors, etc. that is capable of interfacing to a network or computing device through wired or wireless means.

The term “computing device” or “user station” (US) is defined as any general purpose device that has least one processing element, typically a central processing unit (CPU), and some form of memory and can be programmed to carry out a set of arithmetic or logical operations automatically. Examples of computing devices include but are not limited to smartphones (running Android, iOS, etc.), tablet computers (running Android, iOS, etc.), smartwatches (iWatch running watchOS, Samsung Gear, etc.), laptops and desktops (running macOS, Windows, UNIX, Linux, etc.). The computing device may be mobile or non-mobile.

The term “encounter” is defined as an interaction over a network between a healthcare provider and a patient. The interaction may be, for example, via video only, audio only, video and audio, text session, etc. or combination thereof.

Note that within the system, user access to various screens, functions, data and workflows are controlled by a set of roles and privileges associated with the particular user's user name. Role based control of access to protected health information (PHI) is a requirement for compliance with HIPAA regulations. Users may have more than one role in the system.

### Computer Embodiment

As will be appreciated by one skilled in the art, the present invention may be embodied as a system, method, computer program product or any combination thereof. Accordingly, the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present invention may take the form of a computer program product embodied in any tangible medium of expression having computer usable program code embodied in the medium.

The invention may be described in the general context of computer-executable instructions, such as program modules, being executed by a computer. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

Any combination of one or more computer usable or computer readable medium(s) may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or flash memory), an optical fiber, a portable compact disc read-only memory (CDROM), an optical storage device, a transmission media such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain or store the program for use by or in connection with the instruction execution system, apparatus, or device.

Computer program code for carrying out operations of the present invention may be written in any combination of one or more programming languages, including an object oriented programming language such as Java, Smalltalk, C++, C# or the like, conventional procedural programming languages, such as the “C” programming language, and functional programming languages such as Prolog and Lisp, machine code, assembler or any other suitable programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network using any type of network protocol, including for example a local area network (LAN) or a wide area network (WAN), or the connection may be made to an external computer (for example, through the Internet using an Internet Service Provider).

The present invention is described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented or supported by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowchart and/or block diagram block or blocks.

The invention is operational with numerous general purpose or special purpose computing system environments or configurations. Examples of well-known computing systems, environments, and/or configurations that may be suitable for use with the invention include, but are not limited to, personal computers, server computers, cloud computing, hand-held or laptop devices, multiprocessor systems, microprocessor, microcontroller or microcomputer based systems, set top boxes, programmable consumer electronics, ASIC or FPGA core, DSP core, network PCs, minicomputers, mainframe computers, distributed computing environments that include any of the above systems or devices, and the like.

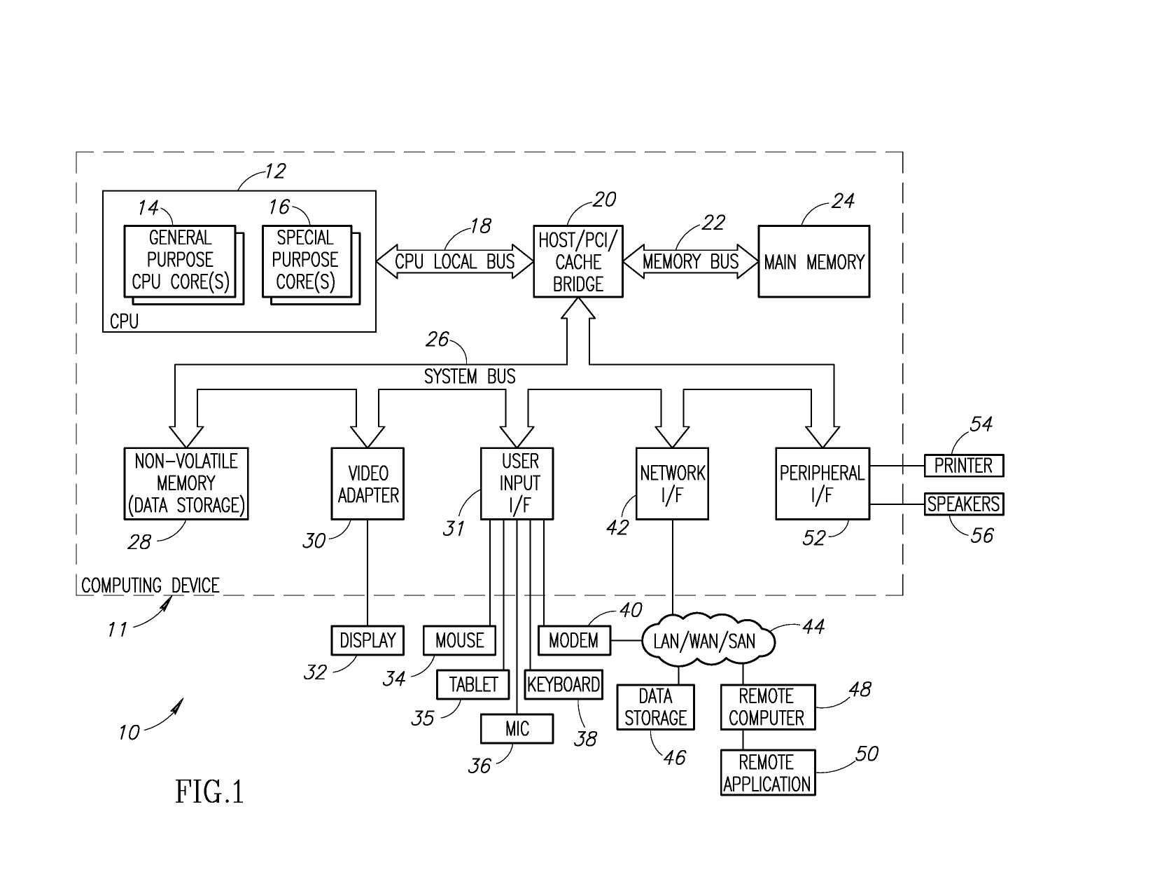

A block diagram illustrating an example computer processing system or device adapted to implement the telemedicine system of the present invention is shown in FIG. 1. The exemplary computer processing system, generally referenced 10, for implementing the invention comprises a general purpose computing device 11. Computing device 11 comprises central processing unit (CPU) 12, host/PIC/cache bridge 20 and main memory 24.

The CPU 12 comprises one or more general purpose CPU cores 14 and optionally one or more special purpose cores 16 (e.g., DSP core, floating point, etc.). The one or more general purpose cores execute general purpose opcodes while the special purpose cores execute functions specific to their purpose. The CPU 12 is coupled through the CPU local bus 18 to a host/PCI/cache bridge or chipset 20. A second level (i.e. L2) cache memory (not shown) may be coupled to a cache controller in the chipset. For some processors, the external cache may comprise an L1 or first level cache. The bridge or chipset 20 couples to main memory 24 via memory bus 20. The main memory comprises dynamic random access memory (DRAM) or extended data out (EDO) memory, or other types of memory such as ROM, static RAM, flash, and non-volatile static random access memory (NVSRAM), bubble memory, etc.

The computing device 11 also comprises various system components coupled to the CPU via system bus 26 (e.g., PCI). The host/PCI/cache bridge or chipset 20 interfaces to the system bus 26, such as peripheral component interconnect (PCI) bus. The system bus 26 may comprise any of several types of well-known bus structures using any of a variety of bus architectures. Example architectures include Industry Standard Architecture (ISA) bus, Micro Channel Architecture (MCA) bus, Enhanced ISA (EISA) bus, Video Electronics Standards Associate (VESA) local bus and Peripheral Component Interconnect (PCI) also known as Mezzanine bus.

Various components connected to the system bus include, but are not limited to, non-volatile memory (e.g., disk based data storage) 28, video/graphics adapter 30 connected to display 32, user input interface (I/F) controller 31 connected to one or more input devices such mouse 34, tablet 35, microphone 36, keyboard 38 and modem 40, network interface controller 42, peripheral interface controller 52 connected to one or more external peripherals such as printer 54 and speakers 56. The network interface controller 42 is coupled to one or more devices, such as data storage 46, remote computer 48 running one or more remote applications 50, via a network 44 which may comprise the Internet cloud, a local area network (LAN), wide area network (WAN), storage area network (SAN), etc. A small computer systems interface (SCSI) adapter (not shown) may also be coupled to the system bus. The SCSI adapter can couple to various SCSI devices such as a CD-ROM drive, tape drive, etc.

The non-volatile memory 28 may include various removable/non-removable, volatile/nonvolatile computer storage media, such as hard disk drives that reads from or writes to non-removable, nonvolatile magnetic media, a magnetic disk drive that reads from or writes to a removable, nonvolatile magnetic disk, an optical disk drive that reads from or writes to a removable, nonvolatile optical disk such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

A user may enter commands and information into the computer through input devices connected to the user input interface 31. Examples of input devices include a keyboard and pointing device, mouse, trackball or touch pad. Other input devices may include a microphone, joystick, game pad, satellite dish, scanner, etc.

The computer 11 may operate in a networked environment via connections to one or more remote computers, such as a remote computer 48. The remote computer may comprise a personal computer (PC), server, router, network PC, peer device or other common network node, and typically includes many or all of the elements described supra. Such networking environments are commonplace in offices, enterprise-wide computer networks, intranets and the Internet.

When used in a LAN networking environment, the computer 11 is connected to the LAN 44 via network interface 42. When used in a WAN networking environment, the computer 11 includes a modem 40 or other means for establishing communications over the WAN, such as the Internet. The modem 40, which may be internal or external, is connected to the system bus 26 via user input interface 31, or other appropriate mechanism.

The computing system environment, generally referenced 10, is an example of a suitable computing environment and is not intended to suggest any limitation as to the scope of use or functionality of the invention. Neither should the computing environment be interpreted as having any dependency or requirement relating to any one or combination of components illustrated in the exemplary operating environment.

In one embodiment, the software adapted to implement the system and methods of the present invention can also reside in the cloud. Cloud computing provides computation, software, data access and storage services that do not require end-user knowledge of the physical location and configuration of the system that delivers the services. Cloud computing encompasses any subscription-based or pay-per-use service and typically involves provisioning of dynamically scalable and often virtualized resources. Cloud computing providers deliver applications via the internet, which can be accessed from a web browser, while the business software and data are stored on servers at a remote location.

In another embodiment, software adapted to implement the system and methods of the present invention is adapted to reside on a computer readable medium. Computer readable media can be any available media that can be accessed by the computer and capable of storing for later reading by a computer a computer program implementing the method of this invention. Computer readable media includes both volatile and nonvolatile media, removable and non-removable media. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computer. Communication media typically embodies computer readable instructions, data structures, program modules or other data such as a magnetic disk within a disk drive unit. The software adapted to implement the system and methods of the present invention may also reside, in whole or in part, in the static or dynamic main memories or in firmware within the processor of the computer system (i.e. within microcontroller, microprocessor or microcomputer internal memory).

Other digital computer system configurations can also be employed to implement the system and methods of the present invention, and to the extent that a particular system configuration is capable of implementing the system and methods of this invention, it is equivalent to the representative digital computer system of FIG. 1 and within the spirit and scope of this invention.

Once they are programmed to perform particular functions pursuant to instructions from program software that implements the system and methods of this invention, such digital computer systems in effect become special purpose computers particular to the method of this invention. The techniques necessary for this are well-known to those skilled in the art of computer systems.

It is noted that computer programs implementing the system and methods of this invention will commonly be distributed to users on a distribution medium such as floppy disk, CDROM, DVD, flash memory, portable hard disk drive, etc. From there, they will often be copied to a hard disk or a similar intermediate storage medium. When the programs are to be run, they will be loaded either from their distribution medium or their intermediate storage medium into the execution memory of the computer, configuring the computer to act in accordance with the method of this invention. All these operations are well-known to those skilled in the art of computer systems.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or by combinations of special purpose hardware and computer instructions.

The methods described herein may be implemented on one or more processors. The method may store information generated and/or otherwise used by the method in memory for later use. The methods may include one or more steps which may be combined with other steps, separated into sub-steps, and/or skipped depending upon the particular implementation. The method may be performed by embodiments of the present system and may continue to execute after starting.

### Tablet/Mobile Computing Device

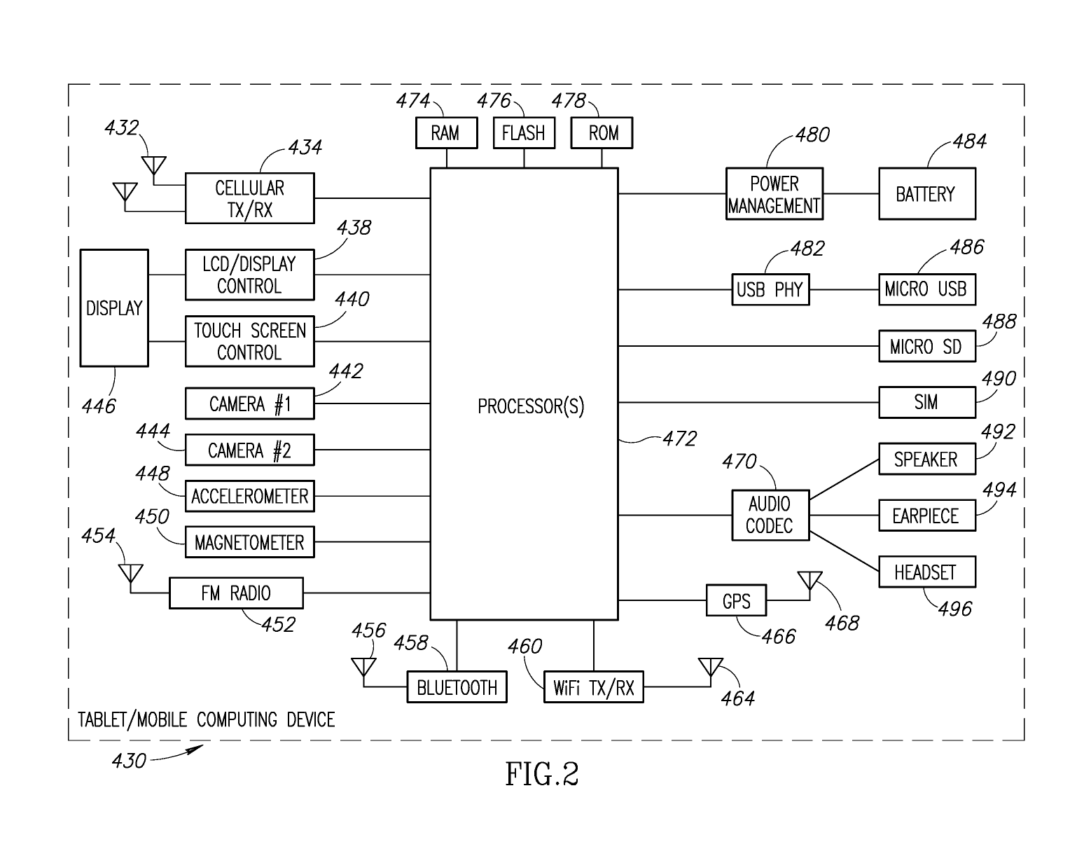

A high-level block diagram illustrating an example tablet/mobile computing device suitable for use with the telemedicine system of the present invention is shown in FIG. 2. The mobile computing device is preferably a two-way communication device having voice and/or data communication capabilities. In addition, the device optionally has the capability to communicate with other computer systems via the Internet. Note that the mobile device may comprise any suitable wired or wireless device such as multimedia player, mobile communication device, digital still or video camera, cellular phone, smartphone, PDA, PNA, Bluetooth device, tablet computing device such as the iPad, Surface, Nexus, etc. For illustration purposes only, the device is shown as a mobile device, such as a cellular based telephone, smartphone or superphone. Note that this example is not intended to limit the scope of the mechanism as the invention can be implemented in a wide variety of communication devices. It is further appreciated the mobile device shown is intentionally simplified to illustrate only certain components, as the mobile device may comprise other components and subsystems beyond those shown.

The mobile device, generally referenced 430, comprises one or more processors 472 which may comprise a baseband processor, CPU, microprocessor, DSP, etc., optionally having both analog and digital portions. The mobile device may comprise a plurality of cellular radios 434 and associated antennas 432. Radios for the basic cellular link and any number of other wireless standards and Radio Access Technologies (RATs) may be included. Examples include, but are not limited to, LTE 4G, Code Division Multiple Access (CDMA), Personal Communication Services (PCS), Global System for Mobile Communication (GSM)/GPRS/EDGE 3G; WCDMA; WiMAX for providing WiMAX wireless connectivity when within the range of a WiMAX wireless network; Bluetooth for providing Bluetooth wireless connectivity when within the range of a Bluetooth wireless network; WLAN for providing wireless connectivity when in a hot spot or within the range of an ad hoc, infrastructure or mesh based wireless LAN (WLAN) network; near field communications; UWB; GPS receiver for receiving GPS radio signals transmitted from one or more orbiting GPS satellites, FM transceiver provides the user the ability to listen to FM broadcasts as well as the ability to transmit audio over an unused FM station at low power, such as for playback over a car or home stereo system having an FM receiver, digital broadcast television, etc.

The mobile device may also comprise internal volatile storage 474 (e.g., RAM) and persistent storage 478 (e.g., ROM) and flash memory 476. Persistent storage also stores applications executable by the processor(s) including the related data files used by those applications to allow device 430 to perform its intended functions. Several optional user-interface devices include trackball/thumbwheel which may comprise a depressible thumbwheel/trackball that is used for navigation, selection of menu choices and confirmation of action, keypad/keyboard such as arranged in QWERTY fashion for entering alphanumeric data and a numeric keypad for entering dialing digits and for other controls and inputs (the keyboard may also contain symbol, function and command keys such as a phone send/end key, a menu key and an escape key), headset 496, earpiece 494 and/or speaker 492, microphone(s) and associated audio codec or other multimedia codecs, vibrator for alerting a user, one or more cameras and related circuitry 442, 444, display(s) 446 and associated display controller 438 and touchscreen control 440. Serial ports include a USB port (USB 1, 2, 3 or C) 486 and related USB PHY 482 and micro SD port 488. Other interface connections may include SPI, SDIO, PCI, USB, etc. for providing a serial link to a user's PC or other device. SIM/RUIM card 490 provides the interface to a user's SIM or RUIM card for storing user data such as address book entries, user identification, etc.

Portable power is provided by the battery 484 coupled to power management circuitry 480. External power is provided via USB power or an AC/DC adapter connected to the power management circuitry that is operative to manage the charging and discharging of the battery. In addition to a battery and AC/DC external power source, additional optional power sources each with its own power limitations, include: a speaker phone, DC/DC power source, and any bus powered power source (e.g., USB device in bus powered mode).

Operating system software executed by the processor 472 is preferably stored in persistent storage (i.e. ROM), or flash memory, but may be stored in other types of memory devices. In addition, system software, specific device applications, or parts thereof, may be temporarily loaded into volatile storage, such as random access memory (RAM). Communications signals received by the mobile device may also be stored in the RAM.

The processor, in addition to its operating system functions, enables execution of software applications on the computing device. A predetermined set of applications that control basic device operations, such as data and voice communications, may be installed during manufacture. Additional applications (or apps) may be downloaded from the Internet and installed in memory for execution on the processor. Alternatively, software may be downloaded via any other suitable protocol, such as SDIO, USB, network server, etc.

Other components of the mobile device include an accelerometer 448 for detecting motion and orientation of the device, magnetometer 450 for detecting the earth's magnetic field, FM radio 452 and antenna 454, Bluetooth radio 458 and antenna 456, Wi-Fi radio 460 including antenna 464 and GPS 466 and antenna 468.

In accordance with the invention, the mobile computing device is adapted to implement at least portions of the telemedicine system as hardware, software or as a combination of hardware and software. In one embodiment, implemented as a software task, the program code operative to implement the telemedicine system is executed as one or more tasks running on processor 62 and either (1) stored in one or more memories 474, 476, 478 or (2) stored in local memory within the processor 472 itself.

### Telemedicine System of the Present Invention

The telemedicine system provides for the diagnosis of a medical issue during an encounter with one or more HCPs. One or more users of the system may be located on-site (e.g., office) as well as off-site (e.g., patient's residence). A photo image of a patient wound is acquired by the patient and/or a CTM and uploaded to the system. An HCP views the photo image and can order a medical image, e.g., ultrasound, MRI, x-ray, of the wound and its surrounding areas. The system dispatches an HCW (or HCP) to the patient location to obtain the ordered information using a suitable mobile medical imaging device. The image of the wound is transmitted to an HCP located somewhere else and a diagnosis rendered remotely from the patient. The patient is then informed of the diagnosis during an encounter with the HCP.

Thus, the present invention provides diagnostics capability with enhanced information acquisition and distribution. This helps pinpoint an area of concern (e.g., a wound or other abnormality) using photo and medical images. Additionally, a HCW (e.g., medical imaging technician (MIT)) can be dispatched to the patient to obtain the medical images. A HCP can select a HCW and dispatch them to the patient's location to obtain vitals and medical images and perform tasks and orders. An HCW closest to the patient may be selected. An HCW closest to the patient, however, may be visiting another patient. In this case, the system selects an HCW that is not the closest but may visit the patient sooner. Thus, the system can employ learning to analyze schedules of HCWs such that it learns how long each HCW and/or groups of HCWs may take to perform their tasks during visits with patients. The system employs scheduling algorithms to select and dispatch an HCW that visits the patient in the shortest amount of time or a desired time as scheduled by the HCP or the patient.

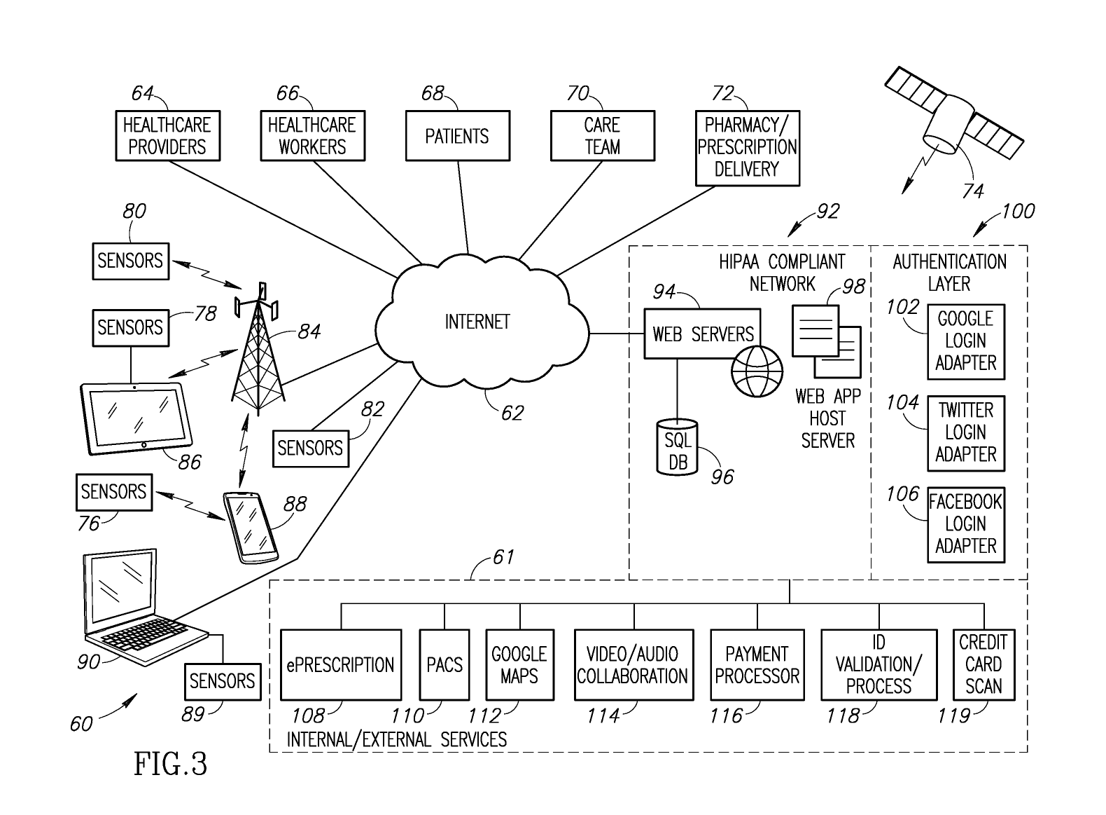

A diagram illustrating an example telemedicine system of the present invention is shown in FIG. 3. The system, generally referenced 60, comprises a wide area network such as the Internet 62, HIPAA compliant network 92, Global Positioning System (GPS) 74, healthcare providers 64, healthcare workers 66, patients 68, care teams 70, and pharmacy/prescription delivery 72. The system 60 also comprises mobile access such as via cellular or wireless system 84 for various mobile computing devices such as tablets 86, smartphones 88, and desktop/laptop computers 90. The system also comprises various network capable medical sensor devices such as stethoscopes, otoscopes, mobile radiological scanning devices, blood pressure monitors, etc. For example, sensors 82 communicate over the Internet 62, sensors 78 communicate via direct connection to tablet computing devices 86, sensors 76 communicate via wireless connection to smartphone computing devices 88, and sensors 89 communicate via either wired or wireless connection to a laptop, desktop, or web portal 90.

The HIPAA compliant network 92 comprises web services 94 coupled to the Internet and to one or more databases 96 (e.g., SQL, etc.), web app host server 98 and authentication layer 100 which comprises, for example, Google login adapter 102, Twitter login adapter 104, and Facebook login adapter 106. Connected to the network 92 are one or more internal or external services 61. Examples of services include but are not limited to ePrescription 108, Picture Archiving and Communication System (PACS) 110, Google Maps 112, video/audio collaboration 114, payment processing 116, ID validation/processing 118, and credit card scanning and processing 119.

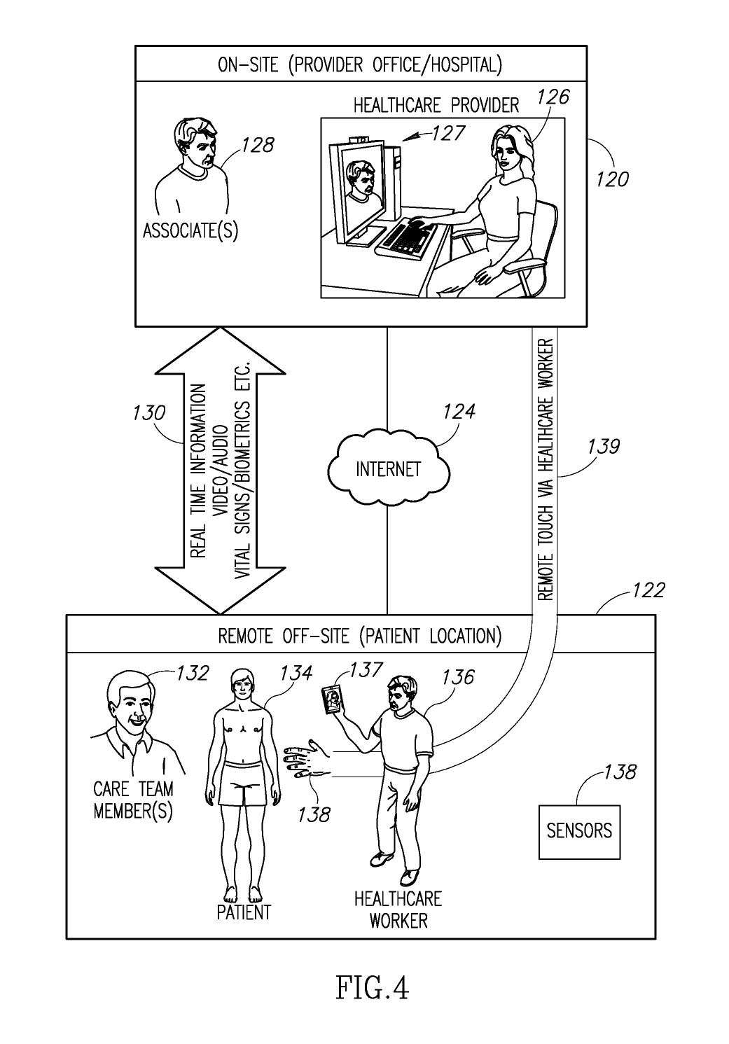

A diagram illustrating an example encounter between a healthcare provider and a healthcare worker and patient at a remote patient location is shown in FIG. 4. The system may include on-site 120 and off-site 122 locations which communicates with each other via a network 124 such as the Internet. The on-site location 120 can be situated at a non-mobile location of a provider such as in an office, a clinic, a hospital, and the like in which at least one US may be located for establishing communication between the US of a HCP 126 and/or associates 128 (e.g., HCPs, HCW, etc.) and a patient 134, one or more HCWs 136, and/or one or more CTMs 132 situated at the off-site location 122 (i.e. which may be remote from the on-site location) during an encounter (e.g., a video session). During the encounter, the system may acquire video information of parties to the encounter such as the HCP 126 and/or the associates 128 and the HCW 136, the patient 134, and/or the CTMs 132 and may transmit this video information to one or more other parties of the encounter and may thereafter render the transmitted video information in real time, as shown on the HCP computing device 127 and the HCW computing device 137. Accordingly, the system may establish at least one communication channel 130 to transmit the video information in real time.

In one embodiment, the at least one communication channel 130 may also transmit information related to the patient such as vitals, biometric information, etc. For example, one or more sensors 138 may acquire information such as vitals of the patient, medical images of the patient, etc., and may form corresponding sensor information which may be transmitted to the on-site portion 120 for further analysis and rendering on a device of the system for the convenience of the HCP 126. For example, a tactile glove sensor 138 is shown being used by the worker to examine the patient. Touch based feedback information detected by the glove is transmitted to the provider over link 139 where the provider can experience in real time what the worker feels at the patient location. Accordingly, the HCP 126 may effectively remotely touch the patient 134 via the HCW 136 and the one or more sensors 138.

Accordingly, the system may render information acquired by the sensors such as medical image information (e.g., from an MRI, X-ray, CT scan, and/or an ultrasound), blood test results, drug test results, acquired signals (e.g., ECGs, etc.), etc. on a rendering device of the HCP 126 for further analysis. The system may further provide an interface for the HCW 136 to enter analysis information regarding the patient 134 such as notes regarding the condition of the patient 134 such as the physiological and/or psychological condition of the patient 134 and may transmit this information to the HCP 126 for further analysis. The system may then transmit this information to the HCW 136 for further analysis as may be desired.

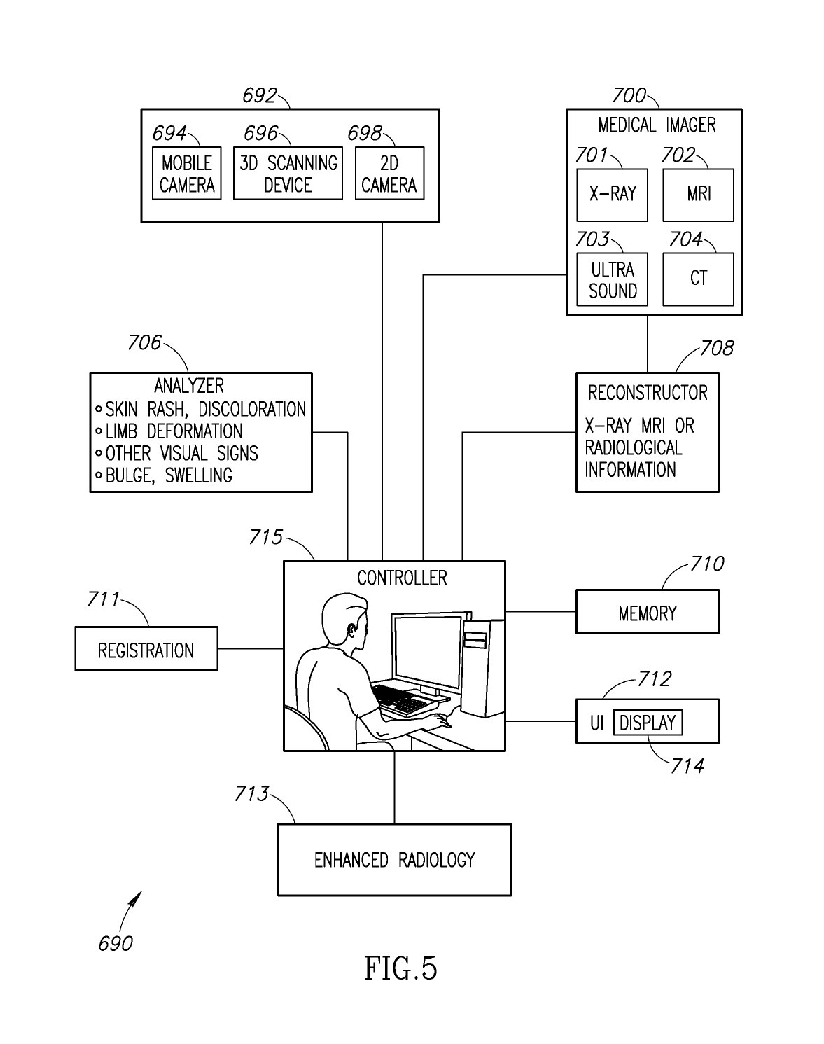

A computing system including a software application to integrate and/or combine image information obtained from one or more image acquisition devices such as cameras with radiology artifacts will now be described. A diagram illustrating an example encounter between a radiological healthcare worker and a patient at a remote patient location is shown in FIG. 5. The system, generally referenced 690, comprises part of an enhanced radiology platform of the telemedicine system constructed in accordance with the present invention. In one embodiment, the system comprises at least one controller 715, memory 710, USs 692, at least one medical imager 700, analyzer 706, reconstructor 708, registration block 711, user interface (UI) 712, and an enhanced radiology block 713.

In one embodiment, the controller 715 controls the overall operation of the system and may obtain US image information from an USs 692 of a patient and medical image information from the reconstructor 708.

The USs 692 may include a camera or other image capture device for capturing an image of the user. For example, the USs may comprise a smartphone, personal digital assistant (PDA), camera phone 694, a 2D camera 698 or 3D camera 696, and the like. The camera may capture still as well as video images. The USs may include a microphone to capture audio information concurrently with the image information. The images may have any desired definition (e.g., standard, high definition (HD), 4K, etc.) and may be black and white and/or color as may be acquired by the camera. The USs transmit the captured image information to the controller 715 for further analysis. One or more of the USs may belong to the patient and/or a CTM assigned to the patient. Accordingly, these images may be referred to as patient acquired images.

In one embodiment, the medical imager 700 includes one or more medical imagers and may be configured to acquire medical image information such as an X-ray imager 701, CT-scanner 704, MRI scanner 702, and ultrasound scanner 703. The medical imager may also comprise fluorescence-detecting cameras, catheter acquired images, and/or other medical imaging devices or medical scanning devices such as an electrocardiograph (ECG) or the like (not shown). The acquired medical image information can be provided in raw format or processed (e.g., reconstructed) format to the controller 715 for further processing such as for reconstruction, display, and/or storage. For the sake of clarity, these images may be referred to as medical images in the current example as opposed to the images captured by the USs 692.

In one embodiment, the reconstructor 708 is under control of the controller 715 and operative to obtain the medical image information acquired by the medical imager 700 (e.g., directly from the medical imager 700 or via the controller 715) and may reconstruct this information to form reconstructed medical image information and provide this information to the controller 715 for further processing. The reconstructed medical image information is rendered on the UI 712 of the system such as on a display 714.

In one embodiment, the memory 710 comprises any suitable memory such as a local and/or a distributed memory. The memory may store information generated or otherwise acquired by the system as well as operating instructions, user information, and/or other information such as firmware, etc.

In one embodiment, the UI 712 is under control of the controller 715 and includes any suitable UI which renders information such as enhanced radiology information, image information, reconstructed image information, and the like. The UI may include the display 714, a speaker, a haptic device, and/or a user input device such as a touchscreen display, a keyboard, a mouse, a microphone, a touchpad, a stylus, and/or any other device with which a user may enter information.

Once the medical image information, and/or the reconstructed medical image information is acquired, it can be attached to a radiology package linked to a patient and stored in association with patient information (PAI) for the corresponding patient. The patient radiology package may further include information related to an identification of an issue for which the patient sought treatment (e.g., right knee pain, etc.) and which issue and/or any information associated therewith may be date stamped. The system can generate a graphical user interface (GUI) with which the user may interact to store desired image information (e.g., from any source) in the patient radiology package.

In one embodiment, the analyzer 706 is under control of the controller 715 and is operative to obtain the image information acquired by one or more of the USs 692 and perform image analysis on the image information using any suitable method to detect any abnormalities such as a skin rash or discoloration, limb deformation, a bulge, swelling, other visual signs, etc. and form corresponding analysis information which can include information related to the abnormalities such as, a location of the abnormalities (e.g., in relation to an image and in relation to a patient), a description of the abnormalities (e.g., rash, bruise, cut, etc.), etc. Similarly, the analyzer 706 may analyze the reconstructed medical image information to detect abnormalities and mark them for later analysis. For example, when abnormalities are detected within an image (e.g., in the image information or the reconstructed medical image information), the analyzer 706 marks these abnormalities for further processing as may be desired. In another embodiment, the analyzer 706 uses highlighting or the like to delineate detected abnormal areas to mark these areas.

In another embodiment, the analyzer 706 determines or otherwise receives a description of the part of the patient that is being analyzed and/or marked (e.g., as abnormal) such as right arm, lower abdomen, left knee, etc. Accordingly, the analyzer 706 performs image analysis upon a corresponding image to determine the location of an abnormal area/region and mark this abnormal area/region and store this information in association with the corresponding image (e.g., image information or reconstructed medical image information). The analyzer may provide an interface (e.g., a GUI) with which the user can interface to mark any desired abnormal or other areas and store this information in association with the corresponding image (e.g., image information or reconstructed medical image information). The system can further include metadata which indicates whether the abnormal area/region was marked by a user and/or the system (e.g., automatically) and, if marked by a user include an identification of the user.

Thus, the analyzer 706 can mark any detected abnormal areas/regions within a region-of-interest (ROI) as may be determined by the analyzer 706 and/or a user. Accordingly, the system can render the image for a user to highlight the ROI or the system may determine a ROI within the image.

The analyzer 706 and/or user may further determine landmarks within the image information and/or medical image information and mark these landmarks for further analysis. These landmarks may then be stored in association with the corresponding image information and/or medical image information.

In one embodiment, the registration block 711 includes a software application and/or registers (i.e. links) one or more images obtained by one or more of the USs 692 (e.g., the image information) and/or one or more of the medical imagers 700 (e.g., the medical image information or reconstructed medical image information which may be collectively referred to as medical images for the sake of clarity) with each other and forms corresponding image registration information. For example, if a user (e.g., an HCP, a HCW, the patient, and/or a CTM) takes a picture of a patient's knee with an abnormality such as scaring on it (e.g., an image acquired by the USs 692), and an MRI image of this knee is available (e.g., a medical image), these images may be registered, when possible with each other. Similarly, images from the same or different imaging devices such as the USs 692 and the medical imagers 700 may be registered (i.e. linked) with each other provided that they are taken of the same or a substantially similar region-of-interest (ROI). For example, all images taken of a portion of an anatomy of a patient for an encounter (e.g., left knee pain, etc.) may be linked with each other. The registration portion 711 checks that the patient is the same patient and that the images were acquired substantially concurrently with each other such as within a certain time period (e.g., hours, a day, etc.) and/or for the same or similar ROI as may be set by system and/or user settings. The time period may be set by the system and/or user (e.g., three days, etc.).

The registration portion 711 can also obtain and/or detect landmarks within the image information and/or the medical image information which may aid in the linking process which may be performed automatically and/or semi-automatically (e.g., with at least some input from a user such as a provider or worker. The registration block 711 then forms image registration information that may be used to link the acquired image information.

In another embodiment, an initial encounter for an issue (e.g., left knee pain) may be considered a main encounter and may be assigned an identification that may be given a start date of the initial encounter for this issue (e.g., left knee pain Mar. 4, 2016) and all subsequent encounters for this issue may be associated with the main encounter identification. Thus, the system may separate multiple encounters for different issues so that images for these separate encounters may not have to be registered unless requested by a user such as a HCP. For example, if a patient has several encounters for left knee pain, and other encounters for a sore throat, images obtained for these separate issues may not have to be registered with each other. They may, however, be registered for the same issue.

After generating the image registration information and linking it with the corresponding images, registration block 711 may provide the image registration information to the controller 715 which stores this information in the corresponding patient radiology package in the memory of the system such as memory 710 for later use.

In one embodiment, the enhanced radiology block 713 is under control of the controller 715 and provides an interface with which a user may interact with the system to render information that was stored in the patient radiology package such as the image information (e.g., acquired by the cameras of the USs 692) and the reconstructed medical image information (e.g., acquired by the medical imagers 700) as well as related information such as image registration information, abnormalities, etc.

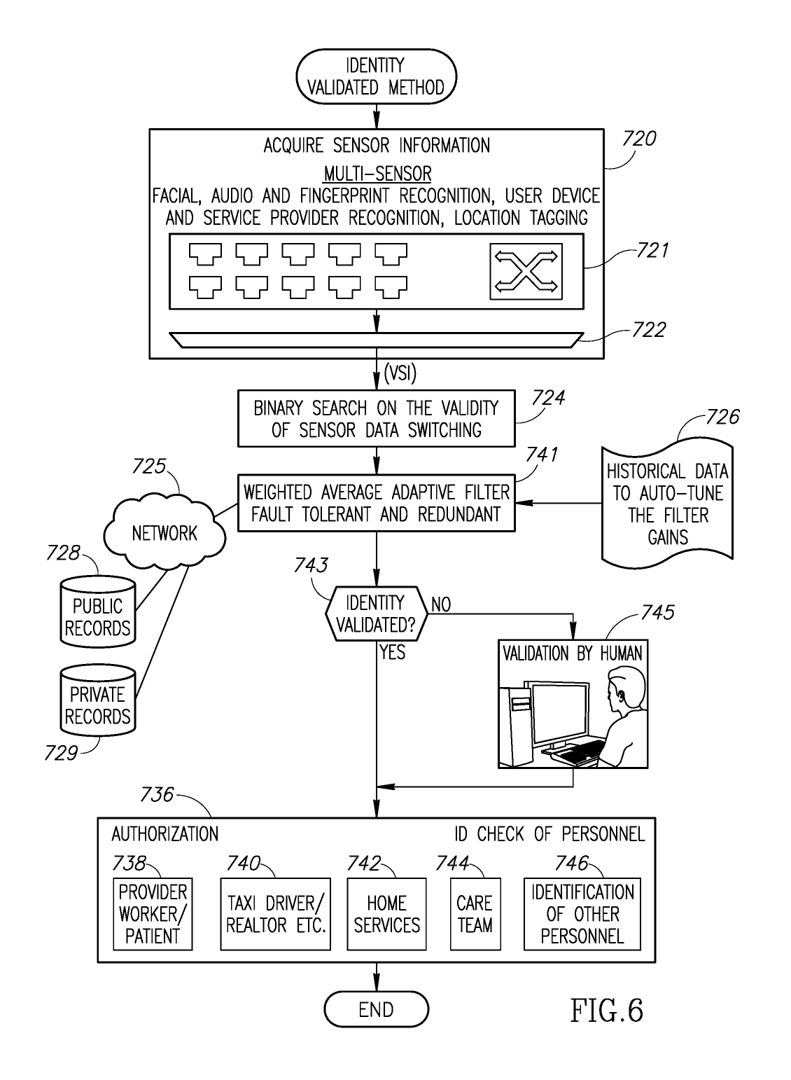

A flow diagram illustrating an example fault tolerant identity validation method of the present invention is shown in FIG. 6. In one embodiment, the method is implemented using one or more controllers, processors, shift registers, logic gates, computers, etc. of the system, etc. operating in accordance with the present system. The method may then store information generated and/or otherwise used by the method in a memory of the system for later use, if desired. The method may include one or more of the following steps or actions. Further, one or more steps of the method may be combined with other steps, separated into sub-steps, and/or skipped depending upon system settings.

The system first acquires information from one or more sensors 721 (step 720). The one or more sensors 721 generate corresponding sensor data which may be received by a multiplexer 722 which outputs corresponding validity sensor information (VSI) in any suitable format. The multiplexer 722 further obtains information from external sources such as from a service provider of the US and includes this information from the VSI as desired.

The sensors 721 can be divided into multiple types, namely biometric, virtual, and physical type sensors. For example, biometric type sensors acquire biometric information from a user and generate corresponding information such as image information (e.g., facial information such as a facial image obtained by a camera sensor of the system for use with facial recognition software and which is considered a facial image sample), audio information (e.g., a voice sample obtained by a microphone sensor of the system for use with voice recognition), fingerprint information (e.g., obtained by a fingerprint reader sensor of the system for use with fingerprint recognition and which is considered a fingerprint sample), which information may be stored in a corresponding format such as an image format (e.g., JPEG, etc.), audio format (e.g., MP3/MP4, etc.), and fingerprint format, respectively.

The virtual type sensors capture virtual information such as user station identification (USID) (e.g., a unique user device ID and/or service provider recognition information, cell phone ID (e.g., SSID), cell phone number, etc.), user name/password information, and/or location tagging information (e.g., obtained from a node at which a user is accessing the Internet, Internet service provider (ISP) ID, etc.). The physical type sensors obtain information such as location information (e.g., obtained from a location sensor such as a GPS sensor, etc.). At least one of the sensors is operative to determine the user/name password information and/or location tagging information and provide this information to multiplexer 722.

User name/password information is obtained via any suitable login such as a user manually logging in to the system using, for example, a text entry area to enter the user's ID and corresponding password. Accordingly, the system can generate a GUI including a request to enter a user ID (e.g., a name, an email address, a telephone, number, etc.) and password and renders the GUI on the US for input by the user.

With regard to acquisition of the sensor information, the sensor information may be acquired from a user actively or passively. For example, with respect to passive acquisition of the sensor information, it is envisioned that a user may enter his/her fingerprint when requested or the fingerprint of the user may be obtained when the user touches the screen such as during a screen unlock process in which the user touches the screen to unlock the screen. Fingerprint data can also be acquired when a user selects a selection item (e.g., a menu item) which is rendered on a touchscreen display of the US. The menu item may be related to any GUI rendered by the US. Similarly, the system can detect the face of a user when the user uses his/her US and generates corresponding facial (image) information. The sensor information may include information which identifies the type of sensor information (e.g., fingerprint, facial recognition, audio, etc.).

Conversely, with regard to active acquisition of validity sensor information (VSI), the system can generate and render a request for the user to enter information for the VSI such as an image of the user's face, a fingerprint, etc. Accordingly, the user enters the desired information (or portions thereof) by selecting a menu item to activate a camera of the US to capture a facial image of the user, or by placing a finger over a fingerprint reader of the system, etc.

Note that some the sensor information may be required. For example, sensor information which may identify an account of a user (e.g., user ID, name, biometric information, etc.). Further, for accounts not yet established, sensor information may require a name to be entered. Other identifying information may be obtained (as may be set by system settings, etc.) such as date of birth, social security number, etc.) which may be used to validate a user and/or to initialize an account of the user.

The system can also provide the user with options to select which sensor information to acquire. For example, a user may select to enter facial recognition, fingerprint, and location information, while a different user may select to enter facial, voice, and location information. These settings can be set in user setting information (USI) for later use by the system. Accordingly, at login, the system obtains USI for the user (e.g., via identifying US of the user). The USI can be analyzed to select sensor information to acquire for the user. Thus, it may be desirable to select different types of sensors to use to acquire the sensor information. For example, when at a home registered to a user, location tagging information is entered (which the system recognizes as belonging to a registered location of the user) for at least part of the sensor information. When the user is travelling, however, a voice sample for at least part of the sensor information is preferred rather than the location tagging information which will not be recognized as belonging to an account of the user. Further, depending upon the type of sensor used, the system can generate a query to obtain the sensor information. For example, if an ISP ID of the US of a user is desired, the system generates a query to request this information and submits this information to the ISP or accesses this information from the memory of the US.

Table 1 below shows login information selection data which includes login information selections for two registered users (e.g., user A and user B) of the system. Each user has corresponding login information selections.

TABLE 1
Login Information Selection Data

| Sensor Information | | |
|---|---|---|
| (Minimum No. Samples = 3_) | User A | User B |
| Account Name | Yes | Yes |
| Audio Sample | Yes | Yes |
| Facial Image Sample | Yes | Yes |
| Fingerprint Sample | Yes | No |
| Iris Sample | No | Yes |
| Location Tagging | No | No |
| . | . | . |
| . | . | . |
| . | . | . |
| Service Provider | No | No |

As indicated in Table 1, User A desires to use sensor information for login such as an account name, audio sample, facial image sample, and fingerprint sample for logging in, whereas user B, wants to use an account name, audio sample, facial image sample, and iris sample. The system sets a minimum number of samples required for the login between one and five, e.g., three. In addition, one or more of the selections and/or groups of selections may be mandatory. For example, is may be required to enter a user name to reduce processing. A user, however, may enter a fingerprint and the system may identify the user based upon the fingerprint by searching a fingerprint library and finding a match.

Each user can set and reset information within the login information matrix. For example, a user may set or reset one or more selections within the login information matrix. The login information matrix, however, may be blocked from being set/reset by a user. Further, the system may employ a default logon information matrix as may be desired.

Sensor information may be stored in the multiplexer (mux) 722 prior to output. The mux then outputs the sensor information as the VSI to the binary searcher 724. in one embodiment, location tagging is achieved by determining a location of the us and generating corresponding location information which is then included within the VSI with, for example, the image, audio, or finger print information, etc., as desired.

The system then performs a binary search on the validity of the sensor data included within the VSI (step 724). The binary searcher receives the VSI and identifies the sensor information contained therein using any suitable method. For example, for biometric information such as an image, voice, and fingerprint information, this information may be extracted from the VSI and identified (e.g., by type) using any suitable method such as by identifying a format of the information. For example, the binary searcher can identify image information as a facial image sample, audio information as a voice sample, and fingerprint information as a fingerprint sample of the user.

The binary searcher then obtains validation information to validate at least a portion of the sensor information extracted from the VSI. This validation information can be obtained from user account information stored in association with an account of the user and/or the system can identify a user based upon a match of the sensor information against stored validation information (e.g., a match of a name of the sensor information with a corresponding name stored in the user account information, a match of a fingerprint sample of the sensor information with a fingerprint sample stored in the user account information, etc.). Once at least some of the sensor information is matched with corresponding stored validation information of a user, a partial identification of a user may be established and the system then attempts to match the remaining sensor information of this user with corresponding validation information for this user.

Thus, for example, if the system obtains a fingerprint sample from the user, the system matches this fingerprint sample to a fingerprint sample within the verification information and at least partially identifies the user. Thereafter, the system, depending upon system settings, obtains verification information for this user from the memory of the system and attempts to match the remaining sensor information with corresponding information within the verification information. Thus, the system obtains verification information for a particular user or identifies a user based upon verification information for a plurality of users and, if a match is found (e.g., the user is at least partially identified), the system obtains further verification information for this user to more fully identify them.

The system compares the sensor information within the VSI to determine whether it matches corresponding verification information stored in the memory of the system. For example, assuming an image (facial) and fingerprint information of the user are acquired (and included within the VSI), the system compares this information with corresponding image and fingerprint information for the user that is stored in VSI memory and determines whether there is a match. The sensor information as well as the verification information can be passively or actively acquired. For example, the system can passively obtain facial image information as sensor information and then employs facial recognition methods to determine whether the acquired facial image information matches the corresponding facial image information for the user. The system then stores results of the determination(s). For example, assuming that the user name, facial image information, and fingerprint sample were matched with corresponding verification information, the system stores the results. If, however, the user name matched the verification information but the facial image information and fingerprint sample did not match corresponding verification information for the user name, the system also stores results of this determination. The user can then be given another opportunity to get their ID validated.

Thus, the binary searcher determines whether the sensor information matches corresponding validity information. If the sensor information matches the corresponding validity information, the sensor information and/or results of the determination are input to the weighted average adaptive filter (step 741). Otherwise, the user is not identified and is given another opportunity to get validated. For example, the user may have incorrectly entered a user name, etc.

Note that the system may identify a user even when all the sensor information is not input and/or not matched with corresponding validity information. For example, if it is determined that some sensor information which matches the corresponding validity information is greater than a threshold value such as N_{Thresh} (where N_{Thresh} is an integer set by the system and/or a user), the process determines that the sensor information matches the corresponding validity information.

In the event that the user is not identified, which, may occur when the user enters an incorrect user name, or does not enter a fingerprint sample correctly, or is not registered, the system informs the user (e.g., notifying the user that a fingerprint match was not made, user ID is incorrect, etc.). The user is given an opportunity to correct the sensor information. For example, if the sensor information for the user does not match any verification information for any accounts of a user, the system may determine that the user is not a registered user, and may inform (e.g., by displaying a message) the user that the user has not been recognized as a registered user and may provide the user with an opportunity to correct and/or reenter the sensor information. Accordingly, the system repeats step 720.

The weighed adaptive filter (step 741) obtains the sensor information from the binary searcher and results of the determinations for the identified user. The system then pulls validity search information (VSI) from any suitable information source such as from public 728 and/or private 729 records databases for the identified user via network 725 (such as the Internet). For example, the VSI may include information such as licenses (e.g., drivers and other licenses), a passport, banking account(s), criminal records, and service provider(s) of/for the identified user and/or the US the user is communicating with. The system forms the VSI such that the VSI includes information fields which correspond with information fields of the sensor information. For example, if the sensor information includes a facial image information field, the system includes a corresponding facial image information field and generates a corresponding query to obtain the VSI from any suitable source such as public and/or private records databases.

The system determines whether fields of the VSI match corresponding fields of the sensor information and generates corresponding matching information (MI) which indicates whether a match has been found. The MI can be a binary number whether or not the sensor information matches the VSI (e.g., “1” or yes, or “0” or no) and/or may be normalized to a likelihood of a fit (e.g., 0 to 1, where “0” indicates no likelihood of a fit (i.e. no match) and “1” indicates a full fit (i.e. a match). For clarity sake, it is assumed that the MI is a binary number (e.g., “1”=match and “0”=no match). Accordingly, the system employs well known statistical algorithms which analyze the sensor information and VSI to determine a likelihood of a match (e.g., a fit) and generate the corresponding MI.

In one embodiment, the system applies a weight to the MI. For example, the result of a name match (e.g., name MI) has a weight of 10*MI, while the result of a location may have a weight of one, wherein higher weights may indicate greater importance and vice versa. Thus, a weight of one can be assigned to matches of lowest importance and a weight of 10 may be assigned to those of greatest importance. The system may determine weights from a weight table which include weights for various information fields such as a weight for a name field (e.g., 10) and a weight for an address field (e.g., one), etc. The system can obtain the weight table from memory and determine corresponding weights for matches in accordance with the weight table.

In one embodiment, the databases and/or queries are based upon a group and/or subgroup that the identified user is a member of, such as, a patient, HCP, HCW, CTM, and/or subgroup thereof. The system further includes an unregistered users group which may include unregistered users who may be attempting to register as a member of another group of the system (e.g., an HCP, patient group, etc.). For example, if the identified user is identified as a provider, the system may determine that the identified user is a member of the HCP group and physician subgroup. The system then obtains information related to this group and subgroup (e.g., searching terms) and further obtains locations (e.g., databases, websites, etc.) from which to search. The system obtains search terms using any suitable method such as by employing a table look up method to obtain desired search terms. For example, assuming the identified user is a provider (e.g., of the HCP group and physician subgroup), the system obtains search information for the group and subgroup from a search information table (SIT) stored in memory.

Table 2 below shows a portion of a SIT for a HCP group and its subgroups (e.g., physicians, PAs, and NPs). Other groups may have similar SITs with information fields which can vary by group and/or subgroup. The SIT can be learned and/or modified by the system or modified by a user.

TABLE 2
Search Information Table (SIT)

| Group | Search Information Fields | | | |
|---|---|---|---|---|
| HCP | (for validation search | Check | Databases and Location | |
| Subgroup | information (VSI)) | Fields | Public | Private |
| Physician | name, address, profession, | registration | State: | Insurance Co. |
| | license no. date of | end date | CA: If MD: | A: |
| | licensure, additional | dated after | xww.mbc.ca.gov/Breeze/License_Verification.aspx | xyz.123 |
| | qualification, status, | current date | CA: If DO: | Insurance Co. |
| | registration end date, | | xww.breeze.ca.gov/datamart/searchByName.do | B- . . . |
| | medical school, and | | . . . . . . | |
| | degree date | | NY: xww.nys.op . . . | |
| | | | NJ: xww.nj . . . | |
| | | | . . . | |
| PA | name, address, profession, | registration | www.xyz123.com | Ins. Co. ABC |
| | license no. date of | end date | | |
| | licensure, additional | dated after | | |
| | qualification, status, | current date | | |
| | registration end date, | | | |
| | medical school, and | | | |
| | degree date | | | |
| NP | name, address, profession, | registration | www.xyz123.com | Ins. Co. ABC |
| | license no. date of | end date | | |
| | licensure, additional | dated after | | |
| | qualification, status, | current date | | |
| | registration end date, | | | |
| | medical school, and | | | |
| | degree date | | | |

As shown in Table 2, the system identifies a group/subgroup of the identified user (e.g., a physician subgroup of the HCP group). The system then generates a query in accordance with the search information field for the current group and subgroup (e.g., in the current example, the query is formed to obtain: name, address, profession, license no., date of licensure, additional qualification, status, registration end date, medical school, and degree date). The search query is submitted to the corresponding information source(s) (e.g., databases) for the current group and subgroup as listed in Table 2.

The sources (e.g., databases) to search may be determined in accordance with a geographical region of the user (e.g., Michigan, etc.). For example, assuming that the identified user has a residence or business address in Michigan, the system performs a search in databases corresponding with this state such as the Michigan Licensing and Regulatory Affairs website and/or in neighboring states such as in Ohio, Indiana, Wisconsin, etc. This may save system resources. Accordingly, each state, territory, and/or region may have corresponding search locations associated therewith and is listed in the SIT.

Thus, in one embodiment, the search includes terms which correspond with the search information fields of the SIT and can be further narrowed to search databases in accordance with an identified geographic region associated with the user. Assuming that the user is identified as being licensed to practice their profession in the state of Michigan, then information from this state may be obtained. One or more private databases (such as insurance company databases, private databases, etc.) are queried for the same or similar information or information that cannot be obtained (or is difficult to be obtained) from public sources (e.g., public database) or vice versa. Accordingly, the system transmits a search query to request the information from a desired source (e.g., a private and/or public database) which corresponds with the search information fields. The system receives a response to the query and generates corresponding the VSI.

Note that sources may include a list of search addresses for searching each group and/or subgroup. Further, the search addresses may be geographically dependent upon geographic location. Thus, for example, if the user is licensed in the Michigan, the search may query for information related to that state such as the Licensing and Regulatory Affairs website, Michigan Department of Motor Vehicles website (e.g., for license information, etc.), etc., public utility service providers (e.g., for phone and/or Internet service related records such as for address) and banks (e.g., for banking related records such as address) operating within the identified users geographic region (e.g., Michigan area). This can save time and system resources. A search can be widened to cover other geographic areas such as when an insufficient number of records (e.g., a number of records below a record threshold) are obtained through a narrowed geographic region search and/or when information falls below a quality or reliability threshold.

The system can obtain driver license information (e.g., including name, address, license number, and biometric information which may include a facial image and/or a fingerprint(s) of the licensee) or non-driver ID for the identified user from any suitable identification source (e.g., database) such as an insurance company database, a state motor vehicle department database, a health insurance provider, etc. Similarly, passport information can be obtained for the user from an immigration database (e.g., provided by a state or federal immigration department) which may include similar information as that discussed above with respect to a driver license. Lastly, banking information related to bank accounts, credit cards, mortgages, and rental property can be obtained from any suitable banking database.

Assuming that the sensor information includes a user name, facial image information and fingerprint sample, the system can then query any suitable source to obtain at least corresponding information such as driver license information (e.g., a driver license database, an insurance database, etc.) and generate corresponding VSI. The information included in the sensor information, such as the user name, facial image information and fingerprint sample is compared with the corresponding information (e.g., user name, facial image information and fingerprint sample, respectively) from the driver license information for the identified user in order to determine whether a match (or substantial match) is found as well as to generate corresponding MI which indicates whether the compared information matches. In other words, the matching information (MI) is generated to indicate a degree of confidence (e.g., probability of match) in the match. The MI may be normalized as well.

In one embodiment, the system obtains similar information fields and compares them to determine a match (or a substantial match) and generates the corresponding MI. For example, the system queries several databases for an address of the identified user. The system then compares results of these comparisons (e.g., residence address, professional address, insurance carrier, social security number, etc.) and determines whether a match (or a substantial match) is found. The MI includes information related to results of a comparison as well as the compared information, the sources, and corresponding time stamps (e.g., indicating time of the search query). Thus, for example, if several databases are queried for an address of the identified user and these addresses are determined not to match, the MI includes results of the comparison, a flag to indicate fields which did not match, sources and of the information that was compared (e.g., address 1 obtained from insurance sources A and address 2 obtained from insurance source B, on Jan. 1, 2017).

In one embodiment, the MI is generated using any suitable format and stored in memory and associated with the identified user for later use. the MI includes a format identifier, e.g., a certain number of bits may indicate the information contained therein.

The VSI is then searched for terms which are not permitted for a user depending upon the group and subgroup of the identified user. For example, with reference to Table 2, the check fields indicate one or more fields which should be compared with a threshold value. For example, the registration status (e.g., active, registered, or current (hereinafter these terms may be collectively referred to as active unless the context indicates otherwise)) may indicate an active registration while other terms such as expired, deceased, suspended, revoked, inactive, surrendered, and rescinded (hereinafter these terms may be collectively referred to as inactive unless the context indicates otherwise) may indicate an inactive registration status of the identified user. Thus, if it is determined that the registration of the identified user matches any acceptable term such as active, registered, or current (although other terms are also envisioned), the system generates the MI to so indicate. For example, the system generates the MI to include a corresponding indication of confidence, e.g., one. Similarly, if the system determines (e.g., using any suitable method such as a free-form text search method, etc.) that the registration status of the identified user is not active (e.g., which may occur when the registration status of the identified user matches any undesirable term such as any of the terms such as not-registered, not current, deceased, suspended, revoked, inactive, surrendered, or rescinded (although other terms are also envisioned)), the system generates the MI to indicate such (e.g., a zero to indicate a low degree of confidence) and may flag this MI to draw attention during later processing. One or more fields of the MI may then be weighted as described supra.

Although the identified user may have an MI which is assigned a low value to indicate a low degree of confidence, the user may still be permitted to perform certain tasks. For example, with regard to information such as registration status, if this status is determined to be not active (e.g., inactive), the system may allow the identified user to register but may not allow them to practice until the registration status is changed to an acceptable status such as active. Thus, during each login, the system determines a use for the login such as to register, to review or change/update information, and/or to conduct an encounter with a patient, for HCPs. Each of these uses may have different accessibility rules associated therewith. Further, each type of user (e.g., HCP, HCW, patient, etc.) may have different accessibility rules associated therewith. For example, a patient may register and/or update patient information (PAI) absent certain information such as payment information but may not conduct an encounter nor perform tasks which may result in an immediate or scheduled appointment. These accessibility rules may be defined in an accessibility table and may be stored in memory.

In one embodiment, information is obtained that is used to automatically tune filter gains. For example, the process employs a learning function to learn about current risks with regard to identity fraud and updates system settings accordingly. For example, if it is determined that a certain database was hacked, this source is removed from querying and/or may a lower weight is assigned to information obtained from this source. If it is determined that a source of information (e.g., license information and facial image information etc.) was determined to be hacked, the system removes this source from querying to enhance reliability and security.

Once the filtering is complete (step 741), the it is checked whether user identity is sufficiently validated (step 743). If it is determined that the ID check of the user is valid, the process continues to authorization (step 736). Otherwise, validation using human assistance is performed (step 745).

An ID check of the identified user may be found to be validated when an MI score for one or more required fields either separately and/or combined, depending upon system settings, is equal to or greater than one or more corresponding thresholds such as a corresponding MI Threshold value (MI_{Thresh}). For example, a combined score for all or select MI fields is calculated and compared to a corresponding MI Threshold value (MI_{Thresh}). If the total MI score for each of the corresponding MI field(s) is greater than or equal to MI_{Thresh}, the ID check of the identified user is validated. Otherwise, the ID check of the identified user does not pass. Depending on the implementation, the fields may be chosen by the system (e.g., as required fields with other fields considered non-required fields). The system may then ignore the MI field(s) which are not selected (at least until selected) when for example, determining a combined score, etc. as described infra.

In one embodiment, the MI threshold value(s) (MI_{Thresh}) are set to an integer (or other value) in accordance with predetermined system settings (e.g., a default setting, etc.) or are set to a value equal to a number of fields in a corresponding MI.

When checking a plurality of fields of the MI and assuming the number of fields in MI is equal to F_{MI}, then the value of a corresponding MI threshold value MI_{Thresh} is set equal to a CF_{Thresh}*F_{MI}, where CF_{Thresh} is a confidence factor that, in the present example, may be assumed to be equal to 1 for high confidence and may be set to lower values for a lower confidence factor. Thus, in the present example, MI_{Thresh}=1 and assuming that CF_{Thresh}=1, then if the MI has 6 normalized fields (e.g., MI=0, 0.5, 0, 1, 1, 1), MI_{Thresh}=F_{MI}=6 and if the MI has 9 normalized fields (e.g., MI=1, 0, 1, 0, 1, 1, 1, 0, 0), an MI threshold is set to 9. Thus, for the above example of MI=(0, 0.5, 0, 1, 1, 1), MI_{Thresh}=F_{MI}=6, then the total score of MI=0+0.5+0+1+1+1=3.5 obtained is less than MI_{Thresh}=6 resulting in a no go. Thus, it is determined that the MI score for each of the MI fields is not greater than or equal to MI_{Thresh} and thus the ID check of the user does not pass.

In one embodiment, the MI fields are divided into two or more groups such as selected (e.g., required) and non-required field groups each of which may include at least one field. For example, using the above example, of MI=(0, 0.5, 0, 1, 1, 1), the first three fields for the selected field group are selected and the other fields ignored. Thus, a temporary MI′=(0, 0.5, 0) is processed as described supra. This is done for one or more groups of fields within the original MI. Other values may be set such as CF_{Thresh}, F_{MI}, and/or MI_{Thresh} as discussed supra and the ID check decision made using MI′ for MI as discussed supra.

In one embodiment, human assisted validation is performed (step 745). A GUI that includes one or more portions of the sensor information, the VSI, and the MI is rendered on a display to inform an operator of information related to the identified user such as name, facial information (used during a video conference) and/or other information. For example, the GUI may include information related to fields of the MI that were below a threshold confidence factor. If a user's address was incorrect, this is flagged and similar fields of information (e.g., all addresses in the current example) on record for the identified user is included in the GUI for the convenience of the operator.

If the identified user moved from one address to another, this is brought to the attention of the operator. Similarly, if the identified user is a physician with an inactive license status, this information is flagged and included in the GUI. A communication channel may be established between the operator and the identified user so that text, audio, and/or video information may be exchanged. The GUI may further include available information fields and sources of information so the operator can quickly and conveniently confirm information. The GUI may include an override, which overrides selected information (e.g., overrideable information which may be predefined and stored memory) such as address information, facial image information, etc. Thus, if the user changes address due to a move and/or shaves his beard to change facial characteristics, the system provides a method to override. Other information such as license status (e.g., if not active) may be non-overrideable unless authorized by a user with the proper authorization. Thus, during the conversation with the identified user, the operator may request additional information from the user and/or databases and be provided with an option to pass an ID check of the user. Further, the operator may update and/or override information related to the identified user and the system stores these updates in memory. Accordingly, when the identified user attempts to login at a future date, the override information is checked to avoid repeating the failure of the ID check for the same reason(s).

Once identity is validated, users are then authorized to interact with the system (step 736). At this point, the identified user is considered to have passed the ID check. A GUI notifies the user that they have passed the ID check and are authorized to use the system in accordance with system rights information (SRI) for the user. The SRI may set forth rights (e.g., authorizations, etc.) for each user in accordance with group and/or subgroup settings for a group and/or subgroup to which the identified user belongs and/or individual settings as may be stored in PAI. The SRI is stored in memory in any suitable format. The SRI sets forth rights and privileges of the identified user during at least the present session and/or future sessions. Accordingly, the system determines a group to which associate the present user such as: a provider, worker, patient group 738, taxi driver/realtor group 740, home service group 742, care team group 744, and/or an identification of other personnel group 746, and sets system rights information (SRI) accordingly for the user's particular group and/or subgroup.

### Healthcare Provider Workflow

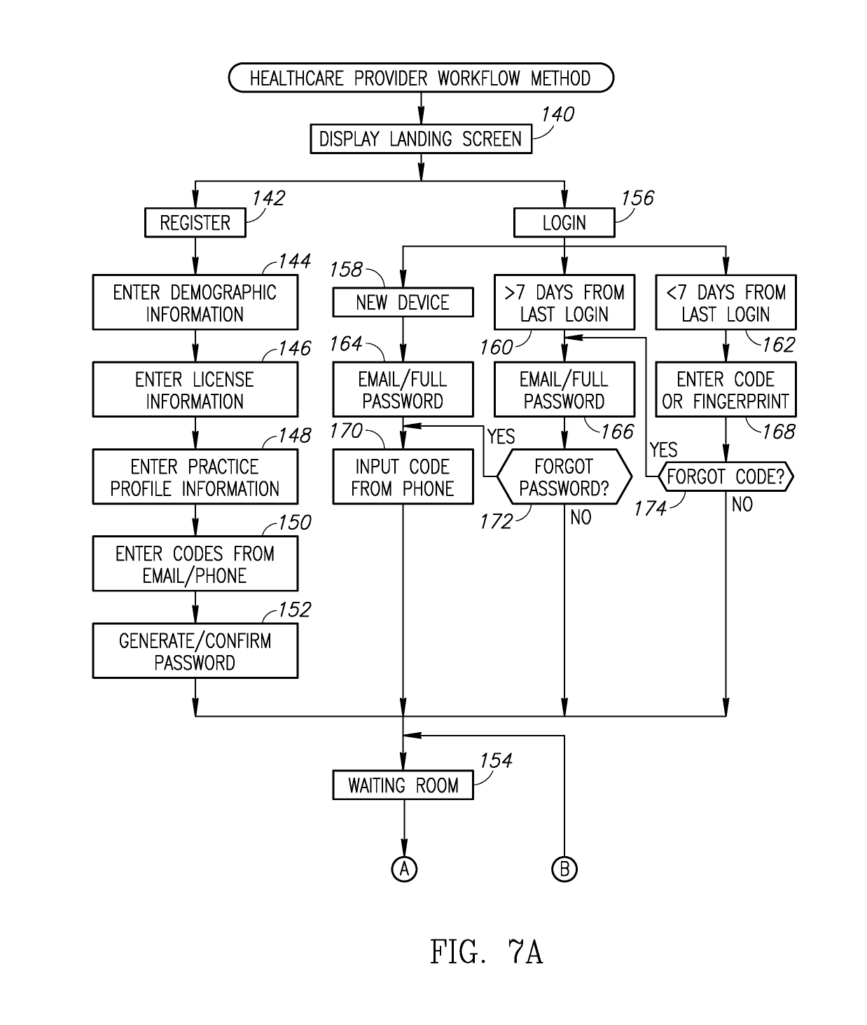

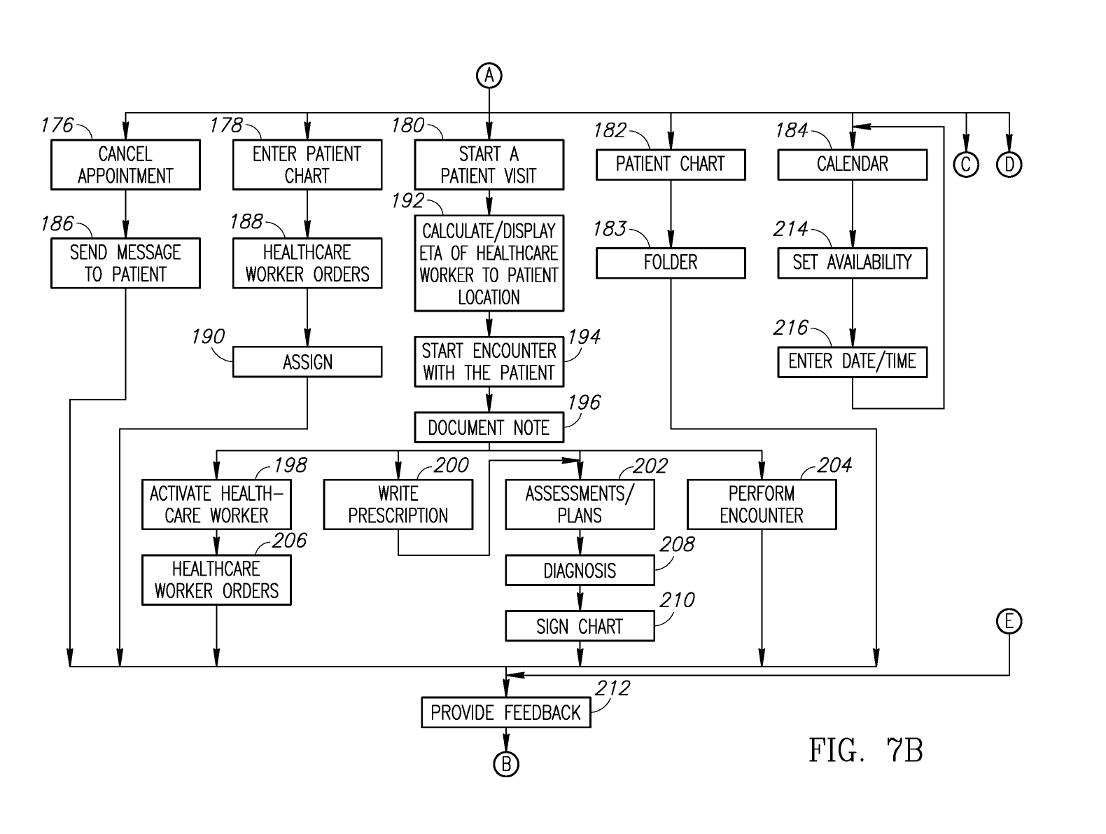

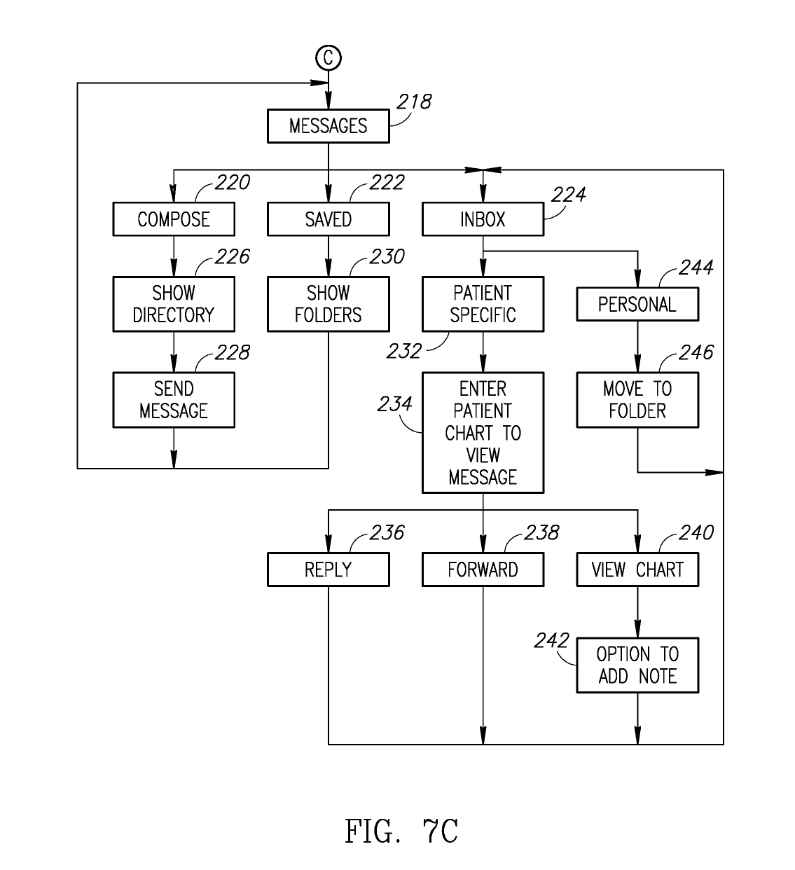

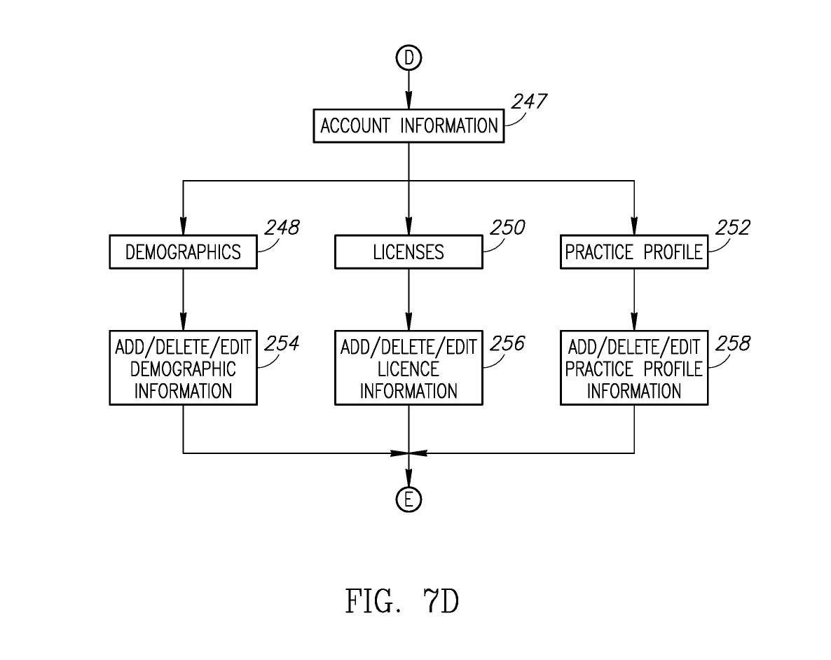

A flow diagram illustrating an example healthcare provider workflow method of the present invention is shown in FIGS. 7A, 7B, 7C, and 7D. First, a landing screen is displayed on the provider's computing device (step 140). The provider then selects to either register (step 142) as a new provider or login (step 156) as a previously registered provider. To log in, the provider selects one of three options. The first option is to log in from a new device (step 158). The provider enters their full email and password (step 164) and input a code generated by the system that is sent to the provider's computing device such as a smartphone or tablet (step 170). The second option is to log in more than seven days from the last login (step 160). The provider must enter their full email and password (step 166). If they forgot their password (step 172), they must input a code generated by the system that is sent to the provider's computing device such as a smartphone or tablet (step 170). The third option is to log in less than seven days from the last login (step 162). In this case, the provider is prompted to enter a code or to scan their fingerprint (step 168). If they forgot their code (step 174), they must enter their full email and password (step 166) and continue with step 172.

To register (step 142) with the system, the provider enters demographic information (e.g., name, home address, work address, gender, ID number such as social security number (SSN), date of birth, etc. (step 144). The provider then enters license information (e.g., driver's, Drug Enforcement Agency (DEA) number, state licenses, etc.) (step 146) and practice profile information (e.g., email, work phone number, home phone number, cellphone number, login ID, profile photo, screen name, employee ID, spoken languages, insurance plans accepted, group practice name, national provider ID (NPI), etc.) (step 148). Once the ID and licenses of the provider have been validated and verified, the system sends an email or text message to the new user. The provider then enters the code(s) received (step 150). A passcode and/or touch ID on mobile devices is created for easy access. The provider then enters a password or the system generates one and the provider confirms (step 152).

Note that during the registration process, the provider's identify is validated and verified. This can be achieved using any number of techniques and processes such as input a code sent via email, inputting a code sent via test messaging, facial recognition from a picture, fingerprint, touch ID, using data retrieved from public, private, criminal and government databases, e.g., driver license information.

Note also that a new healthcare provider account can be created manually or automatically. The provider or other staff member can perform a manual creation. Alternatively, the account can be created by an automatic load from an external system (e.g., EPIC or other practice management system).

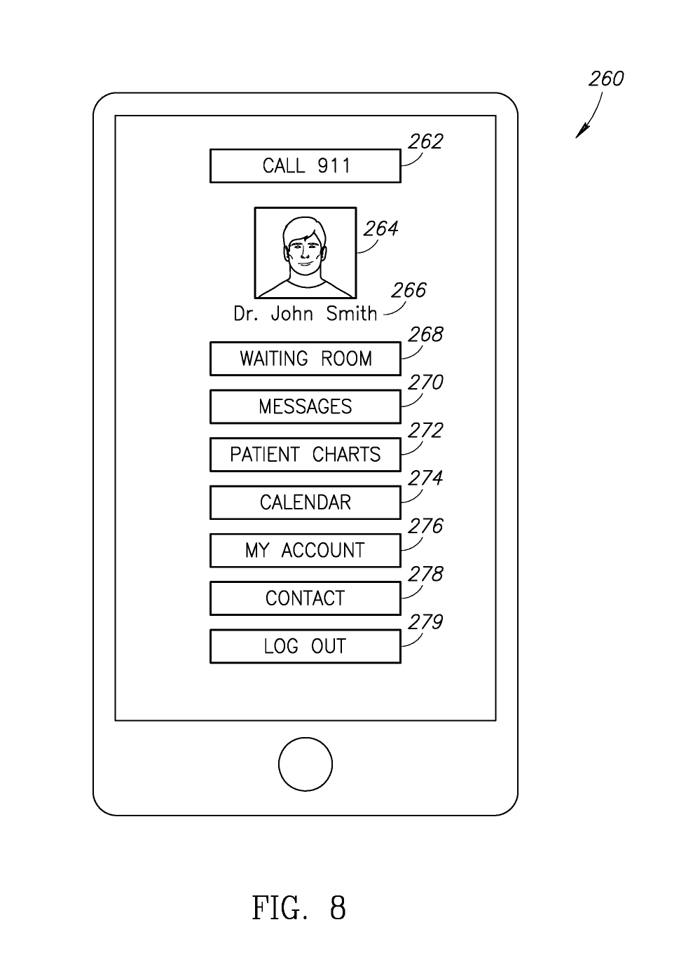

Once registration or login is complete, the provider is placed on the waiting room landing screen 154 such as shown in FIG. 8. Via example screen 260, the provider can call emergency services such as 911 (262), see their photo 264 and name 266, enter the waiting room 268, receive and send messages 270, view and edit patient charts 272, see and edit calendars 274, edit their account 276, contact system administration 278 and log out from the system 279.

Appointments previously made with a patient can be canceled (step 176). When an appointment is canceled, a message is sent to the patient with an option to reschedule (step 186).

The provider can enter a patient's medical chart (step 178) and add, delete and edit the patient's record. The provider can also generate orders or tasks to be performed by one or more healthcare workers at the patient's location, for example (step 188). Once generated, the provider assigns one or more orders or tasks to a healthcare worker (step 190).

In one embodiment, a provider or worker can generate a clinical report that can include any or all of the following related to the encounter: summary, clinical notes, medications prescribed, patient instructions, images, medical and social history, and medications and allergies.

The provider can also initiate a patient visit (i.e. encounter) whereby a healthcare worker may be dispatched to the patient's location (step 180). The system calculates and displays the estimated time of arrival (ETA) to the patient's location of those healthcare workers within a certain radius thereof (step 192). The provider then starts an encounter with the patient (step 194). Note that the encounter is typically a video encounter but can take other forms, e.g., text session, etc.). The provider can record and enter any document notes into the patient's record.

In one embodiment, the data elements captured during an encounter include any or all of the following: date, provider name, reason for the encounter (e.g., complaint), primary diagnosis, clinical notes, images, medication(s) prescribed, patient instructions, and signature date.

At this point, the provider can activate a healthcare worker (step 198) whereby the worker is dispatched to the patient location. The provider can also issue any number of orders and/or tasks to be performed by the worker (step 206).

The provider can write one or more prescriptions at any time during the encounter (step 200). The prescriptions may be processed and sent to a pharmacy using a third party software tool such as MDToolbox-Rx as described in more detail infra.

Any assessments and plans are recorded by the provider into the patient record (step 202). Any diagnosis is also recorded into the patient record (step 208). Once the diagnosis is records, the provider electronically signs the patient's chart (step 210).

During the encounter (step 204), the provider can request the worker who is at the patient's location to perform a physical examination of the patient possibly using any type of sensor, perform any procedures on the patient, etc. In essence, the worker with the patient acts as the eyes, ears and touch of the provider.

The provider is also provided with messaging services (step 218) including composing and reading messages. When composing a message (step 220), the provider selects a recipient from a directory of recipients (step 226) and the system sends the message (step 228). Saved messages can be accessed (step 222) and are shown within selected folders (step 230). An inbox (step 224) holds both received patient related messages (step 232) as well as personal messages (step 244). Personal messages are moved to a personal folder (step 246), while patient related messages are displayed along with the patient's chart (step 234). From there, the provider can reply to the message (step 236), forward the message (step 238) or view the chart (step 240) and optionally add a note thereto (step 242).

The provider can also access account information (step 247) including demographic information (step 248) that can be edited (step 254), license information (step 250) that can be edited (step 256) and practice profile information (step 252) that can be edited (step 258).

In one embodiment, after providing feedback (step 212), the provider is taken back to the waiting room landing screen (step 154).

At any point during an encounter, the provider may decide that medication is required for the treatment of the patient. The system provides the capability for the provider to write any number of electronic prescriptions before terminating the encounter or discharging the patient. In one embodiment, prescription writing is handled using third party software tools such as MDToolbox-Rx. Alternatively, prescription writing can be implemented entirely within the system platform.

During the encounter, the provider reviews and records key facts about the patient's other medications and drug allergies and indicates whether medication history has been verified before prescribing. Information collected during encounters is recorded in the patient medical record.

Before prescribing any medications during an encounter, the provider the system displays one or more screens that include current medications and allergies. Information about current medications and allergies is also transmitted to the third party tool (e.g., MDToolbox-Rx) to check for contraindications. The provider then selects drugs, doses, selected pharmacy, etc. The system or the third party tool transmits the prescription(s) to the selected pharmacy. If successful, information related to the prescription is returned to the system for storing in a database. Once stored, this information can be viewed by providers, workers and patients. Note that renewals are handled in similar fashion to new prescriptions. Providers can optionally include prescription information with patient discharge instructions.

### Healthcare Worker Workflow

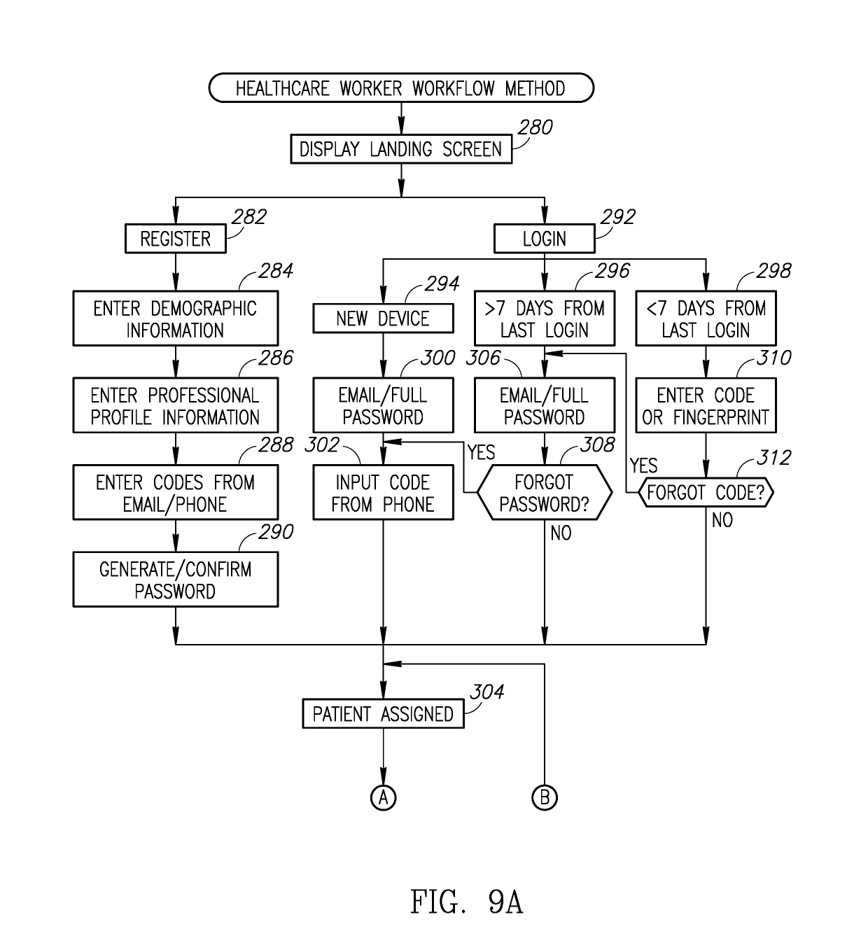

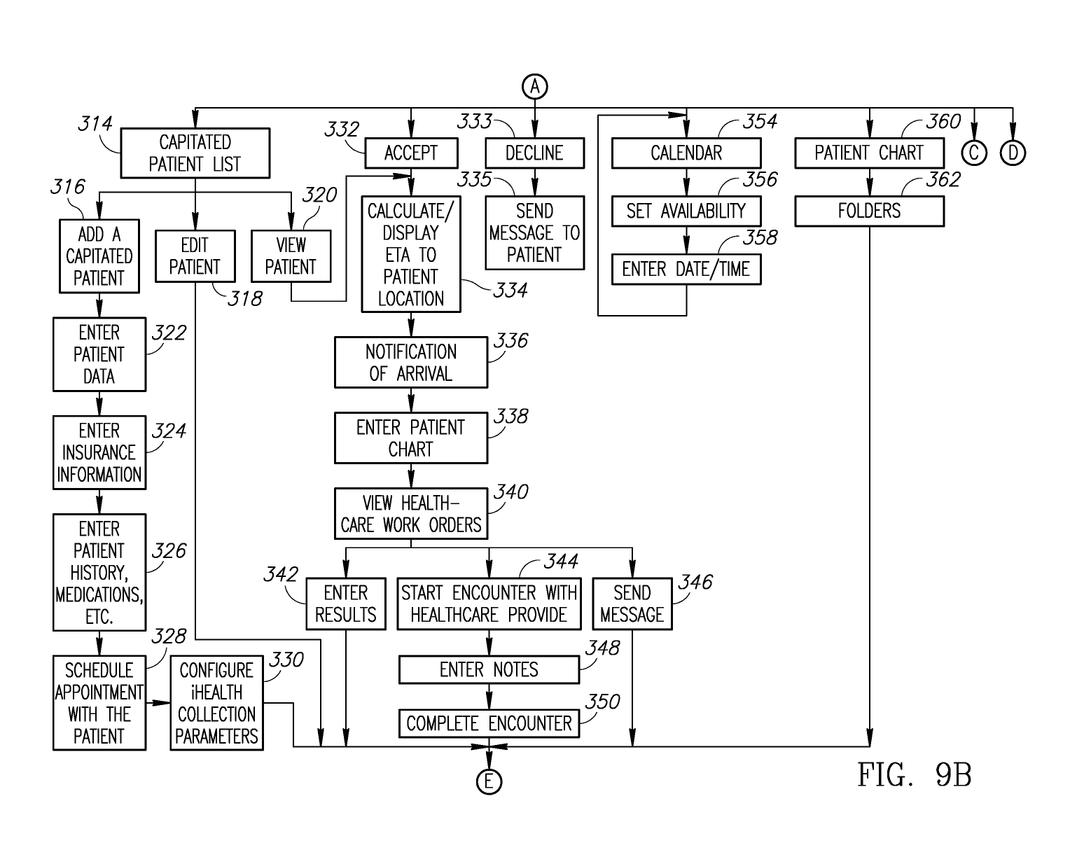

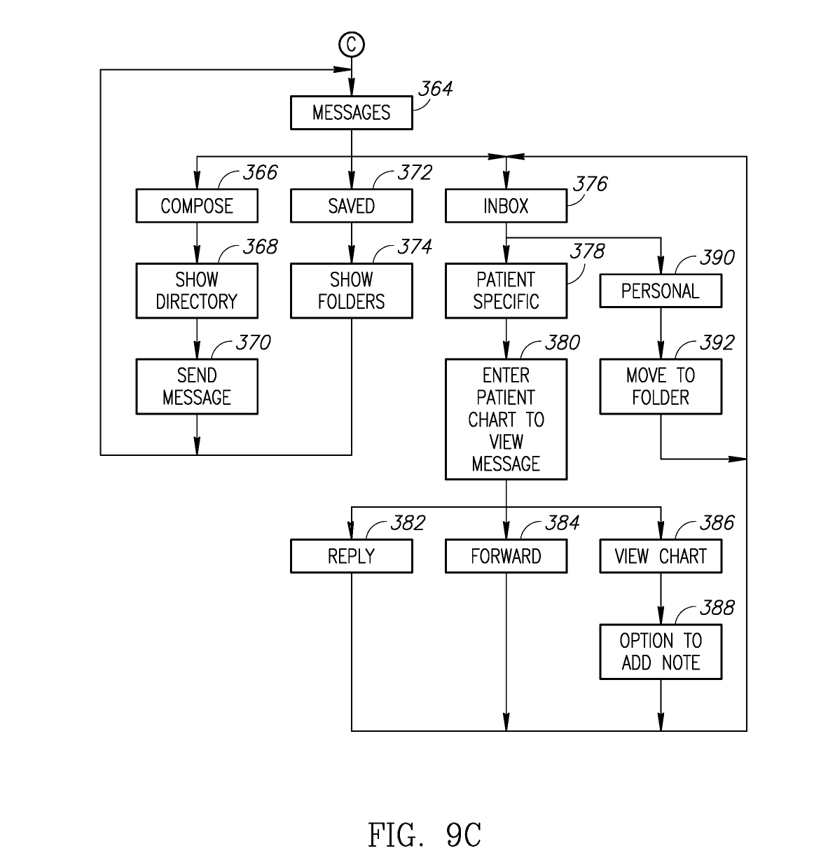

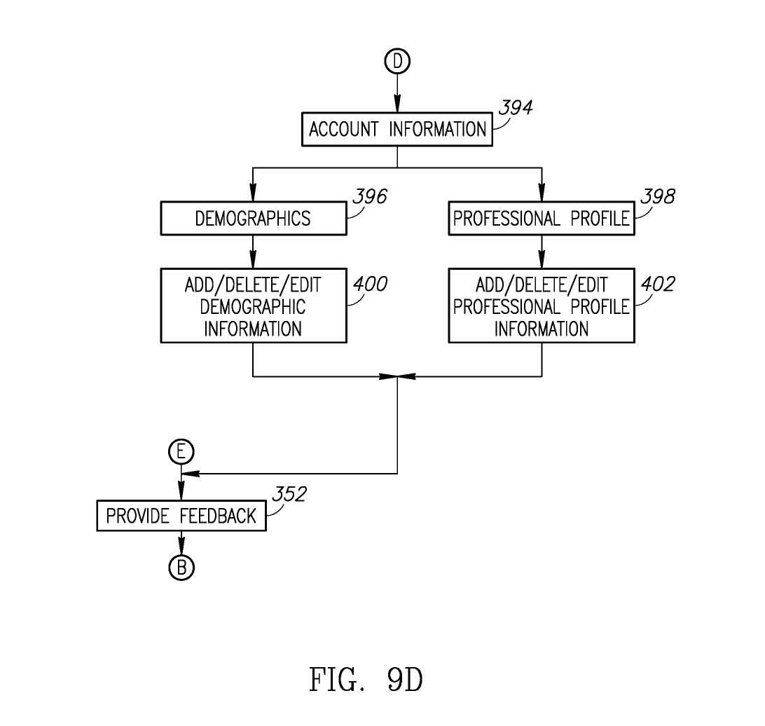

A flow diagram illustrating an example healthcare worker workflow method of the present invention is shown in FIGS. 9A, 9B, 9C, and 9D. First, a landing screen is displayed on the worker's computing device (step 280). The worker then selects to either register (step 282) as a new worker or login (step 292) as a previously registered worker. To log in, the worker selects one of three options. The first option is to log in from a new device (step 294). The worker enters their full email and password (step 300) and input a code generated by the system that is sent to the worker's computing device such as a smartphone or tablet (step 302). The second option is to log in more than seven days from the last login (step 296). The worker must enter their full email and password (step 306). If they forgot their password (step 308), they must input a code generated by the system that is sent to the worker's computing device such as a smartphone or tablet (step 302). The third option is to log in less than seven days from the last login (step 298). In this case, the worker is prompted to enter a code or to scan their fingerprint (step 310). If they forgot their code (step 312), they must enter their full email and password (step 306) and continue with step 308.

To register with the system (step 282), the worker enters demographic information (e.g., name, home address, work address, gender, ID number such as social security number (SSN), date of birth, etc. (step 284). The provider then enters practice profile information (e.g., email, clinical credentials (e.g., RN, CNA, EMT, MA, etc.), license information (e.g., driver's, state licenses, etc.), work phone number, home phone number, cellphone number, login ID, employee ID, profile photo, screen name, spoken languages, insurance plans accepted, group practice name, national provider ID (NPI), etc.) (step 286). Once the ID and clinical credentials of the worker have been validated and verified, the system sends an email or text message to the new user. The worker then enters the code(s) received in the email or text message (step 288). A passcode and/or touch ID on mobile devices is created for easy access. The worker then enters a password or the system generates one and the worker confirms (step 290).

Note that during the registration process, the worker's identify is validated and verified as described supra. Note also that a new healthcare worker account can be created manually or automatically as described supra.

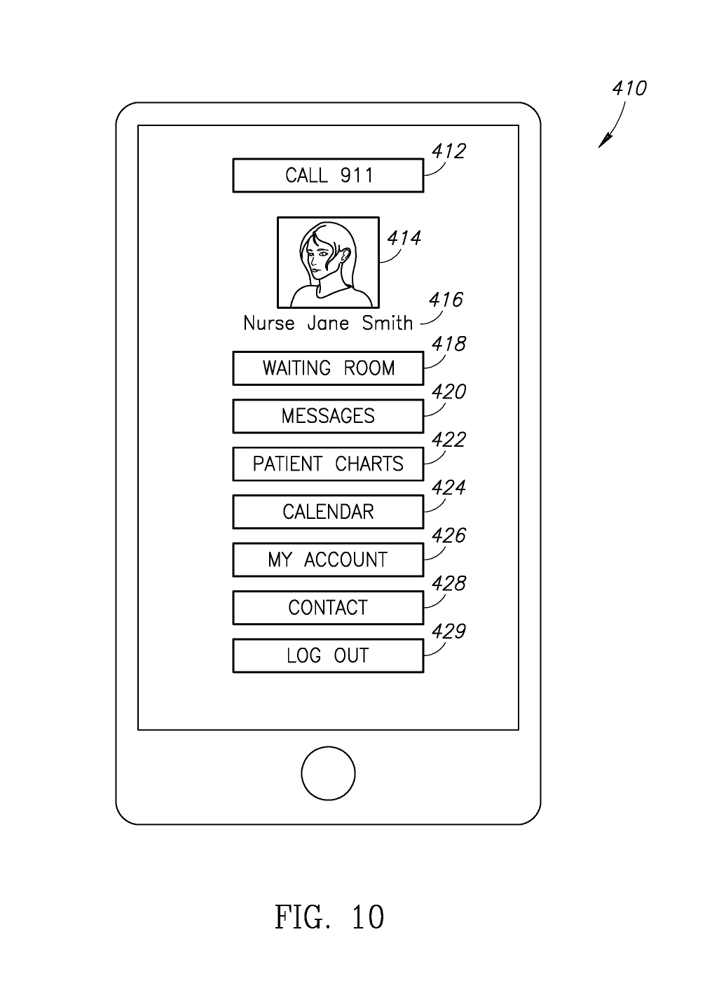

Once registration or login is complete, the worker is placed on the patients assigned landing screen 304 such as shown in FIG. 10. Via example screen 410, the worker can call emergency services such as 911 (412), see their photo 414 and name 416, enter the waiting room 418, receive and send messages 420, view and edit patient charts 422, see and edit calendars 424, edit their account 426, contact system administration 428 and log out from the system 429.

In addition, once logged into the system, the worker's location is tracked by GPS in their mobile computing device or other means. This enables the time to the patient's location to be estimated so the closest worker can be dispatched to the patient. Knowledge of the location of the worker also permits the tracking of resources in the field.

The worker can view the capitated patients list (step 314). Note that capitation is a payment arrangement for healthcare providers that pays a provider a set amount for each enrolled patient assigned to them, per period of time, whether or not that patient seeks care. The worker can then add a patient (step 316), edit a patient (step 318) or view a patient (step 320). To add a patient, the worker enters the patient data (step 322), insurance information (step 324), and patient history, medications, etc. (step 326). An appointment can then be scheduled with the patient (step 328) and any iHealth collection parameters configured (e.g., blood pressure, sleep trackers, glucometers, scales, pulse oximeters, etc.) (step 330).

For patients that are assigned to the worker by a provider, the worker has the option of either accepting (step 332) or declining (step 333) the assignment. If the worker declines the assignment, a message is sent to the patient and the provider (step 335). If the worker accepts the assignment, the system calculates and displays the ETA to the location of the patient based on the current location of the worker (step 334). The worker is displayed an optimum travel route to the patient along with the ETA. The worker can communicate with the patient and/or provider via messaging. One or more messages may be automatically generated and sent to the patient and/or the provider indicating the current location and en route status information of the worker.

A notification of the arrival of the worker at the patient location is generated upon arrival (step 336). The worker then enters the patient chart for viewing and editing (step 338) and also views any worker orders or tasks entered by the provider (step 340). Optionally, the patient's identity may be verified before the visit proceeds further. The worker then performs any orders or tasks assigned by the provider and enters results into the patient chart (step 342). Examples include obtaining lab samples, dropping off medications, taking x-rays, repairing a laceration, etc.

During the encounter, the provider can request the worker who at the patient's location to perform a physical examination of the patient possibly using any type of sensor (e.g., stethoscope, otoscope, etc.), perform any procedures on the patient, etc. In essence, the worker acts as the eyes, ears and touch of the provider.

The worker can start an encounter with the provider or reconnect the patient with the provider (e.g., video) (step 344), enter notes into the chart (step 348) and complete the encounter (step 350). The worker can also compose and send messages to other workers, providers or other patients assigned to the worker (step 346).

In addition, the worker has access to one or more calendars (step 354) where they can set their availability (step 356) and enter dates/times, etc. (step 358).

Workers can also view charts for patients assigned to them (step 360) as well as any information contained in one or more folders (step 362).

The worker is also provided with messaging services (step 364) including composing and reading messages. When composing a message (step 366), the worker selects a recipient from a directory of recipients (step 368) and the system sends the message (step 370). Saved messages can be accessed (step 372) and are shown within selected folders (step 374). An inbox (step 376) holds both received patient related messages (step 378) as well as personal messages (step 390). Personal messages are moved to a personal folder (step 392), while patient related messages are displayed along with the patient's chart (step 380). From there, the worker can reply to the message (step 382), forward the message (step 384) or view the chart (step 386) and optionally add a note thereto (step 388).

The worker can also access account information (step 394) including demographic information (step 396) that can be edited (step 400) and professional profile information (step 398) that can be edited (step 402).

In one embodiment, after providing feedback (step 352), the worker is taken back to the patients assigned landing screen (step 304).

Once the encounter and visit are complete, the worker indicates this in the system. The worker then becomes active again and can be assigned to another patient. Note that in one embodiment, the worker can be assigned another patient during a visit with a patient. The worker can accept or decline each new patient assignment while engaged in another appointment. The worker can view their patient queue at any time.

Note that in one embodiment, in-field workers can initiate an encounter with a provider. Patients may have a planned visit by the healthcare worker or be located in a facility such as a nursing home, assisted living center, etc. where the worker is already present. In this case, if the healthcare worker determines that the patient needs to be evaluated by the provider, the worker can connect with the provider either through either an immediate or scheduled appointment using the mechanisms described supra. The worker can then assist the provider with a more thorough examination using any number of sensors such as a stethoscope, otoscope, blood pressure monitor, etc. During the encounter, the provider can direct the worker to take any lab or other tests that may help with a more complete encounter.

### Patient Workflow

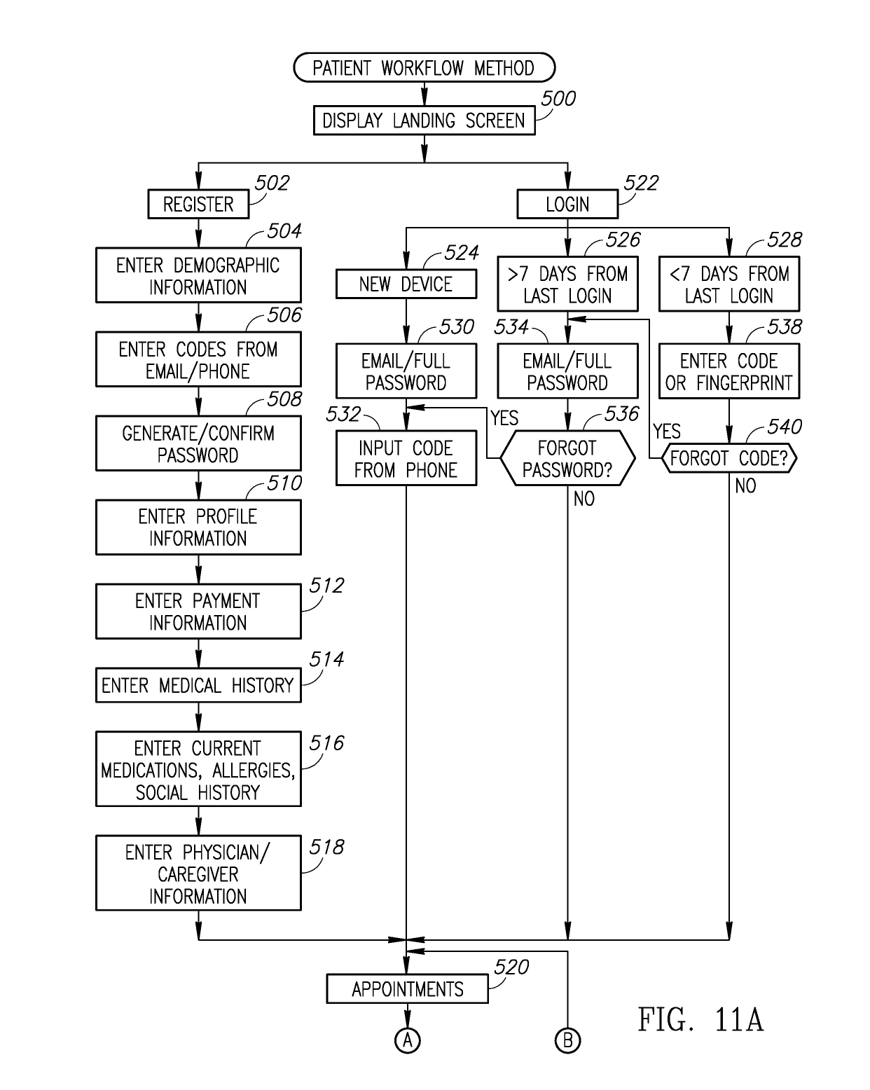

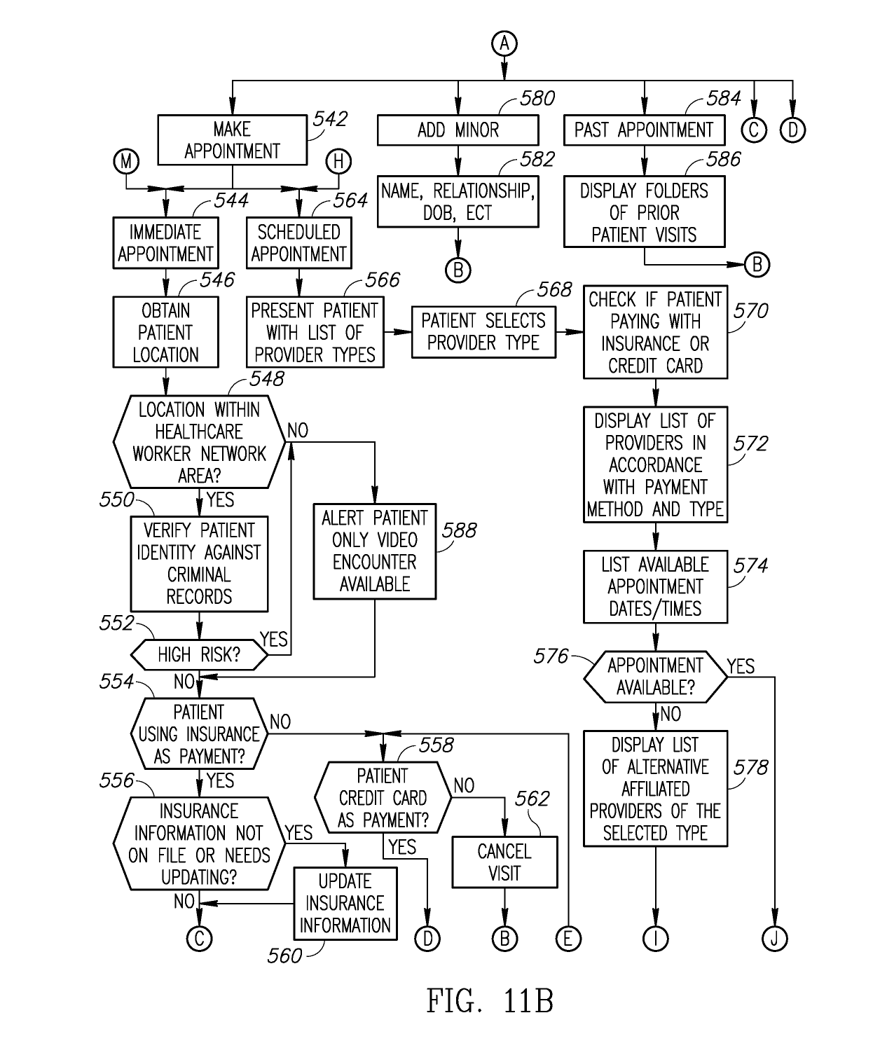

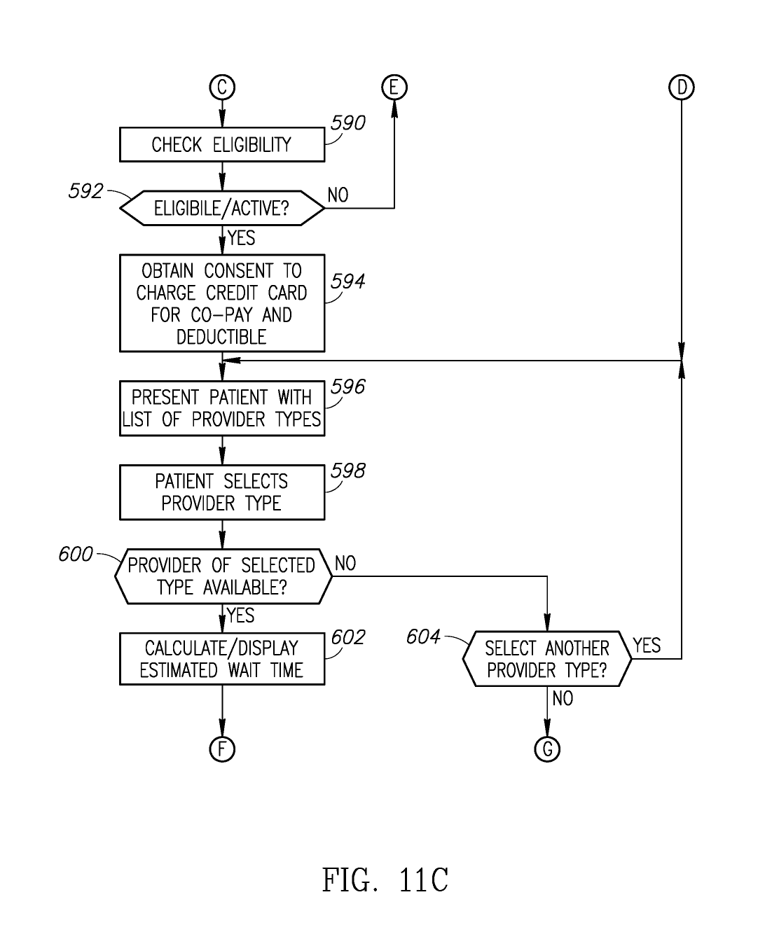

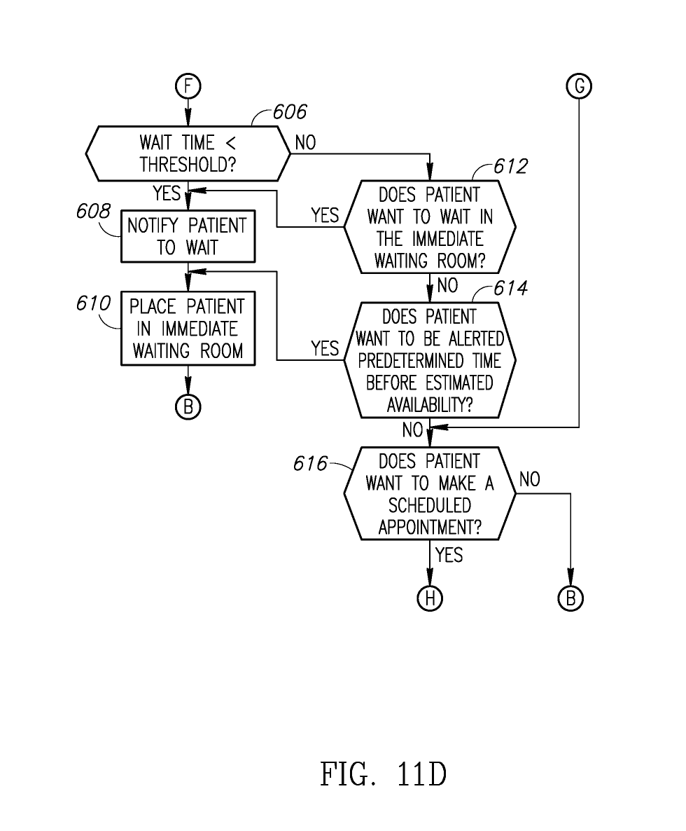

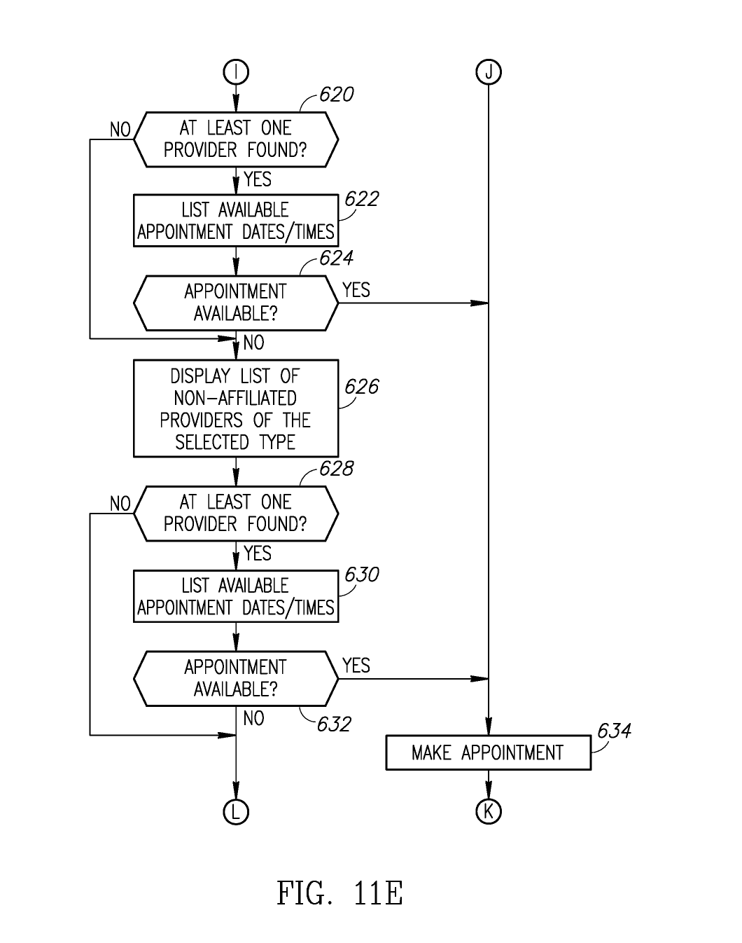

A flow diagram illustrating an example patient workflow method of the present invention is shown in FIGS. 11A, 11B, 11C, 11D, 11E, and 11F. First, a landing screen is displayed on the patient's computing device (step 500). The patient then selects to either register (step 502) as a new patient or login (step 522) as a previously registered patient. To log in, the patient selects one of three options. The first option is to log in from a new device (step 522). The patient enters their full email and password (step 530) and inputs a code generated by the system that is sent to the patient's computing device such as a smartphone or tablet (step 532). The second option is to log in more than seven days from the last login (step 526). The patient enters their full email and password (step 534). If they forgot their password (step 536), they must input a code generated by the system that is sent to the worker's computing device such as a smartphone or tablet (step 532). The third option is to log in less than seven days from the last login (step 528). In this case, the patient is prompted to enter a code or to scan their fingerprint (step 538). If they forgot their code (step 540), they must enter their full email and password (step 534) and continue with step 536.

To register (step 502) with the system, the patient enters demographic information (e.g., name, home address, work address, gender, ID number such as social security number (SSN), date of birth, etc. (step 504). The system sends an email or text message to the new patient. The patient then enters the code(s) received in the email or text message (step 506). A passcode and/or touch ID on mobile devices is created for easy access. The worker then enters a password or the system generates one and the patient confirms (step 508). The patient then enters profile information (e.g., login ID or username, email, license information (e.g., driver's, etc.), work phone number, home phone number, cellphone number, profile photo, screen name, spoken languages, etc.) (step 286).

The patient then enters payment information such as primary and secondary insurance, including insurance company, policy number, member number, front and back insurance ID photos, etc. (step 512). Medical history information is then entered (step 514) followed by current medication, any allergies, social history (e.g., drugs, smoking), etc. (step 516).

Physician and care team information is then entered, e.g., care team member names (i.e. family, friends, etc.), contact phone numbers, relation to the patient, etc. (step 518). Care team members have a registered account with the system to permit them to access information on the patient they are linked to. When logged in, they have the ability to toggle between their own account and that of their ‘patient.’ They have access to that patient's data based on what is shared, e.g., full access or choose which features they are given access to. For example, patients can share and edit appointments, share patient instructions (e.g., discharge instructions), share and edit medications and allergies, medical history, allow communications with the medical team, share disease management data, permit reception of notifications, view test results, pay bills, view and edit registration and join video encounters.

Note that accounts for minors can also be created and setup. Minor accounts can be linked to a legal guardian or parent's account. Note also that during the registration process, the patient's identify is validated and verified as described supra. Note further that a new patient account can be created manually or automatically as described supra.

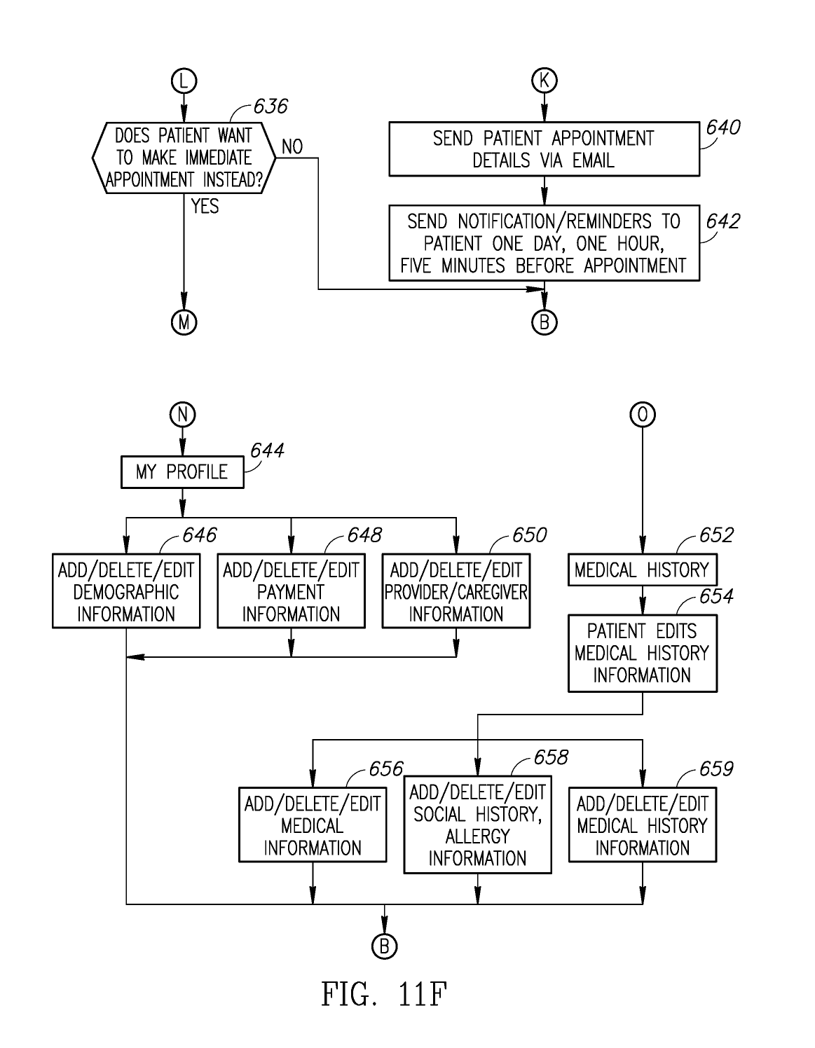

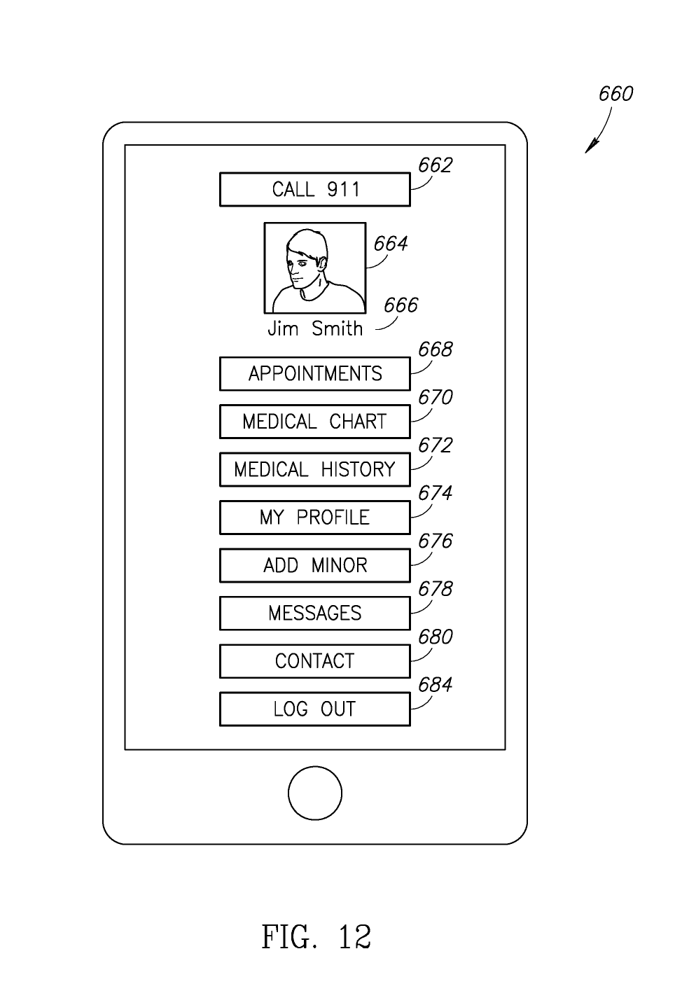

Once registration or login is complete, the patient is placed on the appointments landing screen 520 such as shown in FIG. 12. Via example screen 660, the patient can call emergency services such as 911 (662), see their photo 664 and name 666, view and make appointments 668, view their medical chart 670, view and edit their medical history 672, view and edit their profile 674, create a minor account 676, receive and send messages 678, contact system administration 680 and log out from the system 684.

Using the system, patients can make appointments with providers (step 542). In one embodiment, two types of appointments are possible: immediate appointments and scheduled appointments. To make an immediate appointment (step 544), the location of the patient is first acquired (step 546). It is then checked whether the patient's location is within a worker's network area (step 548). If it is not, then the patient is alerted that only an encounter (e.g., video) is available and that a worker cannot be dispatched to their location (step 588).

If the location is within a worker's network area, the patient is then verified against criminal records databases (step 550) as it is not desired to send a worker to a patient that may be high risk. If the patient is high risk, then the patient is alerted that only an encounter (e.g., video) is available (step 588). If the patient is not high risk, it is then determined whether the patient is using insurance as payment (step 554). If so, it is checked whether the insurance information is on file and whether it needs updating (step 556). If the information is not on file or needs updating, then the patient is offered the option to update their insurance information (step 560). If the information is on file and no updating is needed, patient eligibility is checked with their insurance (step 590). If they are active/eligible (step 592), consent is obtained from the patient to charge their credit card for any copayment and deductible charges (step 594). If they are not active or not eligible, the method continues with credit card payment step 558.

Once consent is obtained, the patient is then presented with a list of provider specialty types (step 596). The patient then selects a provider specialty type (step 598). If a provider of the selected type is available (step 600), then the estimated wait time is calculated and displayed to the patient (step 602). If the wait time is less than a threshold (step 606), the patient is notified to wait (step 608), the patient is placed in the immediate waiting room (step 610) and the patient is returned to the appointment landing page (step 520). When the provider is ready, the patient receives a notification to enter the encounter (e.g., video) for their appointment.

If the patient is not using insurance as payment (step 554), then it is checked whether the patient is using a credit card as payment (step 558). If not, the appointment process is canceled and the patient is returned to the appointment landing page (step 520). Otherwise, the patient is presented with a list of provider specialty types (step 596) and the method continues as described supra.

If a provider of the selected specialty type is not available (step 600), the patient is offered the option of selecting a different type (step 604). If they choose not to, then the patient is offered the option of making a scheduled appointment and continues with step 616. If they want to choose another type, the method continues with step 596.

If the wait time is greater than or equal to the threshold (step 606), then the system asks the patient if they want to wait in the immediate waiting room (step 612). If so, the method continues with step 608. Otherwise, the patient is asked if they want to be alerted a predetermined time before the estimated availability (step 614). If so, they are placed in the immediate waiting room (step 610) and they are notified before the appointment. Otherwise, the patient is given the option to make a scheduled appointment (step 616). If they choose not to, the method returns to the appointment landing page (step 520). If they do, the method continues with making a scheduled appointment (step 564).

To make a scheduled appointment, the patient is first presented with a list of provider specialty types (step 566). The patient selects a provider type (step 568) and the system checks whether the patient is paying with insurance or credit card (step 570). The system then displays a list of providers in accordance with the patient's insurance and type selection (step 572). Alternatively, the patient is given the option to view all providers including those that do not accept the patient's insurance.

A list of available dates/times that provider or multiple providers is available is then displayed (step 574). If an appointment is available at the date/time selected by the patient (step 576), an appointment is made (step 634). Appointment details are then sent via email or other means to the patient (step 640). In addition, notifications and reminders are sent to the patient at certain time before the appointment, such as one day, one hour, and five minutes (step 642). The patient then returns to the appointment landing page (step 520).

If the desired appointment is not available (step 576), then a list of alternative affiliated providers of the selected type are displayed to the patient (step 578). If no provider is found (step 620), the method continues with step 626 which displays a list of non-affiliated providers and/or providers that do not accept the patient's insurance. If at least one provider is found, the available appointment dates/times are displayed to the patient (step 622). If an appointment at the desired date/time is available (step 624), the appointment is made and the method continues with step 634. Otherwise, a list of non-affiliated providers of the selected type are displayed to the patient (step 626). If no provider is found (step 628), the method continues with step 636 where the option to make an immediate appointment is offered to the patient. If the patient chooses not to make and immediate appointment, then the patient returns to the appointment landing page and continues with step 520. If the patient wishes to make and immediate appointment, the method continues with step 544. If at least one provider is found (step 628), the available appointment dates/times are displayed to the patient (step 630). If an appointment at the desired date/time is available (step 632), the appointment is made and the method continues with step 634. At the time of the appointment, the patient is sent a link and/or notification to enter the encounter room (e.g., video) for the appointment when the chosen provider is ready.

As described supra, the patient can create accounts for minors (step 580). The patient enters the minor's name, relationship, date of birth, etc. (step 582).

Patients can view past appointments (step 584). Folders containing prior patient visits are displayed to the patient (step 586).

The patient can also access profile information (step 644) including demographic information that can be edited (step 646), payment information that can be edited (step 648), and provider/caregiver/care team information that can be edited (step 650).

Patients can also view and update their medical history and those of minors linked to them (step 652). Screens are presented to the patient to edit medical history information (step 654). This includes editing medical information (step 656), editing social history, allergy information (step 658), and editing medical history information (step 659).

### Immediate and Scheduled Waiting Rooms

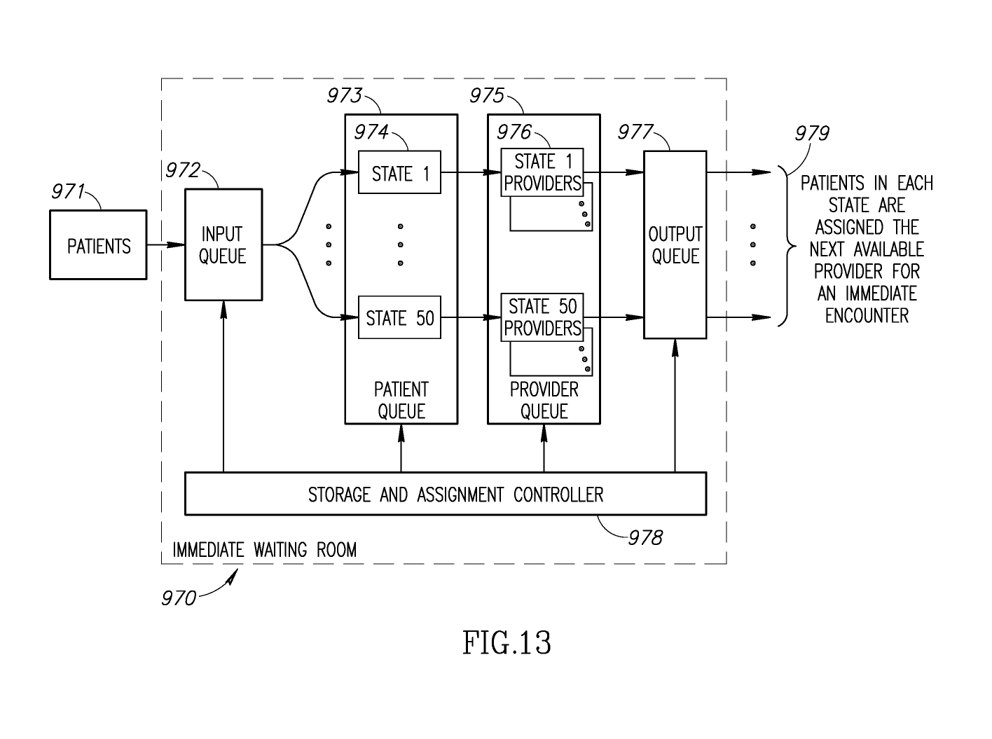

A diagram illustrating an example immediate waiting room in more detail is shown in FIG. 13. The immediate waiting room, generally referenced 970, comprises an input queue 972, patient queue 973, provider queue 975, output queue 977 and storage and assignment controller 978. In operation, patients 971 entering the immediate waiting room are first placed in the input queue 972. Depending on the state where they are located, they are then placed in a state queue 974 corresponding to their particular state. The patients in each state queue are then fed to the queue 976 of a provider in that state. The output queue then feeds patients 979 to the individual providers. The storage and assignment controller 978 is operative to assign patients in each state to the next available provider in that state for an immediate encounter. In this manner, patients do not choose their provider, but rather are assigned to the next available provider in that state.

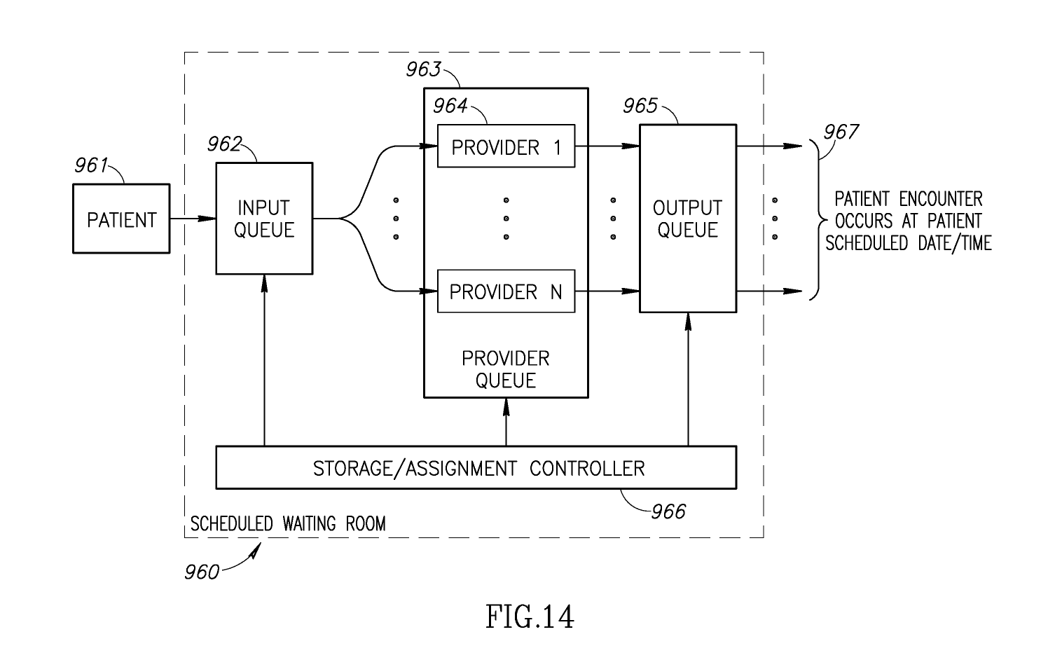

A diagram illustrating an example scheduled waiting room in more detail is shown in FIG. 14. The scheduled waiting room, generally referenced 960, comprises an input queue 962, provider queue 963, output queue 965 and storage and assignment controller 966. In operation, patients 961 entering the scheduled waiting room are first placed in the input queue 962. Depending on the particular provider they scheduled with, they are then placed in a provider queue 964 corresponding to the selected provider. The patients in each provider are then fed to the output queue which then feeds patients 967 to the individual providers. The storage and assignment controller 966 is operative to assign patients to their chosen provider for a scheduled encounter. In this manner, patients choose their provider and an encounter occurs at the patient scheduled date/time.

A diagram illustrating an example mobile device screenshot of patient appointment selection is shown in FIG. 15. In one embodiment, the appointment selection screen includes a GUI 910 including selection items 912 for selecting an appointment type such as, an immediate-type appointment (ITA) 914 and a scheduled type appointment (STA) 916 which may be selected by the patient in accordance with methods described in more detail supra.

Accordingly, the system generates content which informs the patient to, for example, select an immediate appointment or to schedule an appointment at a later time, and may include graphics and/or text suitable to inform a user and/or receive a selection of a desired appointment type from the patient.

In addition, a help and/or guidance button is provided for selection by the user. Guidance selection items such as a back arrow 919 and/or a question mark help icon “?” 918 are provided to assist navigation of displays by a user. If the help icon “?” is selected, the system retrieves assistance information from memory corresponding to the current screen and renders it on the display. For clarity sake, guidance, help, and/or other selection items may not be shown in subsequent screen capture drawings.

A diagram illustrating an example mobile device screenshot of patient healthcare provider selection is shown in FIG. 16. The GUI screenshot, generally referenced 920, is displayed on the UI of a US of the patient. In one embodiment, the GUI comprises a list of modified physician type information (PTI) 922, and an area 924 for a current patient to enter information such as text (e.g., using a text entry area) which is recognized as the patient enters it. The system determines and renders autocompletion data based upon information in the PTI 922 for the convenience of the user. For example, if the user enters the letters PE, the system highlights (and/or changes the order of) a matching physician type such as “Pediatrician” for the convenience of the user. Thus, the system places “Pediatrician” at the top of the list and/or autocompletes the entry in the search box.

The patient then selects a physician specialty type (or types) using any suitable method such as by selecting a menu item, entering text and/or a voice command, etc. and the system enters this information as a selected physician type.

The PTI is stored in memory and includes information related to physicians such as names, qualifications, licensure (e.g., state, term, current license status, medical school, residency, etc.), patient acceptance, patient age range, and practice type (e.g., cardiology, ob/gyn, etc.).

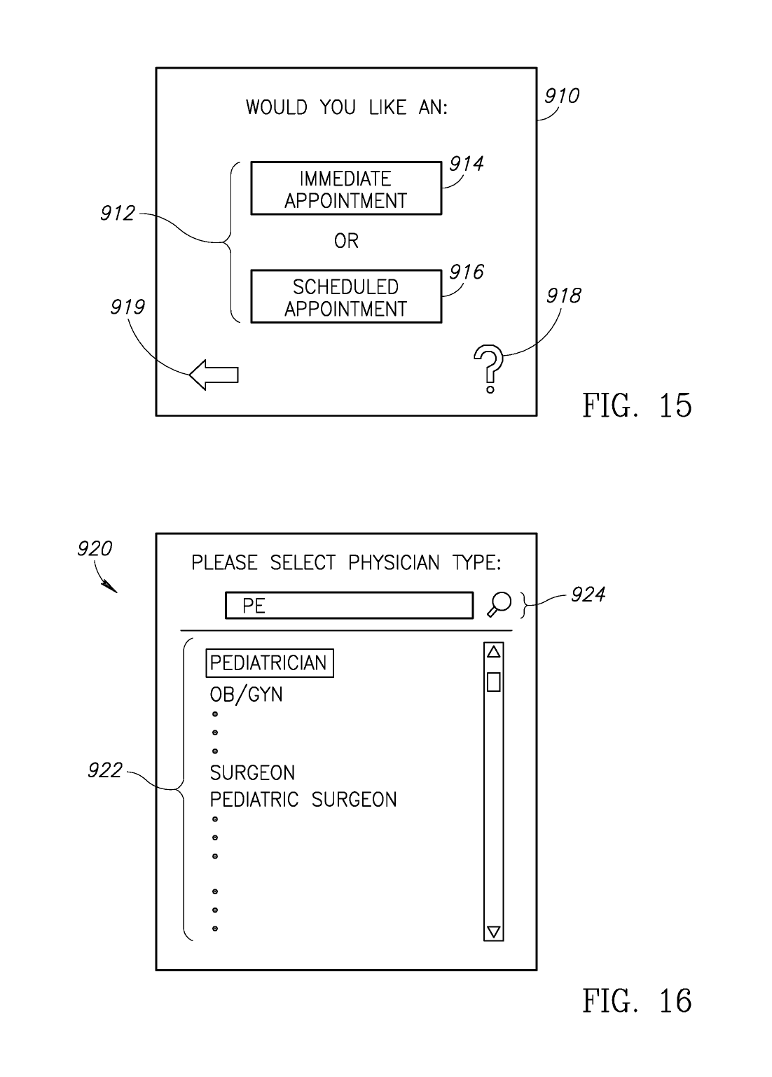

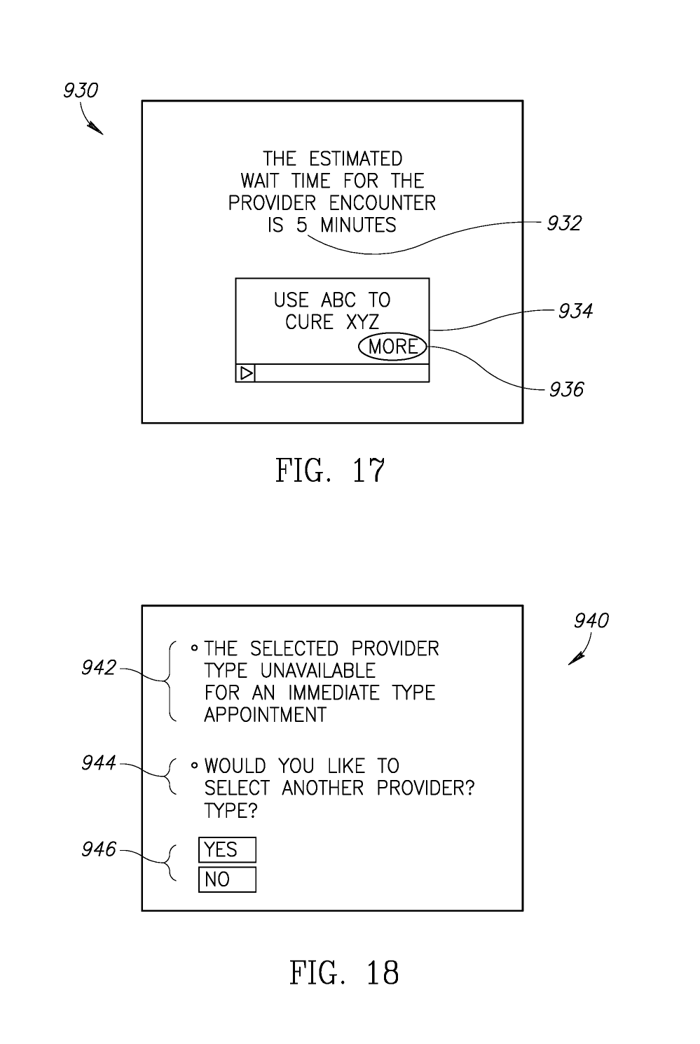

A diagram illustrating a first example mobile device screenshot of estimated wait time for the encounter with the healthcare provider is shown in FIG. 17. In one embodiment, the screenshot of a GUI, generally referenced 930, comprises an estimated wait time (E_{WT}) 932. The GUI is rendered on a rendering device such as computing device of a user. The E_{WT} is calculated by the system and updated in real time. The GUI may also comprise advertisement information generated by or obtained by the system such as information related to an advertisement which may be directed to the patient. For example, knowing that the patient is a diabetic (e.g., determined through analysis of the PAI), the system displays an advertisement (e.g., advertisement information) for diabetes treatment and/or medication from a third party. The duration of the advertisement is determined so that the advertisement fits within a time period (e.g., one minute) of the wait notification. For example, the GUI can be updated in real time to include the updated E_{WT} and a directed message 934 (e.g., “Use abc product to cure xyz condition,” “Use X123 face cream to rejuvenate face,” etc.) which may include still, audio, and/or video content. The system at this time can further provide a user with a selection item 936 for more information about the product or service advertised 934. For example, if the directed message is a medication, the system provides the selection item 936 for the user to receive information about and/or obtain a free sample of, the directed medication.

A diagram illustrating a first example mobile device screenshot indicating the selected healthcare provider type is not available is shown in FIG. 18. In one embodiment, a screenshot of a GUI, generally referenced 940, comprises text indicating that the desired provider specialty type is unavailable for an immediate type appointment 942; an option to select another provider type 944; and buttons for responding either “yes” or “no” by the patient or a CTM 946.

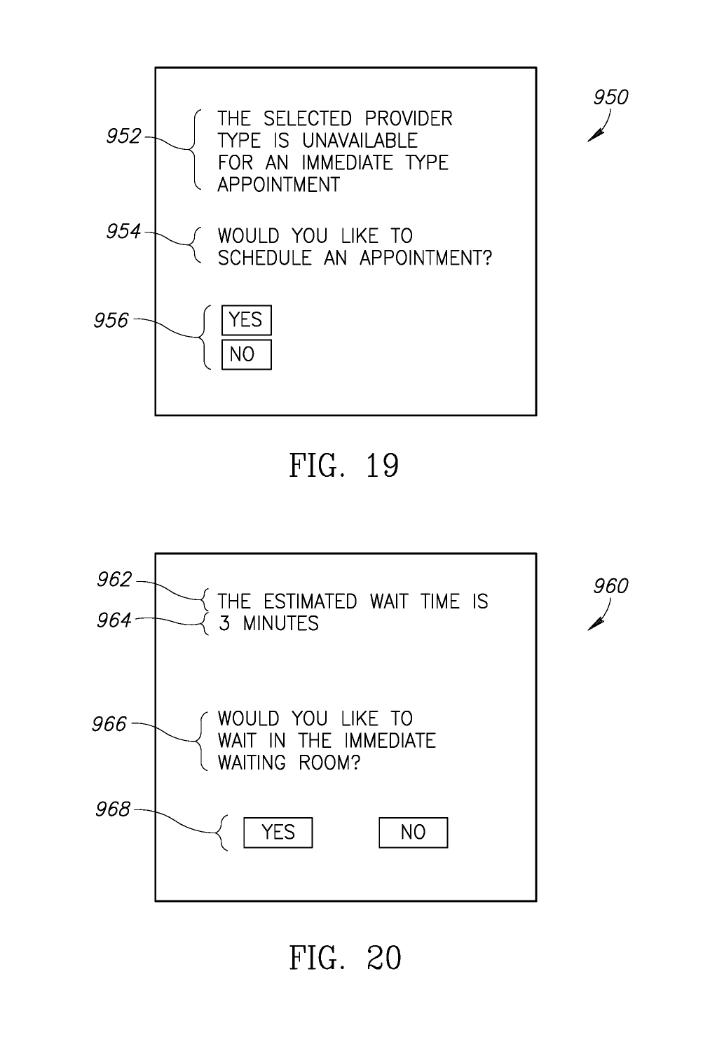

A diagram illustrating a second example mobile device screenshot indicating the selected healthcare provider type is not available is shown in FIG. 19. In one embodiment, a screenshot of a GUI, generally referenced 950, comprises text indicating that the selected physician specialty type is unavailable for an immediate appointment 952; an option to make a scheduled appointment (STA) 954; and buttons for responding either “yes” or “no” by the patient or CTM 956.

A diagram illustrating a second example mobile device screenshot of estimated wait time for the encounter with the healthcare provider is shown in FIG. 20. In one embodiment, a screenshot of a GUI, generally referenced 960, comprises text 962 informing the patient that the estimated wait time is ‘X’ minutes 964 (e.g., estimated wait time (E_{WT})); an option to wait in the immediate waiting room 966; and buttons for responding either “yes” or “no” 968. Note that the estimated wait time (E_{WT}) may be updated in real time.

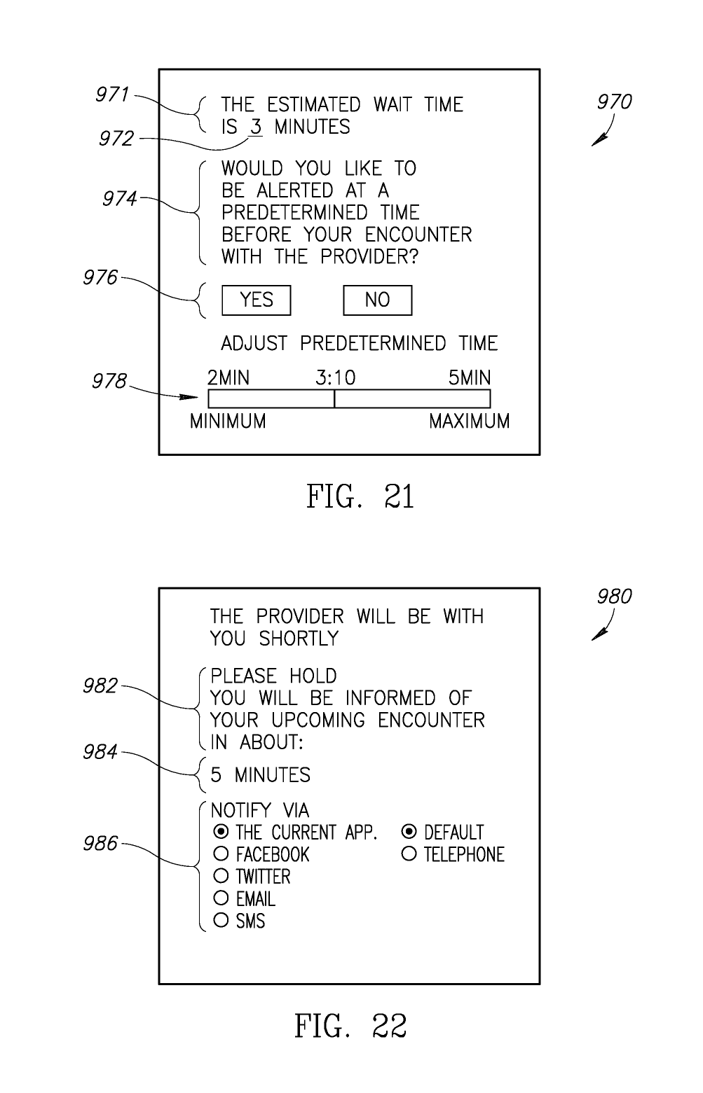

A diagram illustrating a third example mobile device screenshot of estimated wait time for the encounter with the healthcare provider is shown in FIG. 21. In one embodiment, a screenshot of a GUI, generally referenced 970, comprises text 971 informing the patient that the estimated wait time is ‘X’ minutes 972 (e.g., estimated wait time (E_{WT})); an option to be alerted a predetermined time (PT_{W}) before their encounter with the provider 974; buttons for responding either “yes” or “no” 976; and a slider for the user to adjust the predetermined time (PT_{W}) time to a desired value between minimum and maximum values 978. Note that the estimated wait time (E_{WT}) may be updated in real time.

A diagram illustrating an example mobile device screenshot of a reminder for the encounter with the healthcare provider is shown in FIG. 22. In one embodiment, a screenshot of a GUI, generally referenced 980, comprises a notification 982 informing the patient of a wait time (X_{WT}) 984 using any suitable language such as: “The doctor will be with you shortly. You will be informed of your upcoming session in about “X_{WT}” minutes.” Where X_{WT} is the estimated wait time (E_{WT}) less the predetermined time (PT_{W}); X_{WT}=E_{WT}−PT_{W}.

The GUI also comprises selection items 986 to select one or more notification methods, e.g., email, telephone (e.g., a voice call), simple message service (SMS), social media such as Facebook™, Twitter™, etc., and a default notification method. An option may be provided to change the predetermined time (PT_{W}) with the estimated wait time (E_{WT}) updated accordingly. The PT_{W} may be set to a default value or may be based upon a system wait time.

The notification mechanism may be set to a default (e.g., SMS message, voice call, etc.) or may be set in accordance with settings stored in the PAI (e.g., alert by SMS and email), etc.

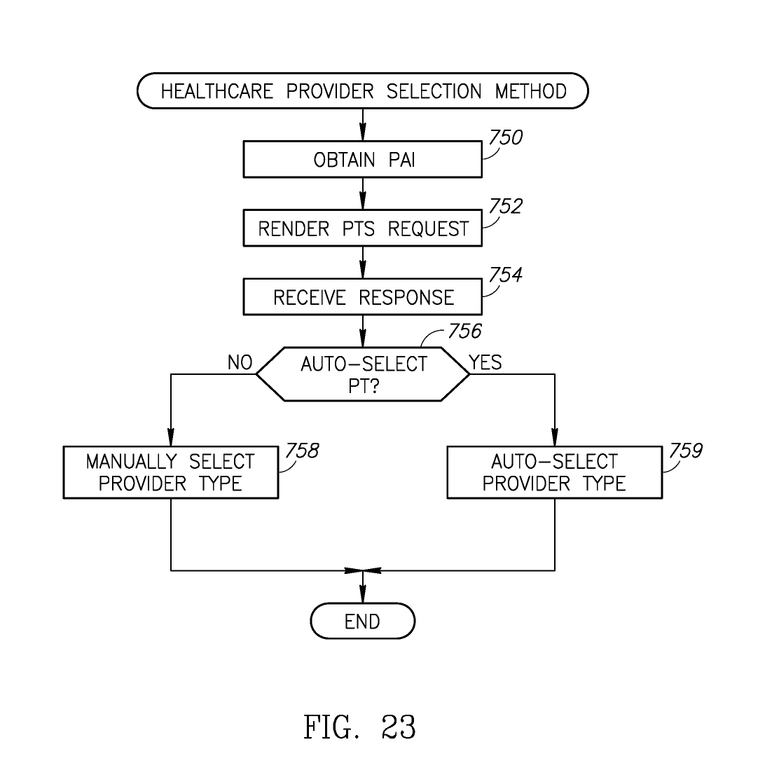

A flow diagram illustrating an example healthcare provider selection method is shown in FIG. 23. First, the patient information (PAI) related to the corresponding patient is obtained (step 750). The PAI is obtained from memory and may include one or more of: user identification information (e.g., 67 year old male, date of birth 24 Dec. 1948, etc.), previous medical history (e.g., diabetic, allergies, etc.), previous medical charts, biometric data (6′5″ tall, 250 lbs., blue eyes, blood type O, fingerprint information, gender, etc.), medication history (e.g., past and current medications prescription and non-prescription), dosage, insurance carrier information (e.g., Medicare™, Oxford™ supplemental, etc.), insurance account information (e.g., account no. 123456789, effective date, expiration date, etc.), credit card information, desired system settings, etc.

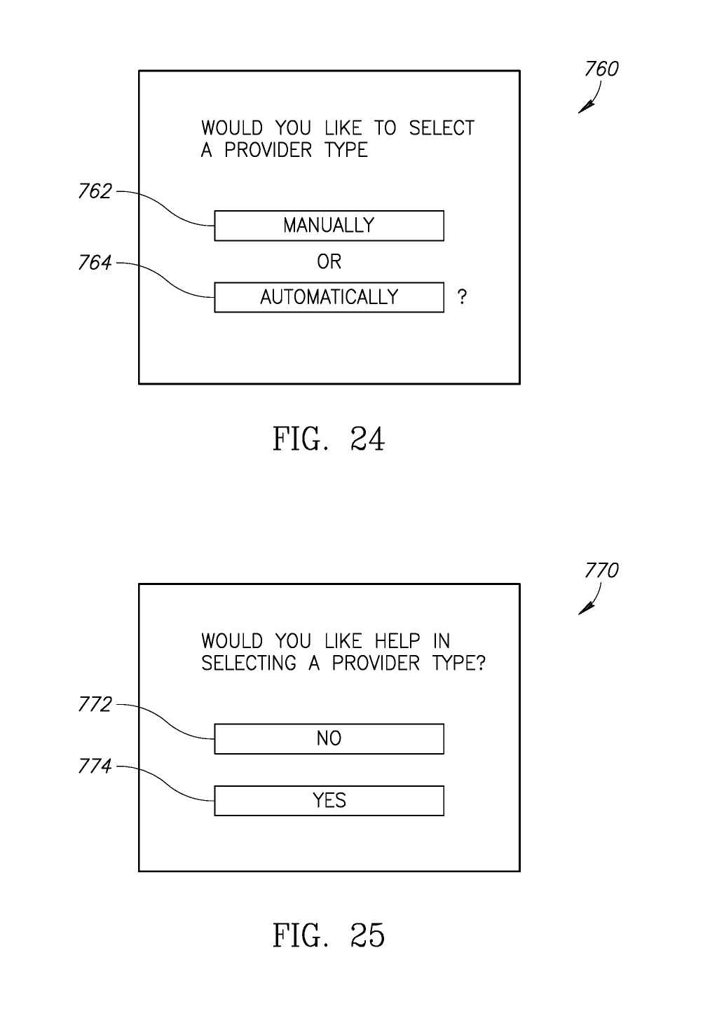

A physician specialty type-selection (PTS) request is then rendered on a computing device (step 752). For example, the PTS may be rendered on the display and/or speaker of the computing device shown in FIG. 24. The GUI 760 comprises an option to select a physician type manually 762 or automatically (e.g., by the system) 764.

The system waits until a response is received from the patient (step 754). The response may be received via any suitable user input device such as via a display (e.g., a touchscreen) or microphone. If the patient selects manual selection (step 756), the patient manually chooses a provider specialty type (step 758). If the patient selects automatic selection (step 756), the system executes an automatic type selection process (step 759), described in more detail infra.

A diagram illustrating an example mobile device screenshot of healthcare provider specialty type selection is shown in FIG. 24. The PTS screen shot 760 is rendered on a computing device of the patient. The PTS request comprises one or more menu items such as “Manual” selection item 762 and “Automatic” selection item 764 for selection by the patient to request physician specialty type manually or automatically, respectively.

A diagram illustrating an example mobile device screenshot of requesting help in choosing a healthcare provider specialty type is shown in FIG. 25. The GUI 770 prompts the patient whether they would like help in selecting a healthcare provider specialty type. displays asks The GUI also comprises one or more menu items such as “no” selection item 772 and “yes” selection item 774 for selection by the patient. In this embodiment, the patient provides information regarding a current medical issue (CMI) for which the patient seeks treatment to permit a recommendation of provider to be generated.

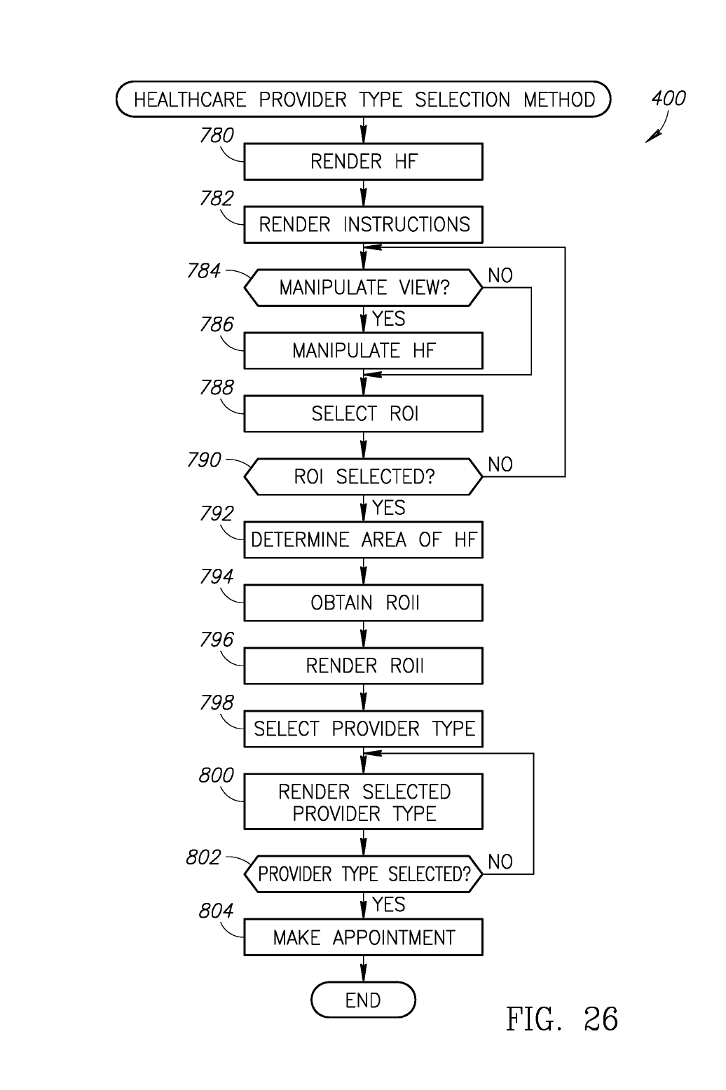

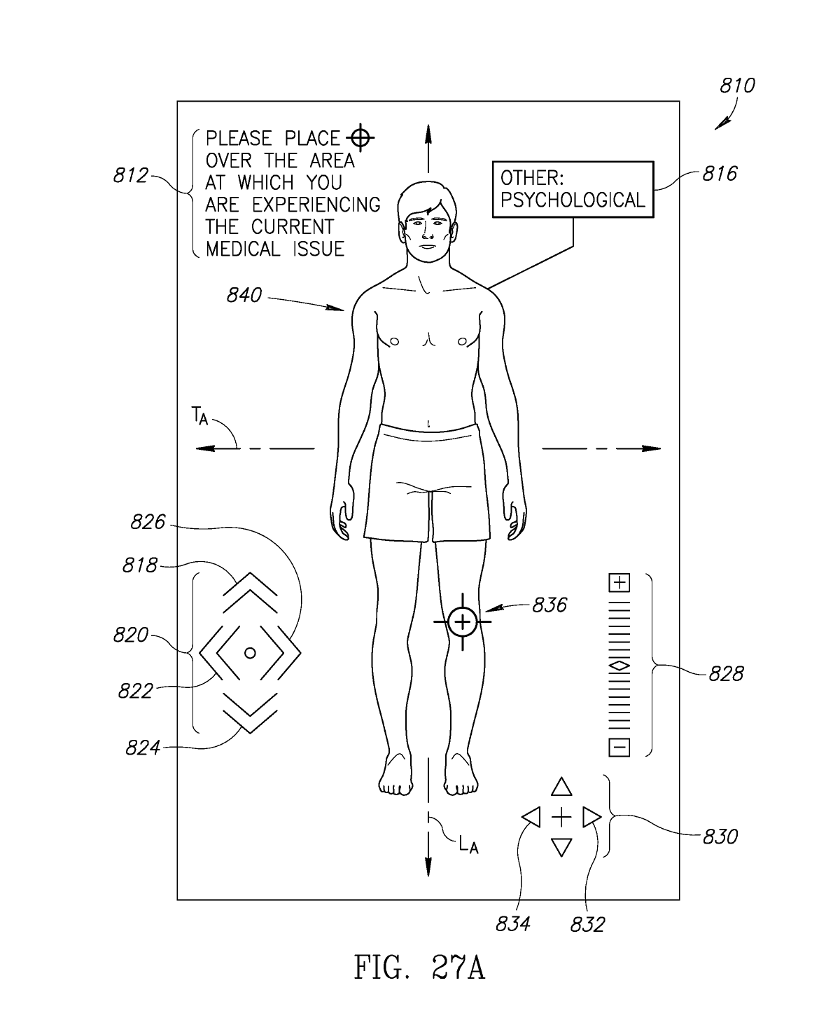

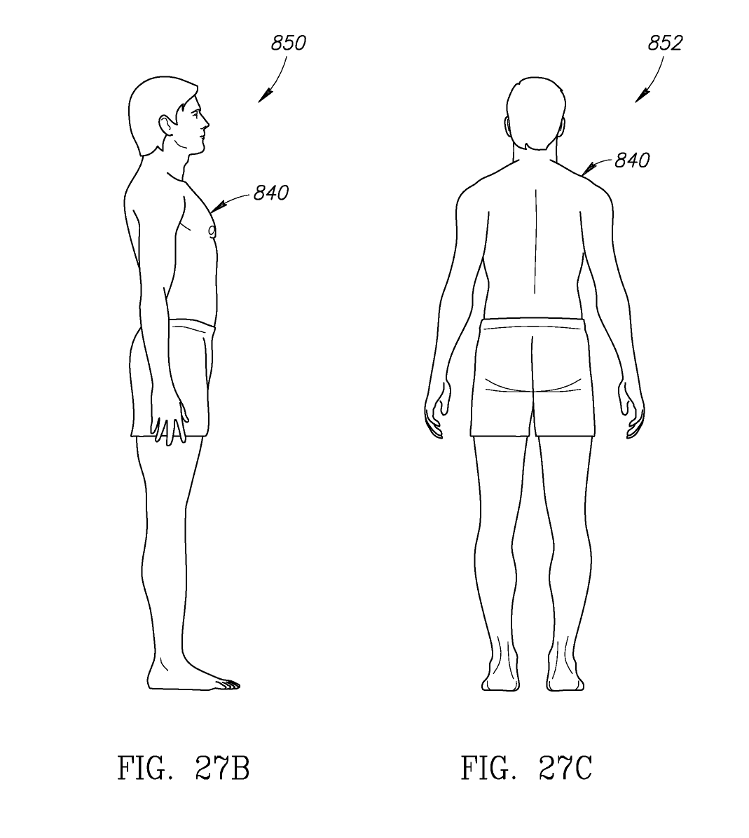

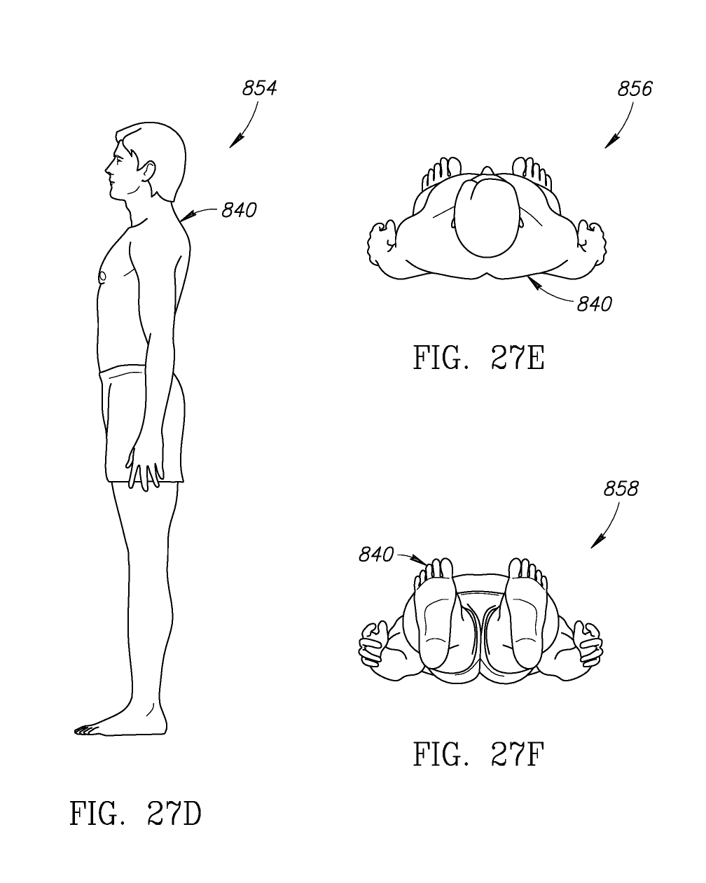

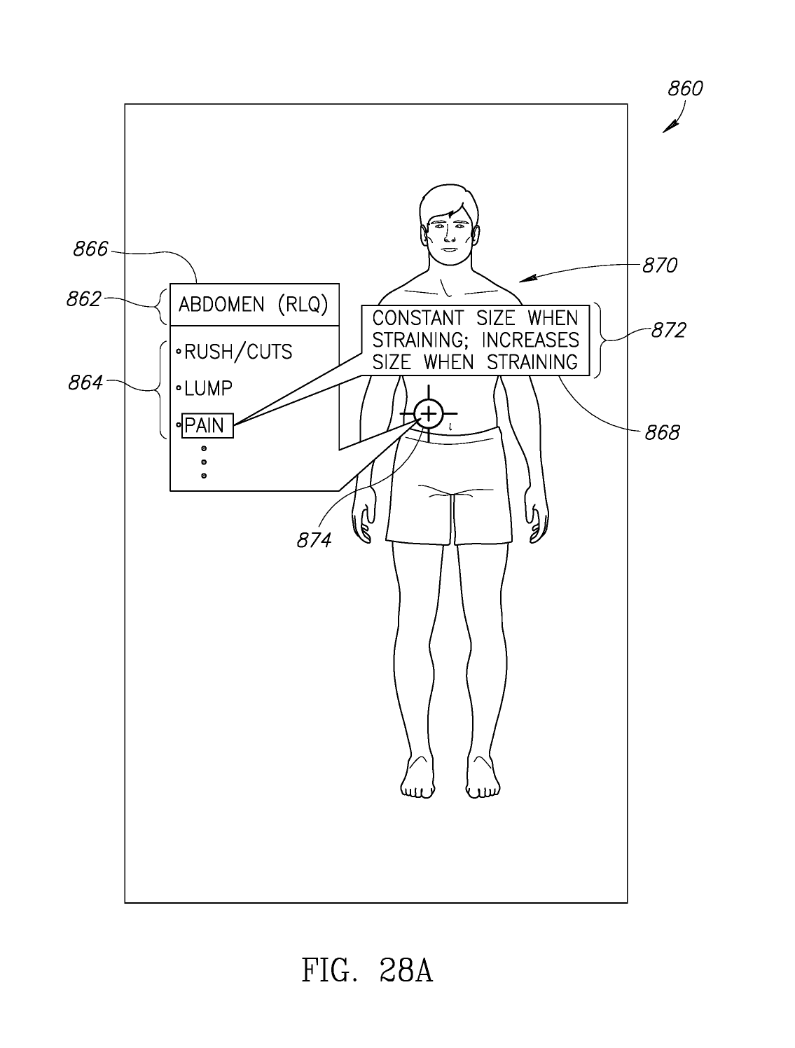

A flow diagram illustrating an example healthcare provider specialty type selection method is shown in FIG. 26. First, a human form (HF) is generated and rendered on the computing device of the patient such as shown in FIG. 27A. The HF may be generated as a 2D or 3D human form and is shown as a 2D HF for clarity sake. It is also assumed that the HF may be represented as a human male or female form as may be selected from memory. For example, a user may initially set a desired shape and/or color of the HF to customize the experience in accordance with patient preferences. In one embodiment, the HF represents the actual anatomy and/or gender of the patient. The system obtains the PAI of the patient and determines their gender from the PAI.

For example, the PAI of the patient is obtained that indicates the patient is female and lost her left leg below the knee due to complications from diabetes. Thereafter, the HF is configured to represent a female without a left leg below the knee or with the left leg being de-highlighted below the left knee to more closely reflect the anatomy of the patient. The HF may be generated using one or more actual images of the patient obtained in real time and/or from memory (e.g., PAI).

Another selection area is displayed whereby the patient can search for a description of a current medical issue (CMI) and/or may enter one or more search terms. This may be used to select CMIs which are difficult to depict graphically and/or select such as psychological issues, depression, etc. Thus, whether to display the HF is optional for the patient. If no HF is to be rendered, a selection area with a list of medical issues and/or corresponding physician types is displayed for selection by a user.

Instructions are rendered (step 782) to permit the patient to select at least one location on the HF which corresponds with areas in which the patient is experiencing the current medical issue (CMI) and for which the patient is seeking treatment (e.g., during an encounter with an HCP). This at least one location may correspond with the ROI. These instructions are referred to as request instructions (RIs).

The RIs may be set/reset by the system and/or the user and may be stored in memory for later use. The RI may be obtained from memory and may be generated in accordance with the PAI. For example, if the PAI indicates that the patient prefers a certain language, e.g., Spanish, then the request is provided in Spanish.

It is then determined whether the patient wants to manipulate a view of the HF (step 784). If so, the view of the HF is manipulated in accordance with the patients input (step 786). Otherwise, the method continues with step 788. The system determines that the patient wants to manipulate the HF when any manipulation command for changing the view of the HF is detected. Manipulation commands may include, for example, pan, tilt, rotate, and/or zoom (in/out) commands. The manipulation commands may be sensed using the user interface (UI) (e.g., keyboards (hard or soft), touchscreen sensors, accelerometers, gyroscopes, orientation sensors, etc., mice, stylus, touchpads, etc.) of the computing device.

Thus, the HF may be manipulated so that a desired area of the HF is displayed and an ROI selected (step 788). At least one ROI is selected at which the patient is experiencing the current medical issue (CMI). The ROI may be selected using the built in UI features of the computing device (e.g., touch, click, etc.). Once selected, the patient can move or delete the selected ROI and select a new one. Note that a plurality of ROIs may be selected.

The ROIs may correspond with the gender of the patient. Thus, the ROI corresponds with the male anatomy for male patients and corresponds with the female anatomy for female patients. Analysis of the PAI can be used to automatically determine the patient's gender. The ROI is then configured to correspond with a patient's gender. The ROI may also correspond with other information in the PAI such as current medical history of the patient (e.g., diabetes, heart condition, etc.), age, etc. For example, if the patient is under a threshold age (e.g., 18, etc.), the ROI may be different from the ROI for a person above the threshold age. In other words, the system may select and render baby, juvenile, young adult, middle aged, and older adult HFs depending upon an age of the patient.

Several examples of selecting ROIs will now be described. If the patient is having abdominal pain in the right lower quadrant (e.g., pain in the right lower quadrant (RLQ) is area at which the patient experiences the CMI), the RLQ of the abdomen of the HF may be selected as a ROI. Similarly, if the patient is having knee pain, the corresponding knee may be selected as a ROI. In a similar fashion, if the patient is having back pain in the left lower quadrant of the back, then this area may be selected as a ROI.

Once the patient has selected one or more ROIs (step 790), an area of the HF associated with the selected at least one ROI is determined (step 792). Note that it can be determined that the patient has selected at least one ROI when an ROI remains stationary for a threshold period of time, e.g., four seconds.

The area may have a corresponding coordinate or coordinates or boundary relative to coordinates of the HF. For clarity sake, it is assumed that the area of the HF associated with the selected ROI defines an anatomical region of a human such as a shoulder, a foot, a right lower quarter abdomen, etc. which has a well-defined boundary.

For example, the HF may be divided into corresponding anatomic regions of a human using any well-known mapping technique such as a 2D or 3D coordinate mapping method, etc. These regions are divided into sub-regions and may be layered. For example, the head may be divided into a right or left temple, a right or left ear, a right or left inner ear, lips, an upper or lower jaw, a chin, left or right eyes, behind left or right eyes, individual teeth, tonsils, throat, left or right inner cheek, mucosa of the mouth, etc. Similarly, a limb such as a right foot may be divided into muscles, skin, bones, such as a tibia, a fibula, femur, joints such as the hip, knee, or ankle, digits such as toes and joints thereof, etc. With regard to layering, if a user selects the abdomen, the upper layer may be skin while lower (i.e. inner) layers) may be mapped to organs within the corresponding area and/or layer of the abdomen, etc.

Each of the regions may be subdivided into sub-regions. One or more of the regions and/or sub-regions may superpose or otherwise overlap other regions or sub-regions. The area of the HF associated with the selected ROI may be scaled in accordance with a scale of the HF. Further, the regions and/or sub-regions may be scaled relative to the HF. For clarity sake, each region or sub-region is referred to by its anatomical name rather than by coordinates as described infra.

Once the area of the ROI is determined, ROI information (ROII) corresponding to the area of the HF associated with the selected ROI is obtained (step 794). The area may be referred to using a corresponding anatomical name such as a shoulder, a chest, a head, a right or left leg, abdomen, etc. or using absolute coordinates of the HF. It is understood that the ROII may be mapped to the corresponding areas of the HF using any suitable method such as by regions and/or by absolute coordinates (e.g., in 2D or 3D) and this mapping (e.g., which may be known as ROII mapping) is stored in memory for further analysis.

As an example, Table 3 below illustrates exemplary ROII for areas of a genderless adult HF using anatomical areas. Note that ROII may be specific for gender, age, medical history (e.g., diabetes, heart condition, crones, Lyme disease, etc.), geographic region, etc. Thus, the ROII may be selected based, at least in part, upon PAI. ROII may be tailored to patients. For example, ROII may be specific to gender (e.g., there may be an ROII table for males which may be different from ROII tables for females), age (e.g., ROII tables for adults may differ from ROII tables for children, and infants), medical condition (e.g., ROII for diabetics may differ from ROII for non-diabetics, etc.), etc. In one embodiment, a learning engine is used to learn ROII based upon historical patient evaluations and/or diagnosis.

TABLE 3
ROI Information (ROII) Table (Genderless, Adult)

| | ROII (selections) | | | | | | |
|---|---|---|---|---|---|---|---|
| | Main | Sub-Selections | | | | | |
| Area | Selection | | . . . | Condition(s) | Physician Type | | |
| of HF | Selections 1 | | Selections M | Medical | Primary | | Tertiary |
| (selected) | (highest order) | Selections 2 | (lowest order) | Issue(s) | (highest order) | Secondary | (lowest order) |
| Abdomen | Rash/Cuts | | . . . | Dermal | GP | Internist | Dermatologist |
| (right | (external) | | | | | | |
| lower | Lump | Constant Size | . . . | Tumor | Internist | Gastroenterologist | GP |
| quarter | | when | | | | | |
| (RLQ)) | | straining | | | | | |
| | | Increases in | . . . | Hernia | GP | Hernia | Surgeon |
| | | size when | | | | Specialist | |
| | | straining | | | | | |
| | Pain | Constant/ | . . . | N/A | Internist | Surgeon | Gastroenterologist |
| | | Intermittent | | | | | |
| | | Increases | . . . | Sprain/ | Hernia | Surgeon | Gastroenterologist |
| | | when | | Hernia | Specialist | | |
| | | coughing, | | | | | |
| | | lifting, or | | | | | |
| | | standing | | | | | |
| | Bloating | | . . . | . . . | GP | Internist | Gastroenterologist |
| Eye(s) | All | | . . . | | Optometrist | Internist | |
| Knee | Pain | | . . . | | Orthopedist | Internist | |
| | Swelling | | . . . | | Orthopedist | Internist | |
| | Rash/Cuts | | . . . | | Internist | Dermatologist | |
| | Noise | | . . . | | Orthopedist | Internist | |
| Shoulder | Pain | | . . . | | Internist | Orthopedist | |
| | Swelling | | . . . | | Internist | Orthopedist | |
| | Rash/Cuts | | . . . | | Internist | Orthopedist | |
| | Other | | . . . | | Internist | Orthopedist | |
| Skin | Simple | | . . . | | GP | Dermatologist | |
| | Cuts/Bruises | | | | | | |
| | Redness, | | . . . | | Dermatologist | Internist | |
| | Rash, Itch and | | | | | | |
| | all other skin | | | | | | |
| | conditions | | | | | | |
| Neck/Throat | Pain/Infection | | . . . | | Internist | GP | Ear-Nose- |
| | | | | | | | Throat |
| | | | | | | | (ENT) |
| | | | | | | | specialist |
| | Lump | | . . . | | GP | Surgeon | |
| Chest | Cold | | . . . | | Internist | Cardiologist | |
| | Pain/Shortness | | . . . | | Cardiologist | Internist | |
| | of Breath | | | | | | |
| . . . | . . . | . . . | . . . | . . . | . . . | . . . | . . . |
| Other | Fainting | | . . . | | Internist | Internist | GP |
| | Fear/Anxiety | | | | Psychologist | Internist | GP |
| | Depression | | . . . | | Psychologist | Internist | GP |

As indicated in Table 3, each anatomical area of the HF has corresponding ROII selections associated with it. The ROII selections include one or more main selections (e.g., Selection 1, the highest order selection) and one or more corresponding sub-selections (e.g., Selections 2 through M, where M is an integer) the latter of which may be referred to as dependent selections and may be dependent upon a previous selection in order of dependency (e.g., Selection 2 has a higher order of dependency than Selection M). In other words, the selections are ordered by dependency. Thus, for example, the Selection 2 may be dependent upon Selection 1, Selection 3 may be dependent upon Selection 2, and Selection M may be dependent upon Selection M-1, etc.

Thus, an area of the HF corresponding to a selected ROI has a corresponding ROII as set forth by the selections (e.g., Selections 1 through Selections M (generally Selections-x)). When an ROI is selected, a corresponding anatomical area (e.g., RLQ, etc.) and associated ROII selections as may be set forth in Table 3 above are obtained.

The ROII can be modified by the system and/or user and stored in memory for later use. The ROII may correspond with gender, age, medical history, etc., of a patient. For example, all or a portion of the ROII may correspond with gender, age, medical history, etc., of a patient. Thus, ROII for females may be different than ROII for males. The system determines the gender of the patient through an analysis of the PAI and obtains ROII corresponding to the gender of the patient. For example, for an abdominal ROI, ROII for a female includes information corresponding to female only issues such as pregnancy and female reproductive organs and/or other female issues; while ROII for this same area for males includes information corresponding to male only issues and/or male reproductive organs.

If it is determined during step 788 that a right lower quarter (RLQ) of the abdomen was selected as the ROI, the system obtains corresponding ROII such as (e.g., “rash/cuts (external),” “lump,” “pain”). Similarly, if it is determined that the throat was selected as the ROI, the system obtains corresponding ROII (e.g., “pain/infection,” “lump”). The ROII is placed according to the order of dependency which corresponds with the highest (e.g., selection 1) to lowest order (e.g., selection M). Thus, the lowest order ROII may be a subgroup of the higher order ROII. In other words, the ROII may be grouped and/or sub-grouped.

Thus, a medical condition can be estimated based upon the ROI and/or the ROII selections. The estimated condition is then used to select provider specialty types, and/or for estimating wait times.

The ROII corresponding to the selected ROI(s) is then rendered using any suitable method such as rendering the ROII as selection items which may be located within text boxes, menu boxes, and/or the like (step 796). Associated ROIIs for any additional ROI selections by the patient are then determined. This process is repeated for each level of ROII from the highest order to the lowest. For each of the selected higher order ROIL corresponding lower order ROII are obtained and rendered.

Highest order ROII are rendered using any suitable format such as selection in a selection area. Area information is also rendered that identifies an area of the HF (e.g., see Table 3) that corresponds with the ROI such as “Abdomen (RLQ).”

After all, or a threshold number of levels (as may be set by the system and/or user) of ROII are obtained, a provider specialty type is selected (step 798). It is determined whether a sufficient number of levels of ROII have been selected. For example, although there may be a plurality of levels of dependency for ROII for a current ROI, the process of selecting one or more levels of ROII for the ROI may have been prematurely terminated.

In one embodiment, the system selects at least one provider type based upon an analysis of the selected ROII. The provider type is selected using any suitable method and/or algorithm. For example, the system may employ a table lookup and/or a more complex analysis method such as neural networks, etc. to select the provider type. In one embodiment, at least one alternative recommended provider type is also chosen. This alternative provider type has a lower weight than the primary provider type. For example, assuming that a primary provider type has the highest determined weight, the secondary provider type has a next highest determined weight. A table lookup mechanism may be used whereby provider types are assigned to each selection type and/or sub-selection types within the ROII and may be selected based upon their assignment.

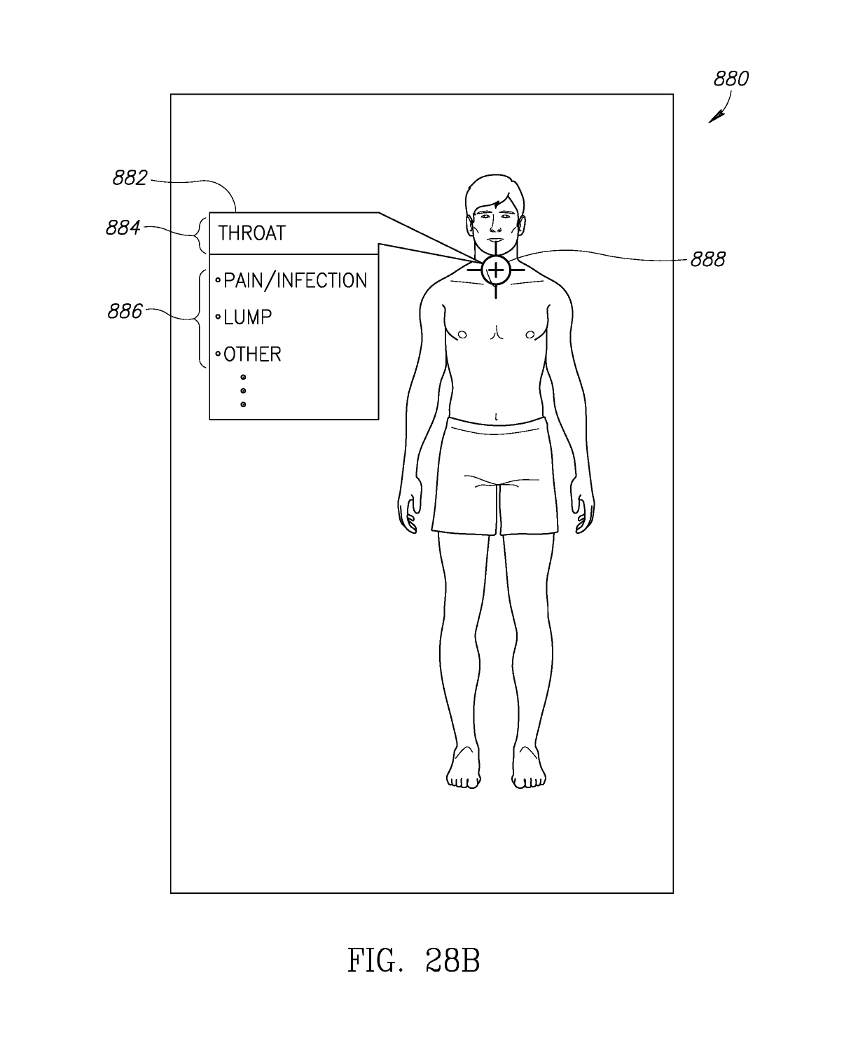

For example, with regard to the ROI 874 (e.g., Abdomen (RLQ)) of FIG. 28A and Table 3), if the patient selected the “Increases size when straining” selection item of the ROIL a general practitioner (GP) is selected as the primary provider type, and a hernia specialist as the secondary provider type and may optionally select a surgeon as the tertiary provider type. If the patient selected the “Constant size when straining” selection item, an internist is selected as the primary provider type, a gastroenterologist as the secondary provider type and a GP as the tertiary provider type.

In a similar manner, with regard to the ROI 888 (e.g., Neck/Throat) of FIG. 28B and Table 3, if the patient selected the “Pain/Infection” selection item of the ROIL an internist is selected as the primary provider type, a GP as the secondary provider type and an ear-nose-throat (ENT) specialist as the tertiary provider type. If the patient selected the “Lump” selection item, a surgeon is selected as the primary provider type.

In one embodiment, the provider types have a weight whereby the primary provider type is the highest weighted provider type and the non-primary provider types have lower weights. If the primary provider type is unavailable (e.g., due to a long wait, not being logged into the system, etc.), a provider from the next highest non-primary provider types is selected. This helps ensure that the patient will be attended to promptly.

During step 798 information associated with the selected provider type is obtained such as, provider availability, wait time, patient ratings, accepted insurance, etc. For example, the wait time reflects an estimated wait time for a patient to start an encounter with a provider of the selected type. For example, if the selected type is an allergist, the wait time for allergists as a group or independently is determined, e.g., an average wait time for providers that are currently logged into the system of the selected type.

In one embodiment, some ROIs may be configured so that when selected, a patient does not need to enter a corresponding ROII selection to select a corresponding provider. For example, with regard to Table 3, when the ROI related to the “Eye” is selected, a provider type is selected by default. Thus, a provider type can be selected without the need for the patient to enter an ROII selection. In one embodiment, a learning application is operative to learn preferred provider types for each ROI a patient has experienced CMI and selects a provider type based upon this historical information. For example, a patient with a certain eye condition is recognized (e.g., by recognizing PAI of the patient). If the patient enters an ROI corresponding to the eye, the system recognizes this and automatically selects a provider type(s) such as an eye specialist that the patient has historically selected for this CMI. This can conserve system resources and/or time.

In one embodiment, the provider type is selected when a request to select a provider type is received from the patient. This request is generated prior to all ROII being selected (e.g., with the currently selected ROI and/or ROII)). Thus, the patient selects a provider type(s) prior to entering all ROII for the corresponding ROI. For example, a physician type(s) is selected only when an ROI is selected and/or when at least one ROII is selected for a corresponding ROI. In other words, even if there is an ROII for a corresponding ROI and the patient has not selected any ROII from this ROII or may have selected less than all orders of ROIL a provider type(s) is chosen based upon the selected ROI and ROII.

For example, with reference to Table 3, assuming that the patient selects the Abdomen (RLQ) as the ROI and selects only the highest order (e.g., the main selection) from the ROII selection such as the “lump” ROII selection. A provider is chosen from providers that can be assigned to all selections relating to the highest order selected ROII such as an internist and/or GP as the primary provider type, a gastroenterologist and/or hernia specialist as the secondary provider type, and a GP and surgeon as the tertiary provider type. In this case, one of each provider type is selected for at least one of the primary through tertiary (or lowest order) or provider types are selected using a predetermined conflict resolution method such using assigned weights for each provider type.

For example, each provider type is weighted, at least in part, with respect to the corresponding ROI and/or ROII. Accordingly, when two or more provider types correspond to a ROI or highest order selected ROII, only the highest weighed provider type is selected for each of the selected two or more provider types. For example, considering the Abdomen (RLQ) ROI, if the Internist has a greater weighting than the GP (for the primary provider types), then the Internist is selected for the primary provider type. This is performed for each order of provider types. The weighting of the provider types may be set by a user and/or the system as well as stored in memory for later use.

In one embodiment, selected (e.g., patient selected) ROII inputs are weighted using any suitable well-known modeling method such as heuristic analysis, neural network analysis and the like to determine the provider type. For example, the patient selects a plurality of ROII selections and an analysis is performed on these selections to determine the provider type.

Information such as provider availability (individually and/or as a group by provider type, state, etc.), waiting time (individually and/or as a group by physician type, state, etc.), patient rating, insurance type, payment type (e.g., insurance type), and/or other suitable information are used as inputs to an algorithm such as a heuristic analysis, etc., to determine the provider type and/or to select providers. This information is stored in memory for later use. Some of the information input into the algorithm is obtained in real time. For example, the heuristic analysis is performed upon the ROII selections and/or other inputs such as provider availability, waiting time, patient rating, payment type, etc. to determine the provider type.

Once the provider type is chosen, the selected provider type is rendered on the computing device of the patient. The provider type is rendered as selection items for the user. At least one of the determined primary, secondary, and tertiary provider types is rendered. Alternatively, a set number of selected provider types (e.g., 1, 2, etc.) chosen by the patient (e.g., in the PAI) are rendered.

Further, an option may be provided to the patient to select another provider type. The patient is provided a list of provider types for manual selection by the patient.

Once the patient selects a provider type (step 802), the method continues with making an appointment (step 804) described in more detail supra in connection with FIGS. 11A, 11B, 11C, 11D, 11E, and 11F. Otherwise, the method returns to step 800.

A diagram illustrating a first example mobile device screenshot showing a human body for conveying location of current patient medical issue is shown in FIG. 27A. A screenshot of a GUI 810 comprises an HF 840 generated as a 2D or 3D human form (2D shown). The patient is instructed 812 to place an indicator over the area where the patient is experiencing the current medical issue. One or more manipulation methods are provided to manipulate the view of the HF (e.g., rotate, pan, tilt, zoom, etc.) about one or more corresponding axes (or planes) such as a longitudinal axis (L_{A}) and/or a transverse axis (T_{A}). For example, a dragging motion to the left or right rotates the HF about the longitudinal axis (L_{A}) and a dragging motion up or down rotates the HF about the transverse axis (T_{A}). The rotational manipulation can be performed at a location off the HF so that the ROI selection can be performed without unintentionally rotating the HF. In addition, the GUI comprises place 816 for the patient to indicate CMIs that do not lend themselves to touching an HF image.

In one embodiment, the rotational manipulation mechanism comprises one or more selection items 818, 822, 824, and 826 which when selected rotate the HF about one or more corresponding axes, e.g., L_{A} and/or T_{A}. The rotation selection items are configured to rotate the HF about the corresponding axis.

The manipulation selection items further comprise a mechanism to zoom in/out, e.g., pinch to zoom. Zoom selection items 828 (e.g., +/−indicators) are provided to allow the user to zoom the view of the HF in or out. Manipulation commands for changing the view of the HF comprise pan, rotate, and/or zoom (in/out) commands and are also provided.

A region-of-interest (ROI) selection item is also provided which can be manipulated to select an ROI of the HF at which the patient is experiencing a current medical issue (CMI). For example, the ROI can be selected by pressing and holding an area which superimposes the HF (e.g., for a predetermined time period such as five seconds, etc.) while the manipulation selection items are selected by manipulating an area of the screen which does not superimpose the HF. An ROI target indicator 836 can be dragged and/or dropped on the HF to indicate the corresponding area of the HF. The user can zoom in/out using the zoom command which is superimposed on the HF at or near the ROI.

Further, the ROI target indicator 836 is moved using any suitable method such as by using selection arrows 830 which moves the ROI target indicator 836. Thus, for example, selecting a right arrow 832 or a left arrow 834 moves the ROI target indicator 836 rightward or leftward, respectively across the HF.

Note that in this example embodiment, the longitudinal axis (L_{A}) is parallel to sagittal and/or coronal planes relative to the HF and the transverse axis (T_{A}) is parallel to a transverse plane of the HF. The invention contemplates other axis orientations as well.

A diagram illustrating a second example mobile device screenshot showing a human body for conveying location of current patient medical issue is shown in FIG. 27B. A screenshot of GUI 850 shows rotation of the HF 840 about its longitudinal axis (L_{A}) by about 90 degrees to show a right side view of the HF.

A diagram illustrating a third example mobile device screenshot showing a human body for conveying location of current patient medical issue is shown in FIG. 27C. A screenshot of GUI 852 comprises the HF 840 rotated about the longitudinal axis (L_{A}) by about 180 degrees to show a rear view of the HF.

A diagram illustrating a fourth example mobile device screenshot showing a human body for conveying location of current patient medical issue is shown in FIG. 27D. A screenshot of GUI 854 comprises HF 840 rotated about the longitudinal axis (L_{A}) by about 370 degrees to show a left side view of the HF.

A diagram illustrating a fifth example mobile device screenshot showing a human body for conveying location of current patient medical issue is shown in FIG. 27E. A screenshot of GUI 856 comprises HF 840 showing rotation of the HF about the transverse axis (T_{A}). The HF may be rotated about the transverse axis (T_{A}) by about 90 degrees to show a top view of the HF.

A diagram illustrating a sixth example mobile device screenshot showing a human body for conveying location of current patient medical issue is shown in FIG. 27F. A screenshot of GUI 858 comprises HF 840 rotated about the transverse axis (T_{A}) by about −90 degrees to show a bottom view of the HF.

A diagram illustrating a first example mobile device screenshot showing a human body with a list of possible medical issues based on the patient's selection is shown in FIG. 28A. A screenshot of GUI 860 comprises graphical representation of a front view of an HF 870 with a plurality of anatomical regions. An ROI 874 (e.g., an abdomen (RLQ)) is selected and corresponding ROI information (ROII) for this ROI is determined. This ROII is then rendered in any suitable form such as using ROII selection items 864 and 872 by order of dependency. Thus, if the patient selects a ROI, the corresponding ROII is obtained and rendered by order of dependency. This process is repeated for each level of ROII from the highest to the lowest order. For example, for each of the selected higher order ROII, corresponding lower order ROII are obtained and rendered. Selection items 864 and 872 are rendered in a menu box such as menu boxes 866 and 868, respectively. The system renders highest order ROII using any suitable format such as selection items 864 in the selection area 866. Area information 862 is also rendered that identifies an area of the HF determined to correspond with the ROI such as “Abdomen (RLQ)” in the present example in the association with the ROII such as the highest-order ROII. For each level of ROII, new selection items are generated and/or renders optionally within a corresponding menu box or menu area.

A diagram illustrating a second example mobile device screenshot showing a human body with a list of possible medical issues based on the patient's selection is shown in FIG. 28B. A screenshot of GUI 880 comprises graphical representation of a front view of an HF with an ROI 888 selected. The ROI is selected and corresponding ROII rendered as selection items 886. In contrast to the example with respect to FIG. 28A, there is only a single level of ROII and no dependent ROII in the current example. Area information 884 is rendered that identifies an area of the HF that corresponds with the ROI such as “Throat” in the present example.

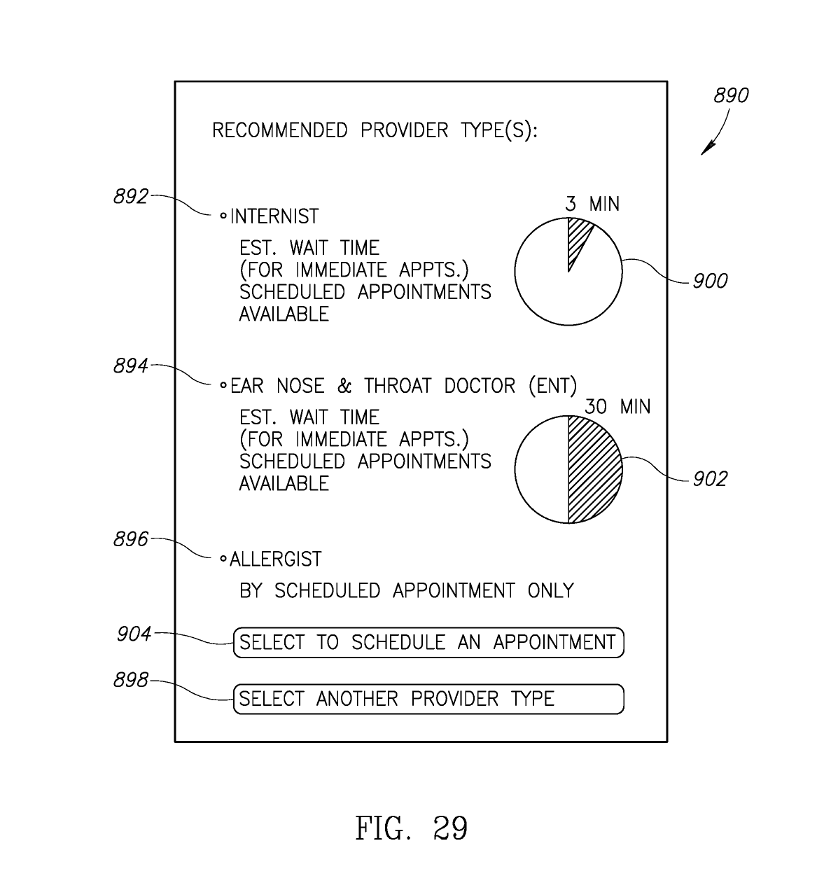

A diagram illustrating an example mobile device screenshot of recommended healthcare provider specialty types in accordance with the patient's selections is shown in FIG. 29. A screenshot of GUI 890 comprises one or more selected provider types 892 through 896. Associated information such as wait times 900 for the one or more selected provider types is rendered in association with the corresponding provider type (e.g., there is wait time as opposed to appointment only and/or the corresponding physician type is currently accepting patients). The system also determines whether a wait time (e.g., for an immediate type appointment) for the corresponding provider type is greater that a predetermined threshold (e.g., 35 minutes, etc.) and/or is otherwise unavailable. If so, an indication of such is shown, i.e. appointment only indication 904 which indicates that the corresponding provider type is only available for scheduled type appointments.

An option can be provided for the patient to select another provider type, i.e. selection item 898 which can be selected by the patient. When selected, the system provides the patient with a list of provider types for manual selection by the patient.

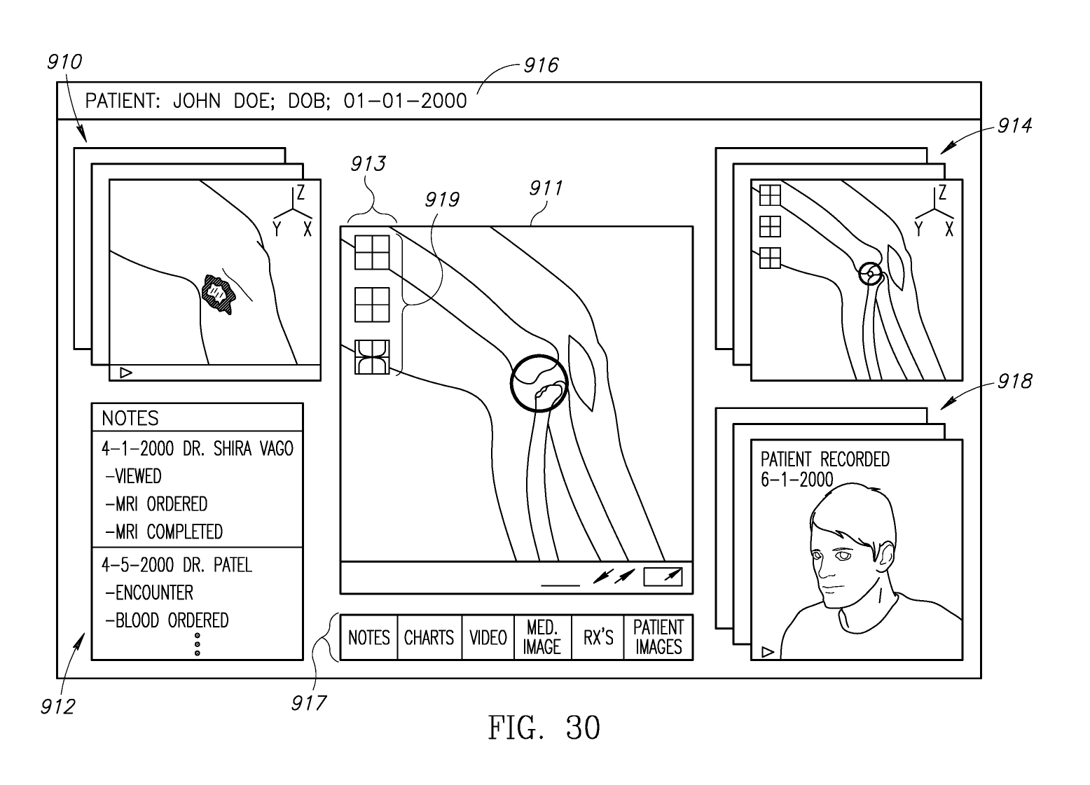

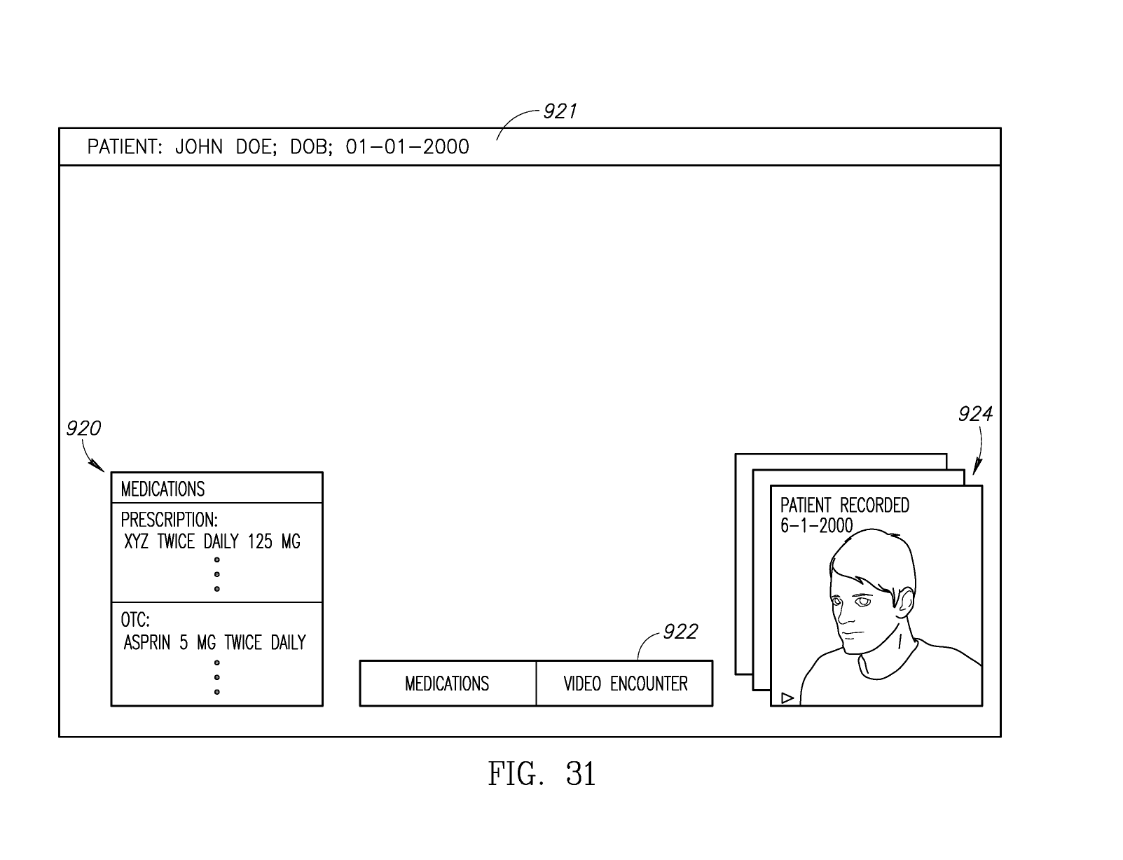

A screen shot of an example advanced radiology GUI generated in accordance with the present invention is shown in FIG. 30. The GUI comprises one or more images 910 generated using image information acquired by one or more cameras of USs of the system and one or more medical images 914 generated using reconstructed medical image information acquired by one or more medical imagers of the system. The GUI also comprises information related to the patient such as medical records, prescriptions, notes, graphs, etc., such as may have been acquired in association with one or more encounters with the patient. Thus, the GUI, or portions thereof, can be generated and displayed during an encounter with a patient and/or at other times such as when information (e.g., test results, medical images, etc.) is obtained. For example, an HCP may order a test such as an ultrasound exam on a patient, and when ultrasound information is transmitted to the system it is stored in memory and used to populate a corresponding section of the GUI such as medical images 914. One or more of medical images 914 may be selected and enlarged such as shown by medical image 911. The medical images include location bars 919 so that a viewer such as the HCP may determine a plane and location.

The GUI also comprises video information 918 such as information obtained before and/or during an encounter with a provider (e.g., HCPs, HCWs, CTMs, and/or the patient). Images and videos may be offset and stacked so that a viewer can more easily select desired images and/or videos. All information acquired by the system with regard to the patient such as information obtained during an encounter may be recorded.

For example, a glucose sensor acquires blood sugar readings and forwards this information to the system and used to generate a corresponding chart and display.

Further, the GUI comprises a menu bar 917 having a plurality of selections: view, notes, charts, video, medical images, prescriptions (Rx), and patient images, or other data. A user may close windows as desired.

Information entered by a user is displayed in corresponding groups. For example, notes entered by users of the system with regard to the patient may be stored and viewed in a notes window 912. Access to information in the GUI is typically restricted in accordance with the access rules configured for the patient. If a CTM is assigned to the patient subject to the User A entries (see Table 1), the CTM is limited to viewing video conference and medications only.

A user such as the HCP or a HCW may interact with the images 910 and the medical images 914. In particular, the enhanced radiology block 713 (FIG. 5) registers images and thereafter superimposes or links them. For example, images 910 and medical images 914 may be registered and/or superimposed and/or otherwise linked to each other. Further, the registered images can be linked and superimposed with each other using any suitable layering scheme so that a user, e.g., HCP, can view them and/or switch between them in rendering a diagnosis.

An advanced radiology GUI generated in accordance with the present invention is shown in FIG. 31. This GUI is generated and/or rendered when the CTM attempts to view the advanced radiology GUI of FIG. 30. In this example, however, the CTM is not authorized to view the information included in the GUI of FIG. 30 but rather only video conference information and prescription information. Accordingly, the system renders only the allowed content such as the video conference information 924 and medication information 920 in corresponding windows. A menu bar 922 is generated in accordance with the privileges configured for the CTM.

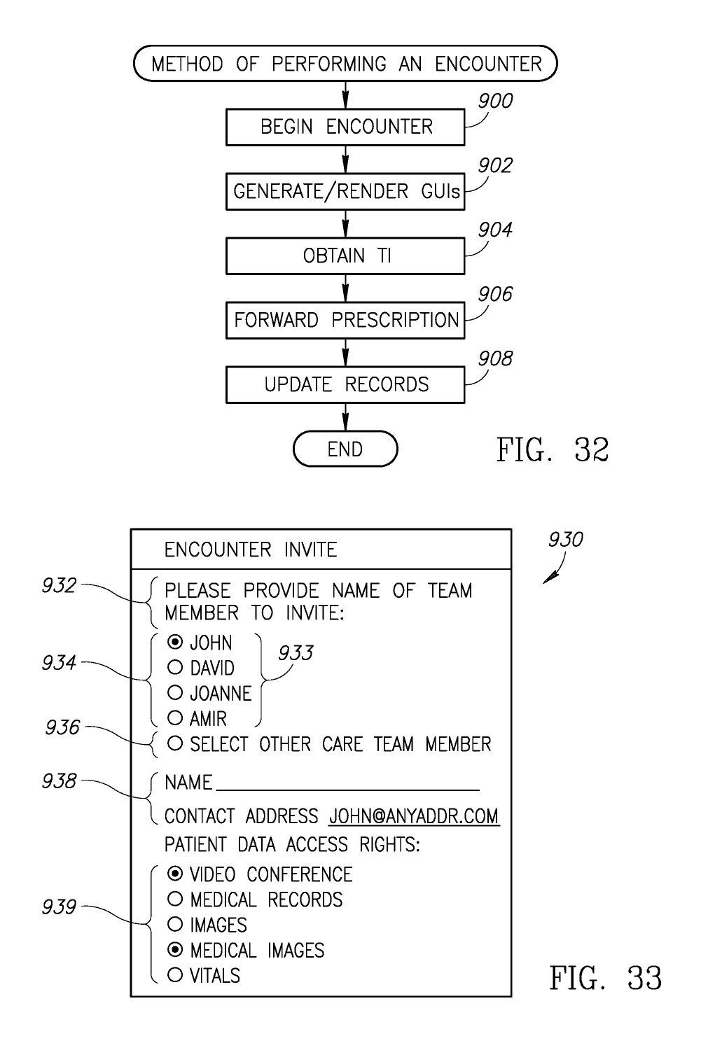

A flow diagram for performing an encounter in accordance with the present invention is shown in FIG. 32. The system begins an encounter (e.g., video) between an HCP and a patient. Encounters are typically launched by providers when they are ready to see a patient. Alternatively, encounters can be initiated by a user such as an HCW visiting the patient, a patient, and/or a CTM. With regard to in field HCWs, these HCWs may be selected by a HCP to visit the patient or may be currently visiting the patient. For example, when an HCW and/or a worker at a facility in which the patient is located such as a nursing home, a convalescence home, and the like, is currently visiting the patient, the HCW can initiate an encounter between the patient and the HCP. When an HCW determines that the patient requires that his/her needs be attended to by an HCP, the HCW requests an encounter between the HCP and the patient.

To launch the encounter, the system establishes a communication channel such as a video channel to transmit and/or receive video information between two or more parties such as one or more of a HCP and the patient, a HCW, and/or a CTM. For example, the system establishes a bidirectional video communication channel between the patient and/or CTM, HCW and HCP. For example, when an HCW is visiting the current patient, the current patient and the HCW may communicate to a HCP such as a provider located remotely from the patient.

Note that patient may transmit an encounter invitation to one or more selected CTMs which may include a link to join the encounter between the patient and the HCP. This is useful when a CTM provides care to the patient. The CTM may have been previously selected by the patient or may be selected on a need basis (e.g., by invitation) and may have patient data access rights assigned thereto. For example, the system generates a GUI providing the patient with options for contacting CTMs on a need basis. The GUI includes options for selecting patient data access rights to be assigned to the CTM for the current encounter. All parties to an encounter may join and/or leave (i.e. log off) an encounter at will.

The system generates and renders one or more GUIs on the US of the parties to the encounter (step 902). These GUIs comprise video information for one or more of the parties of the encounter. For example, one or more of the parties to the encounter can view a live video feed of other parties to the encounter.

The system comprises view setting information (VWI) which may be set by the system and/or user in accordance with the privileges and authorization configured for the user. The VWI sets the look and feel of an interface rendered during an encounter and is set by a user and/or system. For example, a first HCP such as an electrophysiologist (EPS) may prefer to view medical charts such as an ECG while a radiologist may prefer to view medical images of a patient. Accordingly, the EPS sets system settings such that the system renders medical charts (e.g., ECGs) when conducting an encounter rather than images unless specifically selected. This enables the EPS to view medical charts and the radiologist to view medical images as a default setting during a video encounter. Accordingly, the system can employ a learning function which learns a user's preferred settings for the VWI over time. Note that in all cases, the information available for display is in accordance with the privileges and authorization configured for each user.

Treatment information (TI) is then obtained from the HCP (step 904). The TI comprises information entered by the HCP with respect to the encounter such as notes, a recommended course of action with regard to treatment of the patient, treatment instructions for those assigned to the patient such as HCWs and/or CTMs, prescriptions, work ordered (e.g., drug test), charts, annotations to information, etc. for the current encounter with the patient.

Any prescriptions written by the HCP are forwarded to the pharmacy selected by the patient (step 906). This may be a default patient selected pharmacy or one that is closest to the patient. The pharmacy may arrange for the prescription to be delivered to the patient and/or the patient or a representative thereof such as a CTM may pick up the prescription. The pharmacy provides the system with one or more updates of the status of a prescription. Pharmacies typically employ identification methods to identify an authorized party to pick up a prescription. For example, the pharmacy requires a threshold number (e.g., three, etc.) of forms of identification such as a name, a fingerprint, and facial identification.

Instructions for care are forwarded to the patient and/or the corresponding HCW(s) and/or CTM(s). Patient records are updated in accordance with data generated during the current encounter and stored in memory (step 908). Information from one or more sensors may continue to collect information even after the video encounter is completed. This information can be analyzed at a later time by the system and/or an HCP.

The system provides a mechanism for providers to test and diagnosis patients that is applicable to urgent care, physical exams, drug testing and monitoring, remote monitoring, remote elderly care, etc. For example, rather than trying to find an urgent care center, a patient can log into the system and obtain medical care. In life threatening emergencies, however, the patient may be directed to a local hospital that can provide the necessary care.

The system simplifies the prescribing and dispensing of prescription medication that would otherwise require administration by a medical professional. The system also provides a physical presence of one or more of an HCP and/or a HCW at the patient's location. By providing an HCW which can visit a patient, HCPs can spend more time with the patient rather than waste time traveling to and from the patient during a house call. Thus, the system greatly reduces or eliminates the need for a patient to drive to visit a doctor. This saves time, cost, and fuel and provides patients, HCPs, and HCWs with meaningful interaction during an encounter.

An example CTM invite message generated in accordance with the present invention is shown in FIG. 33. The CTM invite message 930 comprises instruction text 932, a listing of one or more CTMs 934 and corresponding selection items, other CTM contact information 938, and patient data access rights selections 939 (e.g., radio buttons, check boxes, etc.).

The listing 934 of CTMs assigned to the patient and/or previously selected is obtained from memory and stored in accordance with patient account information (PAI). The CTMs may have been previously assigned patient data access rights. The selection items (e.g., radio buttons, check boxes, etc.) select one or more of these CTMs. The other CTM contact information 938 includes a text entry area to enter name and contact information (e.g., email, social media, etc.) for another CTM. The patient chooses patient data access rights using selections 939.

After the CTM invite message 930 is generated and/or rendered on the patient's computing device, the patient completes it and transmits it back to the system. The system reads the returned CTM invite message and generates one or more corresponding invite messages which are transmitted to the selected CTMs included. The invite message includes a link for joining the encounter. Note that an invitation prior to the encounter (e.g., when an encounter is a STA) may also be sent, such as at the initiation of an ITA to all CTMs assigned to the patient.

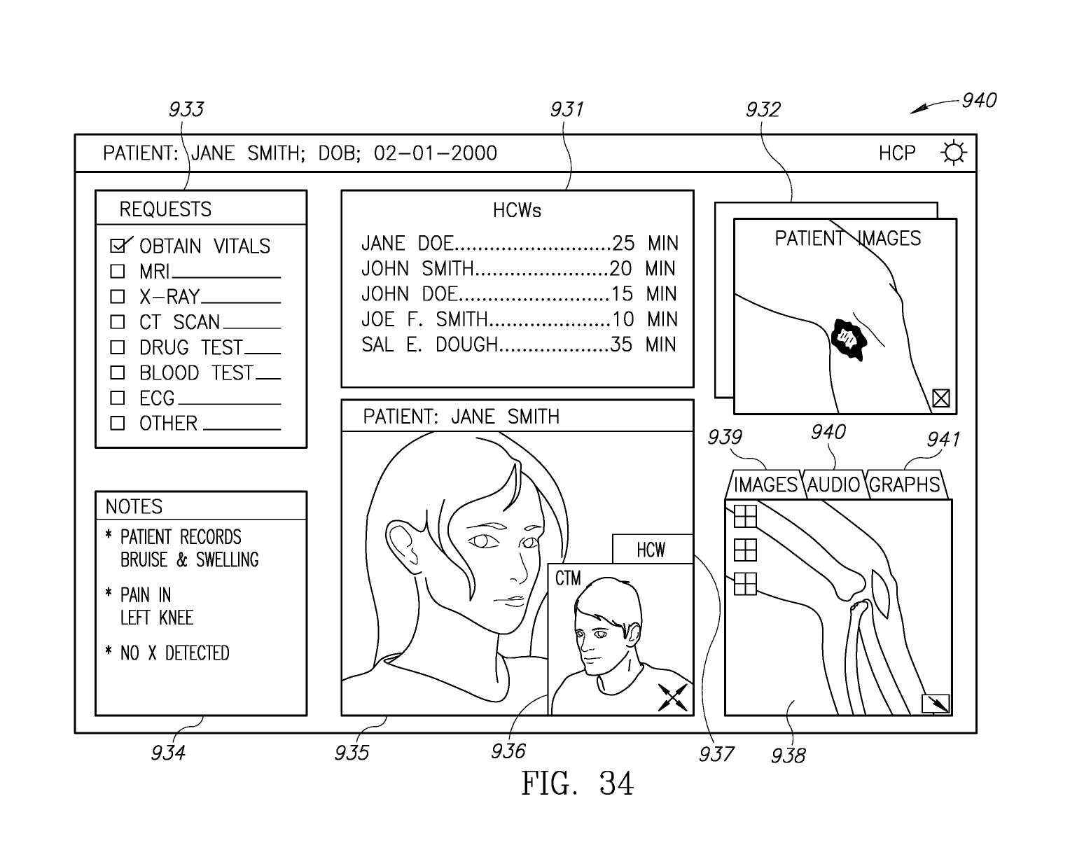

An example GUI rendered on an HCP computing device in accordance with the present invention is shown in FIG. 34. The GUI 940 comprises a plurality of information areas such as windows 933, 931, 932, 934, 935, 936, and 938. The windows may include sub-windows such as window 937 which can be minimized, maximized, moved, resized, and/or closed.

Tabs 939, 940, and 941 are also provided to select between information such as medical images, sound, and graphs, respectively.

Window 933 comprises work request information such as a request for offsite work at the site of the patient and performed by a selected HCW. Examples include obtaining vitals, medical images such as an MRI, X-ray, or CT scan, ultrasound scans, otoscope scan, obtaining audio information (e.g., lungs, heart, etc.) using a stethoscope, blood test, etc. The HCP selects items such as radio buttons, checkboxes, etc. to select work or tasks to be performed. When a work item is selected, the system provides an input area and/or selection area for the HCP to enter specific instructions. For example, the HCP may desire to obtain an MRI of the right knee of the current patient. Accordingly, the HCP may select an MRI selection item and may provide more specific instructions for a HCW to follow once selected. The listing of offsite work may correspond with an offsite work list stored in memory and modifiable by the system and/or user.

Window 931 comprises a listing of available active HCWs and an estimated time of arrival (ETA) to the patient location. The ETA is determined based upon distance to the patient and/or current engagements (e.g., currently obtaining blood sample with 5 minutes allocated to this test, etc.). The HCP then selects an HCW from this list for dispatch to the patient to carry out the desired tests and tasks.

Window 932 comprises one or more available images or video taken by the US of the patient and/or HCW. These images or video are uploaded to the system by the patient prior to or during the encounter, time stamped and stored in accordance with the patient's account. The HCP selects the available images for viewing and/or playback.

Window 934 comprises a text entry area for an HCP to enter notes and/or to select pre-stored note selection items for inclusion into the record of the patient. The pre-stored notes can be listed in any suitable format such as in a list format and may be edited and/or otherwise configured by the HCP and thereafter stored in memory in accordance with the provider information (PI) for the HCP. Thus, each HCP has their own pre-stored notes available for viewing.

Window 935 comprises one or more sub-windows such as windows 936 and 937 each of which includes a real time video of a corresponding party. For example, a live video of the patient is rendered in window 935, a live video of the CTM is rendered in window 936 and another party to the encounter, such as a HCW, is rendered in window 937. Sub-windows 936 and 937 can be superimposed upon the window 935 to conserve display area. The videos are acquired in real time by a US camera of a party to the encounter.

One of the tabs 939, 940, and 941 can be selected to switch between medical images, audio information, and graphs, respectively, obtained by the HCWs. After acquisition, the medical images, audio information, and graphs are processed and/or transmitted to the system for additional processing such as for reconstruction, rendering, and/or storage in memory and linked to PAI of the patient. The information is time stamped, sorted (e.g., into a proper window, etc.), and rendered in real time or from a storage device. Medical image information can be acquired by medical images and rendered in window 938 after acquisition and/or reconstruction.

Audio information can be acquired by an acoustic recording device such as an electronic stethoscope or the like (which generally does not acquire image information). For example, audio information obtained from a stethoscope is stored in an audio file and rendered using a speaker Audio information may be rendered when the audio tab 940 is selected. Play, pause, rewind, fast forward, volume, etc. selection items may be rendered by the system for selection by the HCP.

Graphs can be acquired by a recording device such as an ECG or the like. Data obtained from the recording device is stored in an appropriate file and rendered in any suitable format such as a chart. Graphics information is rendered when tab 941 is selected. Selection items are provided to change chart settings.

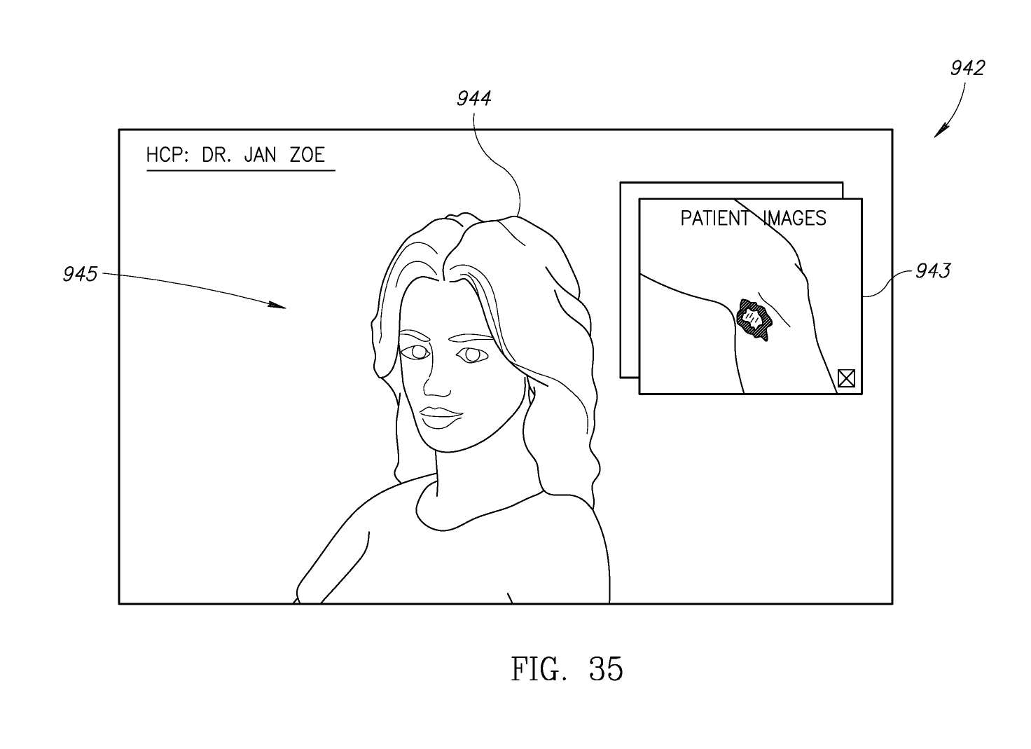

An example GUI rendered on a computing device of the patient in accordance with the present invention is shown in FIG. 35. The GUI 942 comprises a video of the HCP 944 during the encounter. During the above described encounter, the patient can view a live video feed of the HCP conducting the encounter in window 945. The patient can also view information such as images 943 provided by the patient. A menu allows other information to be viewed such as notes, medical images, audio files, graphs (charts), and/or other information generated by the HCP and/or acquired by the HCW. For clarity sake, only images and video of the HCP 944 are shown. The patient, however, can access other related material such as medical images, notes, charts, blood test results, etc.

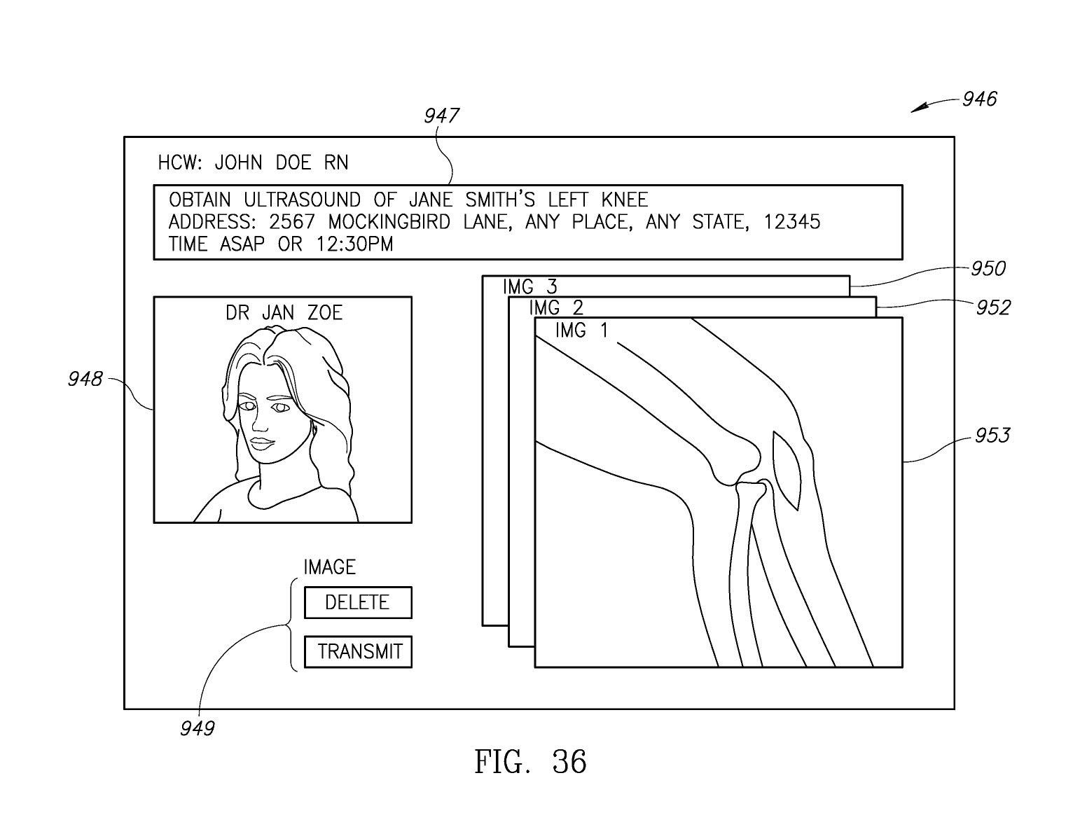

An example GUI rendered on the computing device of the HCW in accordance with the present invention is shown in FIG. 36. The GUI 946 comprises a work order/task window 947 with instructions provided by the HCP, the identity of the patient, and an address of the patient. A link is provided for the patient to select a navigation application (e.g., Google Maps™, Google Maps Navigation™, Waze™, and/or the like) which provides routing information to guide the HCW to the patient location. This link is set by the user choose a preferred guidance application for the HCP. The system provides a live video feed of the HCP in window 948 for the HCW to interact with the user.

One or more images (e.g., ultrasound) acquired by the HCW are displayed in windows 953, 952, and 950. Selection items 949 are provided to delete an image or to transmit the image to the system. Similarly, for signal data (e.g., ECG) the system provides the HCW with the option to delete and/or accept and transmit the data. Once received by the system, the images and/or data samples (e.g., signal data (e.g., ECGs), blood test results, audio information (e.g., stethoscope), etc., are further processed, registered, rendered (e.g., on the UI of the computing device of parties to the encounter such as the HCP) and stored in memory for later use.

The HCP is provided an ability to virtually touch and feel the patient remotely. For example, the HCW may touch and feel the patient using sensors (e.g., glove devices, instruments, etc.) and the data transmitted to the HCP in real time for analysis and diagnosis of medical issues.

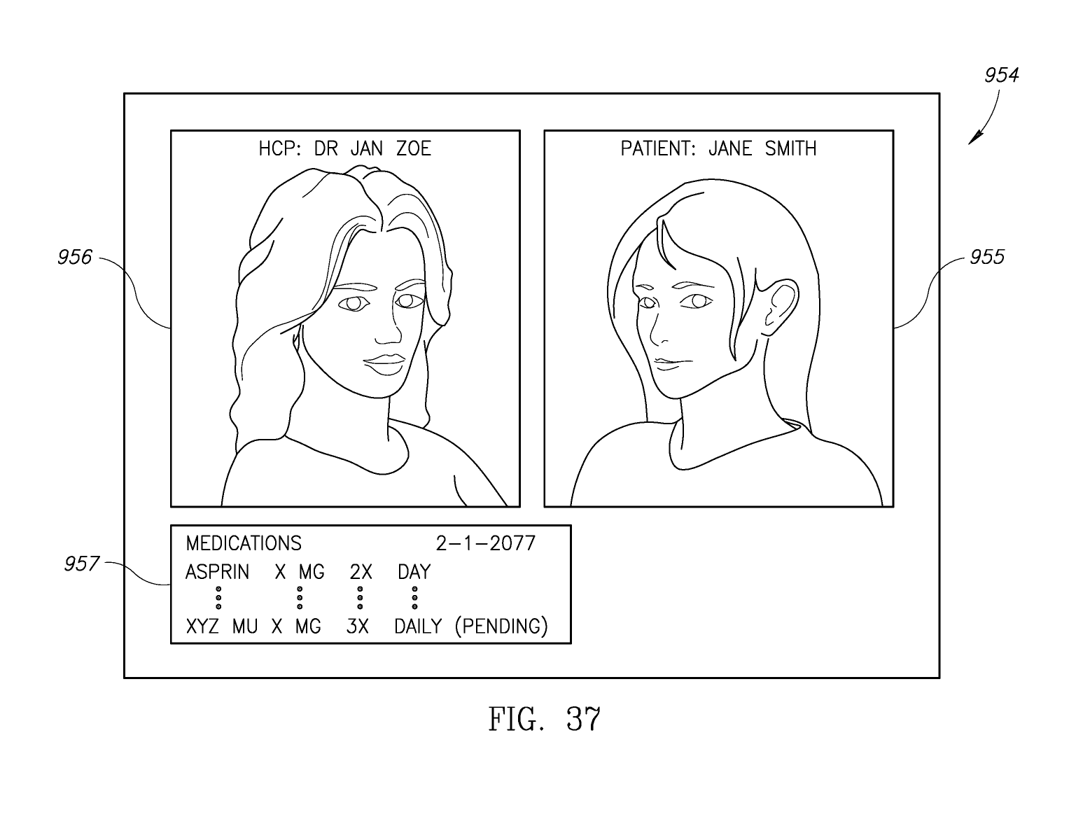

An example GUI rendered on a computing device of the CTM in accordance with the present invention is shown in FIG. 37. The GUI 954 comprises live video of other parties to the encounter such as the HCP in window 956 and the patient in window 955. A medications window 957 displays current medications taken by the patient.

Assuming the current CTM is authorized to view information such as video conference and medications only (as set forth in a CTM access table), the system generates the GUI 954 for the CTM in accordance with their configured authorizations and privileges. Thus, the system limits viewing during the encounter to only video conference and medication information.

Thus, each party to the encounter views information tailored in accordance with their authorizations and privileges. Note that the parties to the encounter may participate using any suitable type of media such as video, audio, texting, etc.

Those skilled in the art will recognize that the boundaries between logic and circuit blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediary components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

In the claims, any reference signs placed between parentheses shall not be construed as limiting the claim. The use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first,” “second,” etc. are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. As numerous modifications and changes will readily occur to those skilled in the art, it is intended that the invention not be limited to the limited number of embodiments described herein. Accordingly, it will be appreciated that all suitable variations, modifications and equivalents may be resorted to, falling within the spirit and scope of the present invention. The embodiments were chosen and described in order to best explain the principles of the invention and the practical application, and to enable others of ordinary skill in the art to understand the invention for various embodiments with various modifications as are suited to the particular use contemplated.

What is claimed is:

1. A method of providing a plurality of virtual immediate waiting rooms, the method comprising:
providing on a server computer, a plurality of software queues, each queue associated with one of a plurality of different legal jurisdictions a patient may be located in;
generating a graphical user interface that when rendered on a computing device displays at least an option for a patient to request an immediate encounter with a healthcare provider;
determining a legal jurisdiction associated with the patent;
placing the patient into a queue corresponding to the legal jurisdiction associated therewith;
granting access to a queue to those healthcare providers licensed in the legal jurisdiction associated therewith;
enabling a healthcare provider to select a patient from one or more queues they have access to and sending a notification thereof to the patient; and
establishing an encounter between the healthcare provider and the selected patient.

2. The method according to claim 1, wherein the plurality of software queues is maintained utilizing one or more databases in communication with the server computer.

3. The method according to claim 1, wherein legal jurisdictions represent different states in the United States of America.

4. The method according to claim 1, wherein said encounter between the selected patient and their associated healthcare provider consists of at least one of a voice call, video call, and text session.

5. The method according to claim 1, further comprising dispatching a healthcare practitioner to a patient location.

6. A method of providing a plurality of virtual scheduled waiting rooms, the method comprising:
providing on a server computer, a plurality of software queues, each queue associated with one of a plurality of healthcare providers;
receiving a request from a patient on a computing device to schedule an encounter with a health care provider;
generating a graphical user interface that when rendered on a computing device displays an option for a patient to select from among a list of healthcare providers that meet one or more criteria;
enabling a patient to select one of the healthcare providers on the list;
sending a request to the server for an encounter between the patient and the selected healthcare provider at a desired date and time;
creating an appointment for the patient with the selected healthcare provider at the desired data and time;
placing the patient in the queue corresponding to the selected healthcare provider a predetermined time prior to the appointment;
sending a notification to the patient when the selected healthcare provider is ready for the encounter; and
establishing an encounter between the selected healthcare provider and the patient.

7. The method according to claim 6, wherein only healthcare providers in the same legal jurisdiction as the patient are provided on the list.

8. The method according to claim 6, wherein the criteria used to create the list consists of at least one of specialty type, name, location, fee, hospital affiliation, medical group membership, insurance accepted, legal jurisdiction and availability on certain dates and times.

9. The method according to claim 6, further comprising sending a notification to the selected healthcare provider when the patient is ready for the encounter.

10. The method according to claim 6, wherein the plurality of software queues is maintained utilizing one or more databases in communication with the server computer.

11. The method according to claim 6, wherein legal jurisdictions represent different states in the United States of America.

12. The method according to claim 6, wherein said encounter between the selected patient and their associated healthcare provider consists of at least one of a voice call, video call, and text session.

13. The method according to claim 6, further comprising dispatching a healthcare practitioner to a patient location.

14. A method of providing a plurality of virtual scheduled waiting rooms for use on a computing device, the method comprising:
generating a graphical user interface that when rendered on the computing device displays an option for a patient to schedule an appointment for an encounter with a healthcare provider;
receiving a request via the graphical user interface to schedule an encounter with a health care provider;
generating a graphical user interface that when rendered on a computing device enables the patient to enter one or more criteria for selecting a healthcare provider;
utilizing the criteria entered by the patient, querying a healthcare provider database located on a server computer accessible over the internet, wherein the server computer comprises a plurality of software queues, each queue associated with one of a plurality of healthcare providers;
receiving in response from the server computer, a list of healthcare providers that satisfy the one or more criteria;
generating a graphical user interface that when rendered on a computing device displays the list of healthcare providers;
generating a graphical user interface that when rendered on a computing device enables the patient to select a healthcare provider on the list;
sending a request to the server computer for an encounter between the patient and the selected healthcare provider at a desired date and time, and in response, the server computer creating an appointment for the patient with the selected healthcare provider at the desired data and time and placing the patient in the queue corresponding to the selected healthcare provider a predetermined time prior to the appointment;
notifying the patient when the selected healthcare provider is ready for the encounter; and
establishing an encounter between the selected healthcare provider and the patient.

15. The method according to claim 14, wherein only healthcare providers in the same legal jurisdiction as the patient are provided on the list.

16. The method according to claim 14, wherein the criteria used to create the list consists of at least one of specialty type, name, location, fee, hospital affiliation, medical group membership, insurance accepted, legal jurisdiction and availability on certain dates and times.

17. The method according to claim 14, further comprising sending a notification to the selected healthcare provider when the patient is ready for the encounter.

18. The method according to claim 14, wherein the plurality of software queues is maintained utilizing one or more databases in communication with the server computer.

19. The method according to claim 14, wherein said encounter between the selected patient and their associated healthcare provider consists of at least one of a voice call, video call, and text session.

20. The method according to claim 14, further comprising dispatching a healthcare practitioner to a patient location.
